SEQUENCE LISTING (5 sequences)

SEQ ID NO: 1 - protein, 214 aa, synthetic construct
  source 1..214: note = Anti-CD3 full length light chain (VL + kappa CL)
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 450 aa, synthetic construct
  source 1..450: note = Anti-CD3 full length heavy chain (VH F2B + silenced IgG4 Fc + knob (S228P, F234A, L235A; T366W)), with C-terminal Lysine (K)
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 472 aa, synthetic construct
  source 1..472: note = BCMA bivalent heavy chain (ABBV-383 with GS1) + silenced IgG4 Fc hinge CH2 CH3, hole (S228P, F234A, L235A, T366S, L368A, Y407V, with C-terminal Lysine (K)
EVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSGGGGSEVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 4 - protein, 449 aa, synthetic construct
  source 1..449: note = Anti-CD3 full length heavy chain (VH F2B + silenced IgG4 Fc + knob (S228P, F234A, L235A; T366W)), without C-terminal Lysine (K)
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 5 - protein, 471 aa, synthetic construct
  source 1..471: note = BCMA bivalent heavy chain (TNB-383B w GS1) + silenced IgG4 Fc hinge CH2 CH3, hole (S228P, F234A, L235A, T366S, L368A, Y407V, without C-terminal Lysine (K)
EVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSGGGGSEVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

(19) United States
(12) Patent Application Publication
(10) Pub. No.: US 2025/0177520 A1
(43) Pub. Date: Jun. 5, 2025

(54) METHODS FOR THE TREATMENT OF MULTIPLE MYELOMA

(71) Applicant: TENEOONE, INC., North Chicago, IL (US)

(72) Inventors: Chetasi TALATI, Collierville, TN (US); Rajvineeth POTHACAMURY, Lake Bluff, IL (US); Jeremy ROSS, Mundelein, IL (US); Akshanth R. POLEPALLY, Redwood City, CA (US); Anders SVENSSON, Wilmette, IL (US)

(73) Assignee: TENEOONE, INC., North Chicago, IL (US)

(21) Appl. No.: 18/963,298

(22) Filed: Nov. 27, 2024

Related U.S. Application Data
(60) Provisional application No. 63/678,022, filed on Jul. 31, 2024.
(60) Provisional application No. 63/649,873, filed on May 20, 2024.
(60) Provisional application No. 63/564,353, filed on Mar. 12, 2024.
(60) Provisional application No. 63/605,368, filed on Dec. 1, 2023.

(51) Int. Cl. A61K 39/395; A61K 31/573; A61K 39/00; C07K 16/28

(52) U.S. Cl. CPC A61K 39/3955; A61K 31/573; C07K 16/2809; C07K 16/2878; A61K 2039/545; C07K 2317/31; C07K 2317/52; C07K 2317/524; C07K 2317/526; C07K 2317/53; C07K 2317/90

(57) ABSTRACT

Methods of treating multiple myeloma by administering a bispecific antibody that binds to CD3 and BCMA to a patient in need are provided.

### CROSS-REFERENCE TO RELATED APPLICATIONS

This application claims the benefit of U.S. Ser. No. 63/605,368 filed Dec. 1, 2023, U.S. Ser. No. 63/564,353 filed Mar. 12, 2024, U.S. Ser. No. 63/649,873 filed May 20, 2024, and U.S. Ser. No. 63/678,022 filed Jul. 31, 2024, the disclosure of each of which is incorporated by reference herein in its entirety.

REFERENCE TO SEQUENCE LISTING SUBMITTED ELECTRONICALLY

This application contains an electronic Sequence Listing which has been submitted in XML file format via Patent Center, the entire content of which is incorporated by reference herein in its entirety. The Sequence Listing XML file submitted via Patent Center is entitled “13371-316-999_SEQLISTING.xml”, was created on Oct. 30, 2024, and is 8,631 bytes in size.

### 1. FIELD

The present application pertains to, among other things, improved methods of treating multiple myeloma (MM) by administering a bispecific antibody to a patient in need. In specific embodiments, the bispecific antibody binds to CD3 and BCMA (a CD3×BCMA bispecific antibody).

### 2. BACKGROUND

MM is a plasma cell malignancy and the second most frequent hematopoietic cancer with an annual incidence of ˜30,000 in the United States (US). The disease is characterized by the proliferation of clonal plasma cells in the bone marrow and frequently accompanied by the production of a monoclonal immunoglobulin. More than 80% of patients are >60 years of age, with a median age of onset of 68 years; approximately 2% of cases are diagnosed prior to the age of 40 (Jemal A et al., Cancer Statistics, 2008; 58 (2): 71-96.; Waxman A J et al., Blood. 2010; 116 (25): 5501-6; Pulte D et al., The Oncologist. 2011; 16 (11): 1600-3.). The disease primarily localizes to the bones and bone marrow, with resultant cytopenias, bone pain, fractures, infections, hypercalcemia, and renal failure. In addition, serious neurological sequelae can result from pathological fractures in the vertebral bodies.

Use of bispecific antibodies, however, to treat MM poses the risks of dose-limiting toxicities such as cytokine release syndrome (CRS), immune effector cell-associated neurotoxicity syndrome (ICANS), or both. In fact, per the label on certain CD3×BCMA bispecific antibodies (e.g., teclistamab and elranatamab), patient accessibility may be limited because of the boxed warnings for CRS and ICANS. Furthermore, the dosing regimens for teclistamab and elranatamab require that two step-up doses be given in the week prior to the first maintenance dose, with weekly to biweekly (for elranatamab, after 24 weeks based on response) dosing thereafter. Patients receiving teclistamab are required to remain within proximity of a healthcare facility (in the EU) or hospitalized (in the US) for 48 hours after administration of all doses within the first weekly dosing regimen (two step-up doses and the first maintenance dose). Patients receiving elranatamab are required to be hospitalized for 48 hours and 24 hours after the second step-up dose in the first cycle for monitoring-particularly for CRS. Similarly, patients receiving the CD3×BCMA bispecific antibody linvoseltamab require hospitalization for 24 hours after each step-up dose for similar monitoring. Each of teclistamab, elranatamab and linvoseltamab require two step-up doses.

Thus, given the shortcomings of the currently approved therapies, there remains an unmet need for effective therapies for advanced MM, including new therapies with an improved CRS profile and dosing regimens that provide durable efficacy, favorable safety (for example, that reduces CRS), or simplified dosing regimens The present disclosure addresses these unmet needs, among others.

### 3. SUMMARY

In one aspect, provided herein is A method for safely and effectively treating a human patient with multiple myeloma (MM) comprising administering to the patient during a first cycle comprising: i) a first premedication dose of a corticosteroid, followed by a step-up dose of about 2 mg of etentamig, and ii) a modified premedication dose regimen of a corticosteroid, followed by a maintenance dose of about 60 mg of etentamig, wherein the first cycle has a duration of about four weeks, and wherein said method reduces the incidence of one or more adverse events as compared to the administration of the maintenance dose without administration of a step-up dose.

In embodiments, the corticosteroid is dexamethasone. In embodiments, the first premedication dose of a corticosteroid comprises about 10 mg of dexamethasone. In embodiments, the about 10 mg of dexamethasone is orally administered to the patient about 15 to 60 minutes prior to administering the step-up dose of the etentamig.

In embodiments, the corticosteroid is selected from the group consisting of: dexamethasone, hydrocortisone, prednisolone, and methylprednisolone.

In embodiments, the first premedication dose of a corticosteroid comprises one of about 10 mg of dexamethasone, about 250 mg of hydrocortisone, about 62 mg of prednisolone, or about 50 mg of methylprednisolone. In embodiments, the about 10 mg of dexamethasone, about 250 mg of hydrocortisone, about 62 mg of prednisolone, or about 50 mg of methylprednisolone, is orally administered to the patient about 15 to 60 minutes prior to administering the step-up dose of the etentamig.

In embodiments, the modified premedication dose regimen of a corticosteroid comprises about 36 mg of dexamethasone. In embodiments, the about 36 mg of dexamethasone comprises three separate administrations of dexamethasone. In embodiments, one administration of the dexamethasone comprises oral administration of about 8 mg of dexamethasone about 12-16 hours prior to administration of the maintenance dose of the etentamig.

In embodiments, one administration of the dexamethasone comprises oral administration of about 8 mg of dexamethasone about 2-5 hours prior to administration of the maintenance dose of the etentamig. In embodiments, one administration of the dexamethasone comprises oral or intravenous administration of about 20 mg of dexamethasone about 15-60 minutes prior to administration of the maintenance dose of the etentamig.

In embodiments, the modified premedication dose regimen of a corticosteroid comprises one of about 36 mg of dexamethasone, about 900 mg of hydrocortisone, about 225 mg of prednisolone, or about 180 mg of methylprednisolone. In embodiments, the about 36 mg of dexamethasone, the about 900 mg of hydrocortisone, the about 225 mg of prednisolone, or the about 180 mg of methylprednisolone, comprises three separate administrations. In embodiments, one administration of the dexamethasone, hydrocortisone, prednisolone, or methylprednisolone comprises oral administration of one of about 8 mg of dexamethasone, about 200 mg of hydrocortisone, about 50 mg of prednisolone, or about 40 mg of methylprednisolone, about 12-16 hours prior to administration of the maintenance dose of the etentamig. In embodiments, one administration of the dexamethasone, hydrocortisone, prednisolone, or methylprednisolone comprises oral administration of one of about 8 mg of dexamethasone, about 200 mg of hydrocortisone, about 50 mg of prednisolone, or about 40 mg of methylprednisolone, about 2-5 hours prior to administration of the maintenance dose of the etentamig. In embodiments, one administration of the dexamethasone, hydrocortisone, prednisolone, or methylprednisolone comprises oral or intravenous administration of one of about 20 mg of dexamethasone, about 500 mg of hydrocortisone, about 125 mg of prednisolone, or about 100 mg of methylprednisolone, about 15-60 minutes prior to administration of the maintenance dose of the etentamig.

In embodiments, the step-up dose of the etentamig is administered on day 1 of the first cycle. In embodiments, the maintenance dose of the etentamig is administered on day 4 of the first cycle. In embodiments, the maintenance dose of the etentamig is administered on a single day of any of days 4-14 of the first cycle.

In embodiments, the method further comprising one or more subsequent cycles after the first cycle. In embodiments, the maintenance dose of the etentamig is administered on day 1 of each subsequent cycle after the first cycle.

In embodiments, the adverse event is CRS. In embodiments, the adverse event comprises Grade 1, Grade 2, or Grade 3 CRS. In embodiments, the adverse event comprises at least one of CRS, neutropenia, anemia, thrombocytopenia, fatigue, diarrhea, decreased neutrophil count, nausea, and decreased lymphocyte count.

In embodiments, the MM is relapsed or refractory MM. In embodiments, the patient has received at least one prior line of therapy, at least two prior lines of therapy, or at least three prior lines of therapy.

In embodiments, the patient is an individual within a population of subjects receiving said treatment, and wherein the proportion of subjects within said population that achieves an objective response rate (ORR) is at least about 60%. In embodiments, the patient is an individual within a population of subjects receiving said treatment, and wherein the proportion of subjects within said population that achieves a very good partial response (VGPR) is at least about 50%. In embodiments, the patient is an individual within a population of subjects receiving said treatment, and wherein the proportion of subjects within said population that achieves a complete response (CR) is at least about 5%.

In another aspect, provided herein is a method of therapeutically effectively treating MM in a human patient in need thereof, wherein the method comprises: (A) during a first cycle, administering to the patient a step-up dose of etentamig at a dose of about 2 mg, and administering to the patient a maintenance dose of the etentamig at a dose of about 60 mg; and (B) during a subsequent cycle, administering to the patient the maintenance dose of the etentamig, wherein, each cycle comprises about four weeks, and wherein the patient achieves an objective response, thereby treating said MM.

In embodiments, the method further comprises one or more subsequent cycles, wherein each subsequent cycle has a duration of about four weeks, and wherein each subsequent cycle comprises the maintenance dose of the etentamig. In embodiments, the patient is an individual within a population of subjects receiving said treatment, and wherein the proportion of subjects within said population that achieves an ORR is at least about 60%. In embodiments, the patient is an individual within a population of subjects receiving said treatment, and wherein the proportion of subjects within said population that achieves a VGPR is at least about 50%. In embodiments, the patient is an individual within a population of subjects receiving said treatment, and wherein the proportion of subjects within said population that achieves a CR is at least about 5%.

In another aspect, provided herein is a method of therapeutically effectively treating MM in a human patient in need thereof, wherein the method comprises: during a first cycle, administering to the patient about 62 mg of etentamig; and during one or more subsequent cycles, administering to the patient about 60 mg of the etentamig, wherein each cycle has a duration of about four weeks, thereby treating said MM.

In embodiments, the method further comprises, during the first cycle, administering to the patient about 46 mg of dexamethasone. In embodiments, the patient is an individual within a population of subjects receiving said treatment, and wherein the proportion of subjects within said population that achieves an ORR is at least about 60%. In embodiments, the patient is an individual within a population of subjects receiving said treatment, and wherein the proportion of subjects within said population that achieves a VGPR is at least about 50%. In embodiments, the patient is an individual within a population of subjects receiving said treatment, and wherein the proportion of subjects within said population that achieves a CR is at least about 5%.

A method for the safe and effective treatment of MM in a patient in need thereof, wherein the method comprises: (A) during a first cycle, i) administering to the patient a first premedication dose of a corticosteroid, ii) subsequently administering to the patient a step-up dose of about 2 mg of etentamig, iii) subsequently administering to the patient one or more additional premedication doses of a corticosteroid, iv) subsequently administering to the patient a maintenance dose of about 60 mg of the etentamig; and (B) during one or more subsequent cycles, administering to the patient the maintenance dose of the etentamig; wherein each of the first and subsequent cycles have a duration of approximately four weeks; and, wherein the method results in a reduced incidence of one or more adverse events as compared to a second method comprising administering etentamig without one or more doses of a corticosteroid.

In embodiments, the corticosteroid is dexamethasone. In embodiments, the first premedication dose comprises 10 mg of dexamethasone administered orally. In embodiments, the one or more additional premedication doses comprises two oral doses of 8 mg dexamethasone, and one oral or intravenous dose of 20 mg dexamethasone. In embodiments, the first 8 mg dexamethasone dose is administered orally about 12-16 hours prior to the administration of the etentamig maintenance dose; the second 8 mg dexamethasone dose is administered orally about 2-5 hours prior to administration of the etentamig maintenance dose; and, the 20 mg dexamethasone dose is administered orally or intravenously less than one hour prior to the administration of the etentamig maintenance dose. In embodiments, the patient is administered a total of about 20 to about 60 mg dexamethasone during the first cycle.

In embodiments, the one or more adverse events are selected from the group consisting of CRS, neutropenia, anemia, thrombocytopenia, fatigue, diarrhea, decreased neutrophil count, nausea, and decreased lymphocyte count. In embodiments, the adverse event is CRS.

In embodiments, the patient is an individual within a population of subjects receiving said treatment, and wherein the proportion of subjects within said population that achieves an ORR is at least about 60%. In embodiments, the patient is an individual within a population of subjects receiving said treatment, and wherein the proportion of subjects within said population that achieves a VGPR is at least about 50%. In embodiments, the patient is an individual within a population of subjects receiving said treatment, and wherein the proportion of subjects within said population that achieves a CR is at least about 5%.

In another aspect, provided herein is a method for the safe and effective treatment of MM in a patient in need thereof, wherein the method comprises: (A) during a first cycle, i) administering to the patient one or more premedication doses of a corticosteroid, ii) administering to the patient a step-up dose of etentamig at a dose of about 2 mg, iii) after the step-up dose of etentamig, administering to the patient a maintenance dose of the etentamig at a dose of about 60 mg; and (B) during one or more subsequent cycles, administering to the patient the maintenance dose of the etentamig; wherein the first cycle has a duration of approximately four weeks; and, wherein, when the method is used to treat MM in a population of patients, the method results in a reduction of one or more adverse events as compared to a second method comprising administering etentamig without one or more doses of a corticosteroid.

In embodiments, the method results in a statistically significant reduction in one or more adverse events as compared to a second method comprising administering etentamig without one or more doses of a corticosteroid. In embodiments, the corticosteroid is dexamethasone.

In embodiments, the one or more premedication doses comprises one oral dose of 10 mg dexamethasone, two oral doses of 8 mg dexamethasone, and one oral or intravenous dose of 20 mg dexamethasone. In embodiments, the 10 mg dexamethasone dose is administered about one hour or less prior to administration of the step-up dose of the etentamig; the first 8 mg dexamethasone dose is administered orally about 12-16 hours prior to the administration of the maintenance dose of the etentamig; the second 8 mg dexamethasone dose is administered orally about 2-5 hours prior to administration of the maintenance dose of the etentamig; and, the 20 mg dexamethasone dose is administered orally or intravenously less than one hour prior to the administration of the maintenance dose of the etentamig.

In embodiments, the one or more adverse events are selected from the group consisting of CRS, neutropenia, anemia, thrombocytopenia, fatigue, diarrhea, decreased neutrophil count, nausea, and decreased lymphocyte count. In embodiments, the adverse event is CRS. In embodiments, the total incidence of CRS is approximately 30%. In embodiments, the incidence of grade 3 CRS is less than 5%. In embodiments, the incidence of grade 2 CRS is less than 10%. In embodiments, the incidence of grade 1 CRS is less than 30%.

In embodiments, the patient is an individual within the population of patients receiving said treatment, and wherein the proportion of subjects within said population that achieves an ORR is at least about 60%. In embodiments, the patient is an individual within the population of patients receiving said treatment, and wherein the proportion of subjects within said population that achieves a VGPR is at least about 50%. In embodiments, the patient is an individual within the population of patients receiving said treatment, and wherein the proportion of subjects within said population that achieves a CR is at least about 5%.

In embodiments, the method further comprises one or more subsequent cycles wherein the patient is administered the maintenance dose of the etentamig.

In another aspect, provided herein is a method for the safe and effective treatment of MM in a patient in need thereof, comprising administering to the patient (A) during a first cycle: on day 1 (but prior to step ii)), an oral premedication dose of about 10 mg of dexamethasone, on day 1, a step-up dose of about 2 mg of etentamig, on day 3 (or up to day 13 but prior to step v)), an oral premedication dose of about 8 mg of dexamethasone, on day 3 (or up to day 13 but prior to step v)), an oral premedication dose of about 8 mg of dexamethasone, on day 4 (or up to day 14), an oral or intravenous premedication dose of about 20 mg of dexamethasone, on day 4 (or up to day 14), a maintenance dose of about 60 mg of the etentamig; and (B) during a subsequent cycle: on day 1, the maintenance dose of the etentamig, wherein each cycle comprises about 28 days, and wherein the method results in a greater reduction in CRS without reduction of overall response rate (ORR) in treating said MM as compared to administration of the maintenance dose of the etentamig without the step-up dose of the etentamig during the first cycle.

These and further aspects will be further explained in the rest of the disclosure, including the Examples.

### 4. BRIEF DESCRIPTION OF THE DRAWINGS

FIGS. 1A-1C are graphs showing response rates in subjects receiving 20 mg Q3W, 40 mg Q3W, 60 mg Q3W or 60 mg Q4W. Stringent complete response/complete response (sCR/CR), VGPR, and partial response (PR) response rates (%) are shown according to overall follow-up (FIG. 1A) or after 3 cycles (FIG. 1B). Median PFS (95 CI) in patients receiving Q3W doses was 3.8 months (2.4, 7.9) for 20 mg, 13.7 (5.0, —) for 40 mg, 11.2 (4.8,--) for 60 mg, and not reached (3.7,--) in patients treated with 60 mg Q4W (FIG. 1C).

FIG. 2 is a graph showing the incidence and severity of CRS following administration of etentamig according to the indicated dosing regimen.

FIGS. 3A-3C are graphs showing the incidence of CRS (FIG. 3A), the incidence of grade 3/4 cytopenia (neutropenia, anemia, thrombocytopenia) (FIG. 3B), and the incidence and severity of infections (FIG. 3C) following administration of etentamig according to the indicated dosing regimen.

FIGS. 4A-4C are graphs showing exposure-efficacy for ORR (FIG. 4A), very good partial response rate (>VGPR) (FIG. 4B), and stringent complete response/complete response (sCR/CR) (FIG. 4C) analyses using free etentamig Cavg as exposure metric.

FIGS. 5A-5D are graphs showing exposure-safety analysis for Grade ≥3 Neutropenia (FIG. 5A and FIG. 5C) and any-grade CRS (FIG. 5B and FIG. 5D) using observed total cycle 1 etentamig Cmax as exposure metric.

FIGS. 6A-6B are graphs showing that higher baseline sBCMA appeared to reduce free PK exposures (FIG. 6B) only and not impact total PK exposures (FIG. 6A).

FIG. 7 is a graph showing that median sBCMA levels at baseline were highly variable among patients, but did not associate with clinical response (>partial response [PR]) to etentamig at 60 mg Q4W.

FIG. 8 is a graph showing that reduction in sBCMA levels over time was associated with clinical response.

FIGS. 9A-9D are graphs showing that treatment with etentamig led to a rapid and transient increase of IL-6 (FIG. 9A) and TNF-α (FIG. 9B) proinflammatory cytokines, and the IL-6 (FIG. 9C) and TNF-α (FIG. 9D) cytokine peak levels correlated with CRS occurrence and clinical response across dose levels.

FIGS. 10A-10B are graphs showing that treatment with etentamig at 60 mg Q4W promoted T-cell redistribution and expansion for both CD4 T-cells (FIG. 10A) and CD8 T-cells (FIG. 10B).

FIGS. 11A-11D are graphs showing the frequency of baseline CD8+ T-cell exhaustion (PD-1/TIM3) did not impact clinical response at 60 mg Q4W etentamig.

FIG. 12 is a heatmap showing that premedication with a 36 mg dexamethasone regimen reduced the expression of myeloid-derived inflammatory markers (rightmost vertical rectangle indicates premedication with a 36 mg dexamethasone regimen; lower right square indicates myeloid derived inflammatory markers).

FIGS. 13A-13B are graphs showing a comparison of dosing regimens and observed incidence of CRS (FIG. 13A) and incidence of ICANS (FIG. 13B).

FIGS. 14A-14C are graphs showing percentage of patients demonstrating elevated biomarkers as a function of dose cohort for percent of patients with grade 3/4 elevations in aspartate to alanine amino-transferase levels (AST/ALT)) (FIG. 14A); grade 3/4 thrombocytopenia (FIG. 14B); and grade 2+creatinine (FIG. 14C).

FIG. 15 is a chart showing peak levels of pro-inflammatory cytokines across dose cohorts.

FIGS. 16A-16H are graphs showing maximal concentrations (Cmax) levels of pro-inflammatory cytokines and interleukins (IL) across timepoints by dosage cohort for IL-8 (FIG. 16A and FIG. 16B, respectively), IL-10 (FIG. 16C and FIG. 16D, respectively), TNF-α (FIG. 16E and FIG. 16F, respectively), and IL-6 (FIG. 16G and FIG. 16H, respectively).

FIGS. 17A-17D are graphs demonstrating the biphasic release of pro-inflammatory cytokines associated with SUD of etentamig for IL-6 IL-8, IL-10, and TNF-α (FIG. 17A, FIG. 17B, FIG. 17C and FIG. 17D, respectively).

FIGS. 18A-18C are graphs showing pharmacokinetic profiles of exposure response (ER) curves of any grade CRS to demonstrate the probability of occurrence of any grade CRS in subjects receiving SUD or modified premedication dosing regimen with DEX and a combination of both SUD and 10 mg DEX premedication/modified premedication dosing regimen with DEX.

### 5. DETAILED DESCRIPTION

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included herein.

All references cited throughout the disclosure, including patent applications and publications, are incorporated by reference herein in their entirety.

### 5.1 Terminology

The term “about” as used herein is intended to mean within 20%, within 15%, within 10%, within 9%, within 8%, within 7%, within 6%, within 5%, within 4%, within 3%, within 2%, within 1%, or less of a given value or range.

Antibody residues herein are numbered according to the Kabat numbering system and the EU numbering system. The Kabat numbering system is generally used when referring to a residue in the variable domain (approximately residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies mean residue numbering by the Kabat numbering system. Unless stated otherwise herein, references to residue numbers in the constant domain of antibodies mean residue numbering by the EU numbering system.

The term “CD3” refers to the human CD3 protein multi-subunit complex. The CD3 protein multi-subunit complex is composed of 6 distinctive polypeptide chains. These include a CD3γ chain (SwissProt P09693), a CD38 chain (SwissProtP04234), two CD3ε chains (SwissProt P07766), and one CD3ζ chain homodimer (SwissProt 20963), and a T-cell receptor (α/β or γ/δ) heterodimer. The term “CD3” includes any CD3 variant, isoform and species homolog that is naturally expressed by cells (including T-cells) or can be expressed on cells transfected with genes or cDNA encoding those polypeptides, unless noted. In specific embodiments, the CD3 is human CD388.

A “CD3×BCMA bispecific antibody” is a bispecific antibody, which comprises two different antigen-binding regions, one of which binds to the antigen human CD3 and one of which binds to human BCMA.

The term “BCMA” as used herein relates to human B-cell maturation antigen, also known as BCMA, CD269, and TNFRSF17 (UniProt Q02223), which is a member of the tumor necrosis receptor superfamily that is preferentially expressed in differentiated plasma cells. The extracellular domain of human BCMA consists, according to UniProt of amino acids 1-54 (or 5-51).

A “multi-valent” antibody has two or more binding sites. Thus, the terms “bivalent,” “trivalent,” and “tetravalent” refer to the presence of two binding sites, three binding sites, and four binding sites, respectively. Thus, a bispecific antibody according to the disclosure is at least bivalent and may be trivalent, tetravalent, or otherwise multi-valent. A bivalent antibody in accordance with embodiments of the disclosure may have two binding sites to the same epitope (i.e., bivalent, monoparatopic), or to two different epitopes (i.e., bivalent, biparatopic).

The term “effector cell” refers to an immune cell which is involved in the effector phase of an immune response, as opposed to the cognitive and activation phases of an immune response. Some effector cells express specific Fc receptors and carry out specific immune functions. In embodiments, an effector cell such as a natural killer cell is capable of inducing antibody-dependent cellular cytotoxicity (ADCC). For example, monocytes and macrophages, which express FcR, are involved in specific killing of target cells and presenting antigens to other components of the immune system or binding to cells that present antigens. In embodiments, an effector cell may phagocytose a target antigen or target cell.

The term “human effector cells” refers to leukocytes that express receptors such as T-cell receptors or FcRs and perform effector functions. Preferably, the cells express at least FcγRIII and perform ADCC effector function. Examples of human leukocytes which mediate ADCC include natural killer (NK) cells, monocytes, cytotoxic T-cells and neutrophils. The effector cells may be isolated from a native source thereof, e.g., from blood or PBMCs as described herein.

The term “immune cell” is used herein in the broadest sense, including, without limitation, cells of myeloid or lymphoid origin, for instance lymphocytes (such as B-cells and T-cells including cytolytic T-cells (CTLs)), killer cells, natural killer (NK) cells, macrophages, monocytes, eosinophils, polymorphonuclear cells, such as neutrophils, granulocytes, mast cells, and basophils.

Antibody “effector functions” refer to those biological activities attributable to the Fc region (a native sequence Fc region or amino acid sequence variant Fc region) of an antibody. Examples of antibody effector functions include Clq binding; complement dependent cytotoxicity (CDC); Fc receptor binding; antibody-dependent cell-mediated cytotoxicity (ADCC); phagocytosis; down regulation of cell surface receptors (e.g., B-cell receptor; BCR), etc.

“Antibody-dependent cell-mediated cytotoxicity” and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Ravetch and Kinet, Annu. Rev. Immunol 9:457-92 (1991) (at p. 464 (Table 3)). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

“Complement dependent cytotoxicity” or “CDC” refers to the ability of a molecule to lyse a target in the presence of complement. The complement activation pathway is initiated by the binding of the first component of the complement system (Clq) to a molecule (e.g., an antibody) complexed with a cognate antigen. To assess complement activation, a CDC assay, e.g., as described in Gazzano-Santoro et al., J. Immunol. Methods 202:163 (1996), may be performed.

The term “maintenance dose” refers to a dose of a therapeutic agent (e.g., a CD3×BCMA bispecific antibody) that is given to a subject to treat a disease (e.g., MM) and may be administered in subsequent cycles following a first cycle, and in a first cycle (e.g., together with a step-up dose) as the therapeutically effective amount.

The term “monotherapy” refers to the use of a single agent (e.g., a CD3×BCMA bispecific antibody provided herein (e.g., etentamig)), without a second active agent, to treat the same indication, e.g., the same MM. In embodiments, the term “monotherapy” does not exclude one or more additional agent(s) from being administered to a subject if the one or more additional agent(s) is/are not administered for treating the MM in the subject. In embodiments, the term “monotherapy” does not exclude premedication (e.g., premedication with antihistamine, acetaminophen, hypertension agents, steroids, and the like). For example, in embodiments, the term “monotherapy” does not exclude one or more additional agent(s) used to prevent or ameliorate injection site side effects, fever, and/or any other side effect associated with administering a CD3×BCMA bispecific antibody to a subject. In embodiments, the term “monotherapy” does not exclude treatment with one or more additional agent(s) used for treating or ameliorating another disease or disorder in the subject (e.g., a disease or disorder that is not the MM that a CD3×BCMA bispecific antibody is being used to treat). In embodiments, the term “monotherapy” does not exclude previous treatment with another agent that was used for treating or attempting to treat the MM in a subject (e.g., a subject undergoing monotherapy treatment with a CD3×BCMA bispecific antibody can be a subject that was previously treated with another agent for the MM; e.g., the CD3×BCMA bispecific antibody treatment is a second, third, or fourth line of therapy). In embodiments, a subject receiving monotherapy treatment with the CD3×BCMA bispecific antibody is a subject with MM that is resistant to agents that were previously used for treating the MM.

The term “premedication” refers to one or more additional agent(s) administered to a subject for a purpose that is not primarily or solely treating the MM in the subject. For example, premedication may refer to one or more additional agent(s) used to prevent or ameliorate injection site side effects, fever, and/or any other side effect associated with a CD3×BCMA bispecific antibody. Premedication may also refer to one or more additional agent(s) used for primarily treating or ameliorating a disease or disorder in the subject that is not the MM. In embodiments, a premedication is administered using a premedication regimen.

The term “step-up dose,” which may also be referred to as a “priming” dose, refers to an initial dose or doses of a therapeutic agent (e.g., a CD3×BCMA bispecific antibody) given in a first cycle to prime the subject before administering a maintenance dose. In the first cycle, together the step-up dose(s) and maintenance dose may be a therapeutically effective amount.

The terms “treatment,” “treating,” and the like are used herein to generally mean obtaining a desired pharmacologic and/or physiologic effect. “Treatment” as used herein covers any treatment of a disease in a human and includes inhibiting the disease, i.e., arresting its development; or relieving the disease, i.e., causing regression of the disease. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease.

A “therapeutically effective amount” is an amount of active agent that imparts therapeutic benefit to a subject as part of a course of treatment. Therapeutic benefit may be a safety or efficacy benefit. For example, a “therapeutically effective amount” is an amount that induces, ameliorates or otherwise causes an improvement in the pathological symptoms, disease progression, or physiological conditions associated with a disease. In embodiments, the term “therapeutically effective amount” is an amount of active agent that obtains a desired pharmacological and/or physiological effect.

The term “characterized by expression of BCMA” broadly refers to any disease or disorder in which BCMA expression is associated with or involved with one or more pathological processes that are characteristic of the disease or disorder. Such disorders include, but are not limited to, B-cell neoplasms, including MM.

The terms “subject,” “individual,” and “patient” are used interchangeably herein to refer to a human being assessed for treatment and/or being treated.

Abbreviations used herein include the following: B (Terminal phase elimination rate constant); ADA (Antidrug antibody); ADCC (Antibody-dependent cell cytotoxicity); AE (Adverse event); ANC (Absolute neutrophil count); APRIL (A proliferation inducing ligand); AST (Aspartate aminotransferase); AUC (Area under the concentration-time curve); AUCt (Area under the serum concentration-time curve from time zero to time of last measurable concentration); BCMA (B-cell maturation antigen (also called TNFRSF17)); BUN (Blood urea nitrogen); CAR (Chimeric antigen receptor); CBR (Clinical benefit rate); CI (Confidence interval); CL (Clearance); Cmax (Maximum observed serum concentration); CNS (Central nervous system); CR (Complete response); CRS (Cytokine release syndrome); Css (Steady state concentration); CT (Computed tomography); CTCAE (Common Terminology Criteria for Adverse Events); DEX (dexamethasone); DLT (Dose limiting toxicity); DNA (Deoxyribonucleic acid); DOR (Duration of Response); ECG (Electrocardiogram); ECHO (Echocardiogram); ECOG (Eastern Cooperative Oncology Group); eCRF (Electronic Case Report Form); EDC (Electronic Data Capture); EE (Efficacy Evaluable); ELISA (Enzyme-linked immunosorbent assay); EOT (End of treatment); FFPE (Formalin-fixed paraffin embedded); FIH (First-in-human); FISH (Fluorescence in situ hybridization); FLC (Free light chain); GCP (Good Clinical Practice); HAV-IgM (Hepatitis A virus immunoglobulin M); HBsAg (Hepatitis B surface antigen); HBV (Hepatitis B virus); HCV (Hepatitis C virus); HCV Ab (Hepatitis C virus antibody); HIV (Human immunodeficiency virus); IB (Investigator's Brochure); ICH (International Conference on Harmonization); IEC (Independent Ethics Committee); IMID (Immunomodulatory imide); IMWG (International Myeloma Working Group); INR (International normalized ratio); IRB (Institutional Review Board); IV (Intravenous); LDH (Lactate dehydrogenase); mAb (Monoclonal antibody); MABEL (Minimal anticipated biological effect level); MedDRA (Medical Dictionary for Regulatory Activities); MM (Multiple myeloma); MPDR (modified premedication dose regimen); MR (Minor response); MRI (Magnetic Resonance Imaging); MTD (Maximum tolerated dose); MUGA (Multiple gated acquisition scan); NCI (National Cancer Institute); MTD (Maximum Tolerated Dose); NCA (Noncompartmental analysis); ORR (Objective response rate); OS (Overall survival); PBMC (Peripheral blood mononuclear cells); PC (Positive control); PET (Positron emission tomography); PI (Proteasome inhibitor); PD (Pharmacodynamic); PK (Pharmacokinetic); PFS (Progression-free Survival); PR (Partial response); PT (Prothrombin time); Q3W (Once every 3 weeks); Q4W (Once every 4 weeks); QTc (QT interval corrected for heart rate); RNA (Ribonucleic acid); RP2D (Recommended phase 2 dose); SAE (Serious adverse event); sCR (Stringent complete response); SIFE (Serum immunofixation electrophoresis); SMG (Safety monitoring group); SPEP (Serum protein electrophoresis); SUD (step-up dose); t_{1/2} (Terminal phase elimination half-life); T-BsAbs (T-cell engaging bispecific antibodies); TEAE (Treatment emergent adverse event); Tmax (Time to maximum observed serum concentration); Treg cells (Regulatory T cells); TTP (Time to progression); TTR (Time to response); UIFE (Urine immunofixation electrophoresis); ULN (Upper limit of normal); UPEP (Urine protein electrophoresis); US (United States); VI (Central compartment volume); VGPR (Very good partial response).

### 5.2 CD3×BCMA Bispecific Antibody

The present disclosure relates to methods of treating MM by administering to a subject a bispecific antibody that binds to CD3 (e.g., human CD3) and BCMA (e.g., human BCMA) (i.e., a CD3×BCMA bispecific antibody). In embodiments, a CD3×BCMA bispecific antibody used in the methods provided herein is etentamig (also known as ABBV-383 and TNB-383B). In embodiments, the CD3×BCMA bispecific antibody comprises: an anti-CD3 heavy chain variable (VH) domain that is paired with a light chain variable (VL) domain, wherein the VH domain and the VL domain together have binding affinity for CD3; a heavy chain variable domain having binding affinity to BCMA, in a bivalent configuration; and a variant human IgG4 Fc domain comprising a first heavy chain constant region sequence comprising an S228P mutation, an F234A mutation, an L235A mutation, and a T366W mutation (knob), and a second heavy chain constant region sequence comprising an S228P mutation, an F234A mutation, an L235A mutation, a T366S mutation, an L368A mutation, and a Y407V mutation (hole).

In embodiments, the CD3×BCMA bispecific antibody used in the various methods provided herein is etentamig. Etentamig is a human, monoclonal IgG4 bispecific antibody, comprising: (a) a first binding arm for human CD3, wherein the first binding arm comprises a heavy chain comprising the amino acid sequence of SEQ ID NO: 1; and a light chain comprising the amino acid sequence of SEQ ID NO:2; and (b) a second binding arm for human BCMA, wherein the second binding arm comprises: a heavy chain-only variable region in a bivalent configuration and comprising the amino acid sequence of SEQ ID NO:3 (Table 1).

TABLE 1
Etentamig CD3xBCMA Bispecific Antibody Sequences

| | |
|---|---|
| Anti-CD3 full | EIVMTQSPATLSVSPGERATLSCRASQSVSSNLAWYQQKPGQAPRLL |
| length light | IYGASTRATGIPARFSGSGSGTEFTLTISSLQSEDFAVYYCQQYNNW |
| chain (VL + | PWTFGQGTKVEIKRTVAAPSVFIFPPSDEQLKSGTASVVCLLNNFYP |
| kappa CL) | REAKVQWKVDNALQSGNSQESVTEQDSKDSTYSLSSTLTLSKADYEK |
| | HKVYACEVTHQGLSSPVTKSFNRGEC (SEQ ID NO: 1) |
| Anti-CD3 full | EVQLVESGGGLVQPGRSLRLSCAASGFTFDDYAMHWVRQAPGKGLEW |
| length heavy | VSGISWNSGSIGYADSVKGRFTISRDNAKNSLYLQMNSLRAEDTALY |
| chain (VH F2B + | YCAKDSRGYGDYRLGGAYWGQGTLVTVSSASTKGPSVFPLAPCSRST |
| silenced | SESTAALGCLVKDYFPEPVTVSWNSGALTSGVHTFPAVLQSSGLYSL |
| IgG4 Fc + | SSVVTVPSSSLGTKTYTCNVDHKPSNTKVDKRVESKYGPPCPPCPAP |
| knob (S228P, | EAAGGPSVFLFPPKPKDTLMISRTPEVTCVVVDVSQEDPEVQFNWYV |
| F234A, L235A; | DGVEVHNAKTKPREEQFNSTYRVVSVLTVLHQDWLNGKEYKCKVSNK |
| T366W)), with | GLPSSIEKTISKAKGQPREPQVYTLPPSQEEMTKNQVSLWCLVKGFY |
| C-terminal | PSDIAVEWESNGQPENNYKTTPPVLDSDGSFFLYSRLTVDKSRWQEG |
| Lysine (K) | NVFSCSVMHEALHNHYTQKSLSLSLGK (SEQ ID NO: 2) |
| BCMA bivalent | EVQLLESGGGLVQPGGSLRLSCAASGFTVSSYGMSWVRQAPGKGPEW |
| heavy chain | VSGIRGSDGSTYYADSVKGRFTISRDNSKNTLYLQMNSLRAEDTAVY |
| (Etentamig | YCAKQGENDGPFDHRGQGTLVTVSSGGGGSEVQLLESGGGLVQPGGS |
| with GS1) + | LRLSCAASGFTVSSYGMSWVRQAPGKGPEWVSGIRGSDGSTYYADSV |
| silenced IgG4 | KGRFTISRDNSKNTLYLQMNSLRAEDTAVYYCAKQGENDGPFDHRGQ |
| Fc hinge CH2 | GTLVTVSSESKYGPPCPPCPAPEAAGGPSVFLFPPKPKDTLMISRTP |
| CH3, hole | EVTCVVVDVSQEDPEVQFNWYVDGVEVHNAKTKPREEQFNSTYRVVS |
| (S228P, | VLTVLHQDWLNGKEYKCKVSNKGLPSSIEKTISKAKGQPREPQVYTL |
| F234A, L235A, | PPSQEEMTKNQVSLSCAVKGFYPSDIAVEWESNGQPENNYKTTPPVL |
| T366S, L368A, | DSDGSFFLVSRLTVDKSRWQEGNVFSCSVMHEALHNHYTQKSLSLSL |
| Y407V, with | GK (SEQ ID NO: 3) |
| C-terminal | |
| Lysine (K) | |
| Anti-CD3 full | EVQLVESGGGLVQPGRSLRLSCAASGFTFDDYAMHWVRQAPGKGLEW |
| length heavy | VSGISWNSGSIGYADSVKGRFTISRDNAKNSLYLQMNSLRAEDTALY |
| chain (VH F2B + | YCAKDSRGYGDYRLGGAYWGQGTLVTVSSASTKGPSVFPLAPCSRST |
| silenced | SESTAALGCLVKDYFPEPVTVSWNSGALTSGVHTFPAVLQSSGLYSL |
| IgG4 Fc + | SSVVTVPSSSLGTKTYTCNVDHKPSNTKVDKRVESKYGPPCPPCPAP |
| knob (S228P, | EAAGGPSVFLFPPKPKDTLMISRTPEVTCVVVDVSQEDPEVQFNWYV |
| F234A, L235A; | DGVEVHNAKTKPREEQFNSTYRVVSVLTVLHQDWLNGKEYKCKVSNK |
| T366W)), | GLPSSIEKTISKAKGQPREPQVYTLPPSQEEMTKNQVSLWCLVKGFY |
| without C- | PSDIAVEWESNGQPENNYKTTPPVLDSDGSFFLYSRLTVDKSRWQEG |
| terminal | NVFSCSVMHEALHNHYTQKSLSLSLG (SEQ ID NO: 4) |
| Lysine (K) | |
| BCMA bivalent | EVQLLESGGGLVQPGGSLRLSCAASGFTVSSYGMSWVRQAPGKGPEW |
| heavy chain | VSGIRGSDGSTYYADSVKGRFTISRDNSKNTLYLQMNSLRAEDTAVY |
| (Etentamig | YCAKQGENDGPFDHRGQGTLVTVSSGGGGSEVQLLESGGGLVQPGGS |
| with GS1) + | LRLSCAASGFTVSSYGMSWVROAPGKGPEWVSGIRGSDGSTYYADSV |
| silenced IgG4 | KGRFTISRDNSKNTLYLQMNSLRAEDTAVYYCAKQGENDGPFDHRGQ |
| Fc hinge CH2 | GTLVTVSSESKYGPPCPPCPAPEAAGGPSVFLFPPKPKDTLMISRTP |
| CH3, hole | EVTCVVVDVSQEDPEVQFNWYVDGVEVHNAKTKPREEQFNSTYRVVS |
| (S228P, | VLTVLHQDWLNGKEYKCKVSNKGLPSSIEKTISKAKGQPREPQVYTL |
| F234A, L235A, | PPSQEEMTKNQVSLSCAVKGFYPSDIAVEWESNGQPENNYKTTPPVL |
| T366S, L368A, | DSDGSFFLVSRLTVDKSRWQEGNVFSCSVMHEALHNHYTQKSLSLSL |
| Y407V, | G (SEQ ID NO: 5) |
| without C- | |
| terminal | |
| Lysine (K) | |

Thus, in embodiments of the methods provided herein, the CD3×BCMA bispecific antibody is etentamig and consists of a first polypeptide consisting of SEQ ID NO:1, a second polypeptide consisting of SEQ ID NO:2, and a third polypeptide consisting of SEQ ID NO:3. In embodiments, SEQ ID NO:2 of etentamig has the C-terminal lysine removed (SEQ ID NO:4). In embodiments, SEQ ID NO:3 of etentamig has the C-terminal lysine removed (SEQ ID NO:5). In embodiments, SEQ ID NO:2 and SEQ ID NO:3 of etentamig each have the C-terminal lysine removed (SEQ ID NO:4 and SEQ ID NO:5, respectively).

In embodiments, the CD3×BCMA bispecific antibody binds to human CD388. In embodiments, the CD3×BCMA bispecific antibody binds to human BCMA. In embodiments, the CD3×BCMA bispecific antibody binds to human CD388 and human BCMA. In embodiments, the CD3×BCMA bispecific antibody binds to human CD388 and binds to human BCMA. In embodiments, the CD3×BCMA bispecific antibody is a monoclonal antibody.

In embodiments, the CD3×BCMA bispecific antibody herein is formulated into pharmaceutical compositions comprising the CD3×BCMA bispecific antibody that are suitable for intravenous, IV bolus, or subcutaneous administration, collectively, injectable administration. The pharmaceutical compositions are generally formulated as sterile and in full compliance with all Good Manufacturing Practice (GMP) regulations of the U.S. Food and Drug Administration.

### 5.3 Methods of Treatment

Provided herein are embodiments of a method for treating MM in a human subject in need thereof, wherein the method comprises administering to the subject a step-up dose of a CD3×BCMA bispecific antibody and a maintenance dose of the CD3×BCMA bispecific antibody.

The antibodies described herein can be used for the treatment of B-cell related disorders, such as MM. MM is a B-cell malignancy characterized by a monoclonal expansion and accumulation of abnormal plasma cells in the bone marrow compartment. Current therapies for MM may cause remissions, but nearly all patients eventually relapse and die. There is substantial evidence of an immune-mediated elimination of myeloma cells in the setting of allogeneic hematopoietic stem cell transplantation; however, the toxicity of this approach is high, and few patients are cured. Although some monoclonal antibodies have shown promise for treating MM in preclinical studies and early clinical trials, consistent clinical efficacy of any monoclonal antibody therapy for MM has not been conclusively demonstrated. There is therefore a need for new therapies, including immunotherapies for MM (see, e.g., Carpenter et al., Clin Cancer Res 2013, 19 (8): 2048-2060). Overexpression or activation of BCMA by its proliferation-inducing ligand, APRIL it known to promote human MM progression in vivo. BCMA has also been shown to promote in vivo growth of xenografted MM cells harboring p53 mutation in mice. Because activity of the APRIL/BCMA pathway plays a central role in MM pathogenesis and drug resistance via bidirectional interactions between tumor cells and their supporting bone marrow microenvironment, BCMA has been identified as a target for the treatment of MM. For further details see, e.g., Yu-Tsu Tai et al., Blood 2016; 127 (25): 3225-3236.

In embodiments, the method for treating MM in a human subject comprises administering once about every four weeks to the subject as a monotherapy, a therapeutically effective amount of a maintenance dose of about 60 mg of a CD3×BCMA bispecific antibody comprising: (a) a first binding arm for human CD3, wherein the first binding arm comprises a heavy chain comprising the amino acid sequence of SEQ ID NO:1; and a light chain comprising the amino acid sequence of SEQ ID NO:2; and (b) a second binding arm for human BCMA, wherein the second binding arm comprises: a heavy chain-only variable region in a bivalent configuration and comprising the amino acid sequence of SEQ ID NO:3, thereby treating said MM. In embodiments, the CD3×BCMA bispecific antibody is etentamig.

In embodiments, the maintenance dose is 60 mg. In embodiments, the subject is administered a therapeutically effective amount of a maintenance dose of 60 mg of the CD3×BCMA bispecific antibody. In embodiments, the subject is administered a therapeutically effective amount of a maintenance dose of 60 mg of the CD3×BCMA bispecific antibody once every four weeks. In certain embodiments, the method does not require prior step-up dosing with the CD3×BCMA bispecific antibody (e.g., etentamig).

In embodiments, the method comprises treating relapsed or refractory MM in a human subject by administering a CD3×BCMA bispecific antibody. In embodiments, the subject previously has been exposed to at least one prior line of therapy. In embodiments, the subject has received at least two prior lines of therapy. In embodiments, the subject has received at least three prior lines of therapy. In another aspect, provided is a method of treating relapsed or refractory MM comprising administering a CD3×BCMA bispecific antibody as a fourth line therapy to patients who have previously been exposed to treatment with a PI, IMiD and an anti-CD38 mAb. In embodiments, the prior line of therapy includes treatment with a proteasome inhibitor (PI). In embodiments, the prior line of therapy includes treatment with an immunomodulatory imide (IMiD). In embodiments, the prior line of therapy includes treatment with an anti-CD38 monoclonal antibody (mAb) (e.g., daratumumab). In embodiments, the prior line of therapy includes treatment with a PI, an IMID and an anti-CD38 mAb (e.g., daratumumab). In embodiments, the CD3×BCMA bispecific antibody is a monoclonal antibody.

In embodiments, the CD3×BCMA bispecific antibody is administered intravenously. In embodiments, the CD3×BCMA bispecific antibody is administered subcutaneously.

In another aspect, provided herein is a method for treating MM in a human subject, comprising intravenously administering once every four weeks to the subject as a monotherapy, a therapeutically effective amount of a maintenance dose of 60 mg of etentamig, thereby treating said MM. In embodiments, MM is relapsed or refractory MM. In embodiments, the CD3×BCMA bispecific antibody is administered as a fourth line therapy to patients who have previously been exposed to treatment with a PI, IMiD and an anti-CD38 mAb. In embodiments, the CD3×BCMA bispecific antibody is administered as a third line therapy to patients who have previously been exposed to treatment with a PI, IMiD or an anti-CD38 mAb.

In embodiments, the CD3×BCMA bispecific antibody is administered for one or more cycles, wherein each cycle is about 4 weeks. In some embodiments, the CD3×BCMA bispecific antibody is administered for one cycle. In some embodiments, the CD3×BCMA bispecific antibody is administered for two cycles. In some embodiments, the CD3×BCMA bispecific antibody is administered for three cycles. In some embodiments, the CD3×BCMA bispecific antibody is administered for four cycles. In some embodiments, the CD3×BCMA bispecific antibody is administered for five cycles. In some embodiments, the CD3×BCMA bispecific antibody is administered for six cycles. In some embodiments, the CD3×BCMA bispecific antibody is administered for more than six cycles. In a specific embodiment, the CD3×BCMA bispecific antibody is etentamig.

In embodiments, the methods provided herein comprise administering a CD3×BCMA bispecific antibody as a monotherapy at a maintenance dose of 60 mg, administered once every 27-31 days, to a patient with MM. In some embodiments, the CD3×BCMA bispecific antibody is administered once every 27 days. In embodiments, the CD3×BCMA bispecific antibody is administered once every 28 days. In embodiments, the CD3×BCMA bispecific antibody is administered once every 29 days. In embodiments, the CD3×BCMA bispecific antibody is administered once every 30 days. In embodiments, the CD3×BCMA bispecific antibody is administered once every 31 days. In embodiments, the subject has received at least one prior line of therapy. In embodiments, the subject has received at least two prior lines of therapy. In embodiments, the subject has received at least three prior lines of therapy.

In embodiments, the methods provided herein comprise administering a CD3×BCMA bispecific antibody as a fourth line therapy at a maintenance dose of 60 mg, administered once every 4 weeks (28 days), to a patient with relapsed or refractory MM who has received at least three prior lines of therapy, including a PI, an IMiD and an anti-CD38 mAb.

Aspects of the disclosure include methods for evaluating the safety, pharmacokinetic (PK), pharmacodynamic (PD) and clinical activity of a CD3×BCMA bispecific antibody in subjects with relapsed or refractory MM who have received at least 3 prior lines of therapy, including a PI, an IMiD and an anti-CD38 mAb (e.g., daratumumab). “Line/regimen of therapy” is defined as a course of therapy (comprising at least 1 cycle) not interrupted by progressive disease, except in circumstances where the drug is not tolerated due to toxicity.

In embodiments, methods provided herein involve evaluating the safety, tolerability, PK and PD profiles of the CD3×BCMA bispecific antibody therapy (e.g., etentamig) administered once every four weeks (Q4W), in patients with relapsed/refractory MM who have received at least 3 prior lines of therapy, including a PI, an IMiD and an anti-CD38 mAb (e.g., daratumumab).

In embodiments, a CD3×BCMA bispecific antibody is initially administered as an IV infusion Q4W. Subjects may continue to receive the CD3×BCMA bispecific antibody as long as they do not meet any of the criteria for subject discontinuation, or until response-directed discontinuation.

In embodiments of the various methods provided herein, a subject is administered with one or more doses of one or more premedications that reduces a risk or severity of an immune-mediated toxicity prior to administering the CD3×BCMA bispecific antibody. In embodiments, the one or more premedications is selected from the group consisting of corticosteroids (e.g., dexamethasone, hydrocortisone, prednisolone, methylprednisolone), diphenhydramine, acetaminophen, ranitidine, famotidine, and any equivalents thereof, or any combination thereof. In embodiments, a subject is premedicated with dexamethasone (5-20 mg IV or orally) or equivalent, diphenhydramine (25 to 50 mg IV) or equivalent (e.g., Cetirizine 10 mg PO ×1), acetaminophen 650 to 1000 mg PO and ranitidine 150 mg PO/IV or equivalent about 15 to about 60 minutes prior to administration of the CD3×BCMA bispecific antibody infusion. In embodiments, premedicating a subject prior to administration of the CD3×BCMA bispecific antibody may reduce the risk and severity of immune-mediated toxicities commonly observed with mAb therapy (e.g., CRS and/or ICANS). In embodiments, reducing the risk and severity of immune-medicated toxicities commonly observed with mAb therapy contributes to “safely” treating a disease in a human. In embodiments, a subject is administered acetaminophen every six hours for the first 24 hours prior to infusion of the CD3×BCMA bispecific antibody. In embodiments, a subject is administered diphenhydramine and famotidine 15 to 60 minutes prior to infusion of the CD3×BCMA bispecific antibody. In embodiments, a subject is administered diphenhydramine and famotidine orally 15 to 60 minutes prior to infusion of the CD3×BCMA bispecific antibody. In embodiments, a subject is administered diphenhydramine and famotidine IV 15 to 60 minutes prior to infusion of the CD3×BCMA bispecific antibody.

In embodiments, a subject is administered dexamethasone before administration of the CD3×BCMA bispecific antibody (i.e., premedicated with dexamethasone). In embodiments, the CD3×BCMA bispecific antibody is etentamig. In embodiments, the total amount of dexamethasone administered in a first cycle is about 40-50 mg. In embodiments, the total amount of dexamethasone administered in a first cycle is 46 mg. In embodiments, the total amount is about 15 mg. In embodiments, the total amount is about 16 mg. In embodiments, the total amount is about 17 mg. In embodiments, the total amount is about 18 mg. In some embodiments, the total amount is about 19 mg. In embodiments, the total amount is about 20 mg. In embodiments, the total amount is about 21 mg. In embodiments, the total amount is about 22 mg. In embodiments, the total amount is about 23 mg. In embodiments, the total amount is about 24 mg. In embodiments, the total amount is about 25 mg. In embodiments, the total amount is about 26 mg. In embodiments, the total amount is about 27 mg. In embodiments, the total amount is about 28 mg. In embodiments, the total amount is about 29 mg. In embodiments, the total amount is about 30 mg. In embodiments, the total amount is about 31 mg. In embodiments, the total amount is about 32 mg. In embodiments, the total amount is about 33 mg. In embodiments, the total amount is about 34 mg. In embodiments, the total amount is about 35 mg. In embodiments, the total amount is about 36 mg. In embodiments, the total amount is about 37 mg. In embodiments, the total amount is about 38 mg. In embodiments, the total amount is about 39 mg. In embodiments, the total amount is about 40 mg. In embodiments, the subject is premedicated with dexamethasone only before administration of the CD3×BCMA bispecific antibody in the first cycle. In embodiments, the subject is not premedicated with dexamethasone before administration of the CD3×BCMA bispecific antibody in subsequent cycles. In embodiments, the total amount of dexamethasone administered as a pre-medication to the subject is tapered off in the subsequent cycles.

In embodiments, a subject is administered dexamethasone about 15 to about 60 minutes before administration of the CD3×BCMA bispecific antibody. In embodiments, a subject is administered a premedication dose of about 10 mg dexamethasone about 15 to about 60 minutes before administration of the CD3×BCMA bispecific antibody. In embodiments, the CD3×BCMA bispecific antibody is administered at a maintenance dose of 60 mg of etentamig. In embodiments, the CD3×BCMA bispecific antibody is administered at a step-up dose of 2 mg of etentamig In embodiments, the CD3×BCMA antibody is etentamig. In embodiments, etentamig is administered IV.

In embodiments, a patient is administered a modified premedication dose regimen (“MPDR”), including (a) one administration of dexamethasone about 12 hours prior to administration of a maintenance dose of the CD3×BCMA bispecific antibody, (b) one administration of dexamethasone about 3 to 4 hours prior to administration of the maintenance dose of the CD3×BCMA bispecific antibody, and (c) one administration of dexamethasone about 15 to about 60 minutes prior to administration of the maintenance dose of the CD3×BCMA bispecific antibody. In embodiments, a patient is administered a premedication dose regimen, including (a) one administration of about 8 mg dexamethasone about 12 hours prior to administration of the maintenance dose of the CD3×BCMA bispecific antibody, (b) one administration of about 8 mg dexamethasone about 3 to 4 hours prior to administration of the maintenance dose of the CD3×BCMA bispecific antibody, and (c) one administration of about 20 mg dexamethasone about 15 to about 60 minutes prior to administration of the maintenance dose of the CD3×BCMA bispecific antibody, where the maintenance dose of the CD3×BCMA bispecific antibody is administered about every 4 weeks. In a specific embodiment, the CD3×BCMA bispecific antibody is etentamig. In embodiments, etentamig is administered IV.

In embodiments, the first administration of dexamethasone in the MPDR is administered orally. In embodiments, the second administration of dexamethasone in the MPDR is administered orally. In embodiments, the first and second administrations of dexamethasone in the MPDR are administered orally. In embodiments, the third administration of dexamethasone in the MPDR is administered orally. In embodiments, one administration of dexamethasone in the MPDR is administered intravenously. In embodiments, all administrations of dexamethasone in the MPDR are administered orally. In embodiments, a first and second administration of dexamethasone in the MPDR are administered orally, and a third administration of dexamethasone in the MPDR is administered intravenously.

In some embodiments, the amount of dexamethasone administered prior to the maintenance dose of etentamig is tapered in subsequent cycles if no CRS event occurred in a prior cycle. In embodiments, the methods provided herein comprise administering about 20 mg dexamethasone about 15 to about 60 minutes prior to administration of the maintenance dose of the CD3×BCMA bispecific antibody in the first cycle and administering about 10 mg dexamethasone prior to administration of the CD3×BCMA bispecific antibody in a subsequent cycle, wherein the CD3×BCMA bispecific antibody is administered as a monotherapy at a maintenance dose of about 60 mg once about every 4 weeks. In embodiments, the methods provided herein comprise administering about 10 mg dexamethasone prior to administration of the CD3×BCMA bispecific antibody, and administering about 5 mg dexamethasone prior to administration of the CD3×BCMA bispecific antibody in a subsequent cycle, wherein the CD3×BCMA bispecific antibody is administered as a monotherapy at a maintenance dose of about 60 mg once about every 4 weeks. In embodiments, the methods provided herein comprise administering about 5 mg dexamethasone prior to administration of the CD3×BCMA bispecific antibody, and administering about 10 mg dexamethasone prior to administration of the CD3×BCMA bispecific antibody in a subsequent cycle, wherein the CD3×BCMA bispecific antibody is administered as a monotherapy at a maintenance dose of about 60 mg once about every 4 weeks. In embodiments, the methods provided herein comprise administering about 5 mg dexamethasone prior to administration of the CD3×BCMA bispecific antibody, and omitting dexamethasone as a premedication prior to administration of the CD3×BCMA bispecific antibody in a subsequent cycle, wherein the CD3×BCMA bispecific antibody is administered as a monotherapy at a maintenance dose of about 60 mg once about every 4 weeks. In a specific embodiment, the CD3×BCMA bispecific antibody is etentamig. In embodiments, etentamig is administered IV.

In embodiments, the first maintenance dose of the CD3×BCMA bispecific antibody infusion is given over 2 hours (+10 minutes). If no infusion reactions occur during the first maintenance dose of the CD3×BCMA bispecific antibody, the duration of infusion for subsequent doses of the CD3×BCMA bispecific antibody may be shortened, in embodiments. In embodiments, a patient is monitored for 4 hours after the first infusion, and for 2 hours after each subsequent infusion. In embodiments, the 4 hours of close observation post-infusion in the first cycle should occur during the subject's hospitalization after the first maintenance dose of the CD3×BCMA bispecific antibody. In embodiments, a subject is hospitalized for a total of 48 hours, from day 1 to day 3, following the first maintenance dose of the CD3×BCMA bispecific antibody.

In embodiments, the method for treating MM in a human subject in need thereof further comprises administering to the subject a step-up dose of a CD3×BCMA bispecific antibody prior to the maintenance dose of the CD3×BCMA bispecific antibody.

In embodiments, the method for treating MM in a human subject in need thereof comprises administering to the subject a step-up dose of etentamig and administering to the subject a maintenance dose of etentamig. In embodiments, the method comprises administering to the subject a step-up dose of etentamig of about 2 mg; and administering to the subject a maintenance dose of etentamig of about 60 mg per cycle, wherein each cycle comprises about four weeks. In embodiments, the step-up dose of etentamig is only administered in the first cycle.

In embodiments, the method for treating MM in a human subject in need thereof comprises administering a modified premedication dose regimen prior to the maintenance dose of etentamig. In embodiments, the premedication is a corticosteroid. In embodiments, the corticosteroid is selected from the group consisting of dexamethasone, methylprednisolone, prednisolone, and hydrocortisone. In embodiments, the premedication is an agent selected from the group consisting of dexamethasone, diphenhydramine, acetaminophen, ranitidine, famotidine, and any equivalents thereof, or any combination thereof. In embodiments, a patient is administered a corticosteroid before administration of the maintenance dose of about 60 mg of etentamig. In embodiments, a patient is administered dexamethasone before administration of the maintenance dose of about 60 mg of etentamig.

In embodiments, the dexamethasone is administered IV. In embodiments, the dexamethasone is administered orally. In embodiments, the dexamethasone is administered both IV and orally.

In embodiments, a patient is administered on day 1 of a cycle 10 mg of a corticosteroid about 15 to about 60 minutes before administration of a step-up dose of etentamig, and then on days 3-4 of a cycle further administered 36 mg corticosteroid before administration of the maintenance dose of etentamig. In embodiments, 36 mg corticosteroid is administered as a modified premedication dose regimen: (i) 8 mg corticosteroid about 12 to about 16 hours before administration of a maintenance dose of etentamig, (ii) 8 mg corticosteroid about 2 to about 5 hours before administration of the maintenance dose of etentamig, and (iii) 20 mg corticosteroid about 15 to about 60 minutes before administration of a maintenance dose of etentamig. In embodiments, the step-up dose of etentamig is 2 mg, and the maintenance dose of etentamig is 60 mg. In embodiments, the corticosteroid is selected from the group consisting of dexamethasone, methylprednisolone, prednisolone, and hydrocortisone.

In embodiments, on day 1 of a cycle, a subject is administered 10 mg dexamethasone about 15 to about 60 minutes before administration of a step-up dose comprising 2 mg of etentamig, and then on days 3-4 of a cycle, the subject is further administered 36 mg dexamethasone before administration of the maintenance dose of etentamig. In embodiments, the 36 mg dexamethasone is administered as a modified premedication dose regimen: (i) 8 mg dexamethasone about 12 to about 16 hours before administration of a maintenance dose of etentamig, (ii) 8 mg dexamethasone about 2 to about 5 hours before administration of the maintenance dose of etentamig, and (iii) 20 mg dexamethasone about 15 to about 60 minutes before administration of a maintenance dose of etentamig. In embodiments, the step-up dose of etentamig is 2 mg, and the maintenance dose of etentamig is 60 mg.

In embodiments, the total amount of dexamethasone administered in the first cycle is about 10 to 60 mg. In embodiments, the total amount of dexamethasone administered in the first cycle is about 20 to 60 mg. In embodiments, the total amount of dexamethasone administered in the first cycle is about 20 to 50 mg. In embodiments, the total amount of dexamethasone administered in the first cycle is about 40 to 50 mg. In embodiments, the total amount of dexamethasone administered in the first cycle is about 50 to 60 mg. In embodiments, the total amount of dexamethasone administered in the first cycle is about 46 mg. In embodiments, the total amount of dexamethasone administered in the first cycle is about 56 mg.

In embodiments, the total amount of dexamethasone administered in subsequent cycles following the first cycle is about 30 to 40 mg. In embodiments, the total amount of dexamethasone administered in the subsequent cycles following the first cycle is about 36 mg. In embodiments, administered prior to the maintenance dose of etentamig is tapered in subsequent cycles if no CRS event occurred in a prior cycle.

In embodiments, the dexamethasone is administered IV about 15 to about 60 minutes before administration of the step-up dose of etentamig. In embodiments, the dexamethasone is administered orally about 15 to about 60 minutes before administration of the step-up dose of etentamig. In embodiments, the dexamethasone is administered orally about 12 to about 16 hours before administration of a maintenance dose of etentamig. In embodiments, the dexamethasone is administered orally about 2 to about 5 hours before administration of the maintenance dose of etentamig. In embodiments, the dexamethasone is administered IV or orally about 15 to about 60 minutes before administration of the maintenance dose of etentamig. In embodiments, etentamig is administered IV. In embodiments, the cycle is about four weeks.

In embodiments, a patient is administered on day 1 of a first cycle 10 mg dexamethasone about 15 to about 60 minutes before administration of a step-up dose of 2 mg of etentamig and then on days 3-4 of a cycle further administered 36 mg dexamethasone through a MPDR before administration of the maintenance dose of etentamig. In embodiments, 36 mg dexamethasone is administered through a MPDR as: (i) 8 mg dexamethasone about 12 to about 16 hours before administration of a maintenance dose of etentamig, (ii) 8 mg dexamethasone about 2 to about 5 hours before administration of the maintenance dose of etentamig, and (iii) 20 mg dexamethasone about 15 to about 60 minutes before administration of a maintenance dose of etentamig.

In embodiments, a patient is administered on day 1 of a first cycle, an oral premedication dose of about 10 mg dexamethasone, followed by a step-up dose of about 2 mg of etentamig. Then, on day 3 (or up to day 13), the patient is administered an oral premedication dose of about 8 mg of dexamethasone. Then, on day 4, (or up do day 14), the patient is administered an oral premedication dose of about 8 mg of dexamethasone, followed by an oral or intravenous premedication dose of about 20 mg of dexamethasone, further followed by a maintenance dose of about 60 mg of etentamig. On day 1 of one or more subsequent cycles, the patient is administered the maintenance dose of etentamig.

In embodiments, the patient is administered on day 1 of a first cycle, an oral premedication dose of about 10 mg dexamethasone, followed by a step-up dose of about 2 mg of etentamig. Then, on day 4, the patient is administered an oral premedication dose of about 8 mg of dexamethasone. Then, on day 5, the patient is administered an oral premedication dose of about 8 mg of dexamethasone, followed by an oral or intravenous premedication dose of about 20 mg of dexamethasone, further followed by a maintenance dose of about 60 mg of etentamig. On day 1 of one or more subsequent cycles following cycle 1, the patient is administered the maintenance dose of etentamig.

In embodiments, the patient is administered on day 1 of a first cycle, an oral premedication dose of about 10 mg dexamethasone, followed by a step-up dose of about 2 mg of etentamig. Then, on day 5, the patient is administered an oral premedication dose of about 8 mg of dexamethasone. Then, on day 6, the patient is administered an oral premedication dose of about 8 mg of dexamethasone, followed by an oral or intravenous premedication dose of about 20 mg of dexamethasone, further followed by a maintenance dose of about 60 mg of etentamig. On day 1 of one or more subsequent cycles, the patient is administered the maintenance dose of etentamig.

In embodiments, the patient is administered on day 1 of a first cycle, an oral premedication dose of about 10 mg dexamethasone, followed by a step-up dose of about 2 mg of etentamig. Then, on day 6, the patient is administered an oral premedication dose of about 8 mg of dexamethasone. Then, on day 7, the patient is administered an oral premedication dose of about 8 mg of dexamethasone, followed by an oral or intravenous premedication dose of about 20 mg of dexamethasone, further followed by a maintenance dose of about 60 mg of etentamig. On day 1 of one or more subsequent cycles, the patient is administered the maintenance dose of etentamig.

In embodiments, the patient is administered on day 1 of a first cycle, an oral premedication dose of about 10 mg dexamethasone, followed by a step-up dose of about 2 mg of etentamig. Then, on day 7, the patient is administered an oral premedication dose of about 8 mg of dexamethasone. Then, on day 8, the patient is administered an oral premedication dose of about 8 mg of dexamethasone, followed by an oral or intravenous premedication dose of about 20 mg of dexamethasone, further followed by a maintenance dose of about 60 mg of etentamig. On day 1 of one or more subsequent cycles, the patient is administered the maintenance dose of etentamig.

In embodiments, the patient is administered on day 1 of a first cycle, an oral premedication dose of about 10 mg dexamethasone, followed by a step-up dose of about 2 mg of etentamig. Then, on day 8, the patient is administered an oral premedication dose of about 8 mg of dexamethasone. Then, on day 9, the patient is administered an oral premedication dose of about 8 mg of dexamethasone, followed by an oral or intravenous premedication dose of about 20 mg of dexamethasone, further followed by a maintenance dose of about 60 mg of etentamig. On day 1 of one or more subsequent cycles, the patient is administered the maintenance dose of etentamig.

In embodiments, the patient is administered on day 1 of a first cycle, an oral premedication dose of about 10 mg dexamethasone, followed by a step-up dose of about 2 mg of etentamig. Then, on day 9, the patient is administered an oral premedication dose of about 8 mg of dexamethasone. Then, on day 10, the patient is administered an oral premedication dose of about 8 mg of dexamethasone, followed by an oral or intravenous premedication dose of about 20 mg of dexamethasone, further followed by a maintenance dose of about 60 mg of etentamig. On day 1 of one or more subsequent cycles, the patient is administered the maintenance dose of etentamig.

In embodiments, the patient is administered on day 1 of a first cycle, an oral premedication dose of about 10 mg dexamethasone, followed by a step-up dose of about 2 mg of etentamig. Then, on day 10, the patient is administered an oral premedication dose of about 8 mg of dexamethasone. Then, on day 11, a patient is administered an oral premedication dose of about 8 mg of dexamethasone, followed by an oral or intravenous premedication dose of about 20 mg of dexamethasone, further followed by a maintenance dose of about 60 mg of etentamig. On day 1 of one or more subsequent cycles, the patient is administered the maintenance dose of etentamig.

In embodiments, the patient is administered on day 1 of a first cycle, an oral premedication dose of about 10 mg dexamethasone, followed by a step-up dose of about 2 mg of etentamig. Then, on day 11, the patient is administered an oral premedication dose of about 8 mg of dexamethasone. Then, on day 12, the patient is administered an oral premedication dose of about 8 mg of dexamethasone, followed by an oral or intravenous premedication dose of about 20 mg of dexamethasone, further followed by a maintenance dose of about 60 mg of etentamig. On day 1 of one or more subsequent cycles, the patient is administered the maintenance dose of etentamig.

In embodiments, the patient is administered on day 1 of a first cycle, an oral premedication dose of about 10 mg dexamethasone, followed by a step-up dose of about 2 mg of etentamig. Then, on day 12, the patient is administered an oral premedication dose of about 8 mg of dexamethasone. Then, on day 13, the patient is administered an oral premedication dose of about 8 mg of dexamethasone, followed by an oral or intravenous premedication dose of about 20 mg of dexamethasone, further followed by a maintenance dose of about 60 mg of etentamig. On day 1 of one or more subsequent cycles, the patient is administered the maintenance dose of etentamig.

In embodiments, the patient is administered on day 1 of a first cycle, an oral premedication dose of about 10 mg dexamethasone, followed by a step-up dose of about 2 mg of etentamig. Then, on day 13, the patient is administered an oral premedication dose of about 8 mg of dexamethasone. Then, on day 14, the patient is administered an oral premedication dose of about 8 mg of dexamethasone, followed by an oral or intravenous premedication dose of about 20 mg of dexamethasone, further followed by a maintenance dose of about 60 mg of etentamig. On day 1 of one or more subsequent cycles, the patient is administered the maintenance dose of etentamig.

In embodiments, on day 15 or later of a cycle, if the patient has not yet received administration of a maintenance dose of etentamig (e.g., if administration of a maintenance dose has been delayed beyond day 14 of a cycle), then the patient is administered 10 mg dexamethasone about 15 to about 60 minutes before administration of a step-up dose of 2 mg of etentamig (a “repeat step-up dose”). Three days following the repeat step-up dose, the patient is administered 36 mg dexamethasone via a MPDR before administration of the maintenance dose of etentamig. In embodiments, 36 mg dexamethasone is administered by a MPDR as: (i) 8 mg dexamethasone about 12 to about 16 hours before administration of a maintenance dose of etentamig, (ii) 8 mg dexamethasone about 2 to about 5 hours before administration of the maintenance dose of etentamig, and (iii) 20 mg dexamethasone about 15 to about 60 minutes before administration of a maintenance dose of etentamig. In embodiments, the step-up dose of etentamig is 2 mg, and the maintenance dose of etentamig is 60 mg.

In embodiments, on day 1 of cycle 2, the patient is administered 10 mg dexamethasone about 15 to about 60 minutes before administration of the maintenance dose of etentamig. In embodiments, on day 1 of cycle 3, the patient is administered 5 mg dexamethasone about 15 to about 60 minutes before administration of the maintenance dose of etentamig. In embodiments, the maintenance dose of etentamig is 60 mg.

Embodiments of the present disclosure further relate to a method for treating MM in a human subject, wherein the method comprises (1) a first cycle comprising administering a step-up dose of etentamig and then further administering a maintenance dose of etentamig, and (2) one or more additional cycles comprising administering the maintenance dose of etentamig. In embodiments, each cycle is 27-31 days. In embodiments, each cycle is 27 days. In embodiments, each cycle is 28 days. In embodiments, each cycle is 29 days. In embodiments, each cycle is 30 days. In embodiments, each cycle is 31 days.

In embodiments, the step-up dose of the CD3×BCMA bispecific antibody is administered for one cycle. In embodiments, the maintenance dose of the CD3×BCMA bispecific antibody is administered for one or more cycles, wherein each cycle is about 4 weeks.

In embodiments, on day 1 of cycle 2, the patient is administered 10 mg of a corticosteroid about 15 to about 60 minutes before administration of the step-up dose of the CD3×BCMA bispecific antibody. In embodiments, on day 1 of cycle 2, the patient is administered 10 mg dexamethasone about 15 to about 60 minutes before administration of the maintenance dose of about 60 mg of etentamig.

In embodiments, the patient is premedicated with dexamethasone only before administration of etentamig in the first cycle. In embodiments, the patient is not premedicated with dexamethasone before administration of etentamig in the second or later (subsequent) cycles. In embodiments, the total amount of dexamethasone administered as a pre-medication to the patient is tapered off in the second or later cycles.

In embodiments, the patient is administered (a) a first premedication dose of dexamethasone about 15 to about 60 minutes prior to administration of a step-up dose comprising 2 mg of etentamig, (b) a second premedication dose of dexamethasone about 12 to 16 hours prior to administration of a maintenance dose of etentamig, (c) a third premedication dose of dexamethasone about 2 to 5 hours prior to administration of the maintenance dose of etentamig, and (d) a fourth premedication dose of dexamethasone about 15 to about 60 minutes prior to administration of the maintenance dose of etentamig. In embodiments, the patient is administered (a) a first premedication dose of about 10 mg dexamethasone about 15 to about 60 minutes prior to administration of a step-up dose of etentamig (e.g., 2 mg of etentamig), (b) a second premedication dose of about 8 mg dexamethasone about 12 to 16 hours prior to administration of a maintenance dose of etentamig, (c) a third premedication dose of about 8 mg dexamethasone about 2 to 5 hours prior to administration of the maintenance dose of etentamig, and (d) a fourth premedication dose of about 20 mg dexamethasone about 15 to about 60 minutes prior to administration of the maintenance dose of etentamig, where the maintenance dose of etentamig is administered as a monotherapy at a maintenance dose of about 60 mg once about every 4 weeks. In embodiments, etentamig is administered IV. In embodiments, the first premedication dose of dexamethasone is administered intravenously. In embodiments, the second premedication dose of dexamethasone is administered orally. In embodiments, the third premedication dose of dexamethasone is administered orally. In embodiments, the second and third premedication doses of dexamethasone are administered orally. In embodiments, the fourth premedication of dexamethasone is administered orally. In embodiments, the fourth premedication dose of dexamethasone is administered intravenously. In embodiments, each dose of dexamethasone is administered orally. In embodiments, the first dose of dexamethasone is administered intravenously, the second and third doses of dexamethasone are administered orally, and the fourth dose of dexamethasone is administered intravenously.

In embodiments, the amount of dexamethasone is tapered in subsequent cycles if no CRS event occurred in a prior cycle. In embodiments, the dexamethasone is not administered if no CRS event occurred in a prior cycle. In embodiments, the dexamethasone is not administered after the first cycle. In embodiments, the dexamethasone is tapered after the first cycle, and not administered after the second cycle.

In embodiments, the total amount of dexamethasone administered in the first cycle is about 46 mg (e.g., 10 mg dexamethasone prior to administration of a step-up dose of etentamig and 36 mg dexamethasone administered via a MPDR prior to administration of a maintenance dose of etentamig), and the total amount of dexamethasone administered in a subsequent cycle is about 10 mg or less, wherein each cycle is about every 4 weeks. In embodiments, the total amount of dexamethasone administered in the first cycle is about 46 mg, and the total amount of dexamethasone administered in a subsequent cycle is about 5 mg, wherein each cycle is about every 4 weeks. In embodiments, the total amount of dexamethasone administered in the first cycle is about 46 mg, the total amount of dexamethasone administered in the second cycle is about 5 mg, and dexamethasone is not administered in subsequent cycles after the second cycle, wherein each cycle is about every 4 weeks. In embodiments, the total amount of dexamethasone administered in the first cycle is about 46 mg, and dexamethasone is not administered in subsequent cycles, wherein each cycle is about every 4 weeks.

In embodiments, the total amount of dexamethasone administered in the first cycle is about 56 mg (e.g., 10 mg dexamethasone prior to administration of a step-up dose of the CD3×BCMA bispecific antibody, 10 mg dexamethasone prior to administration of a repeat step-up dose of the CD3×BCMA bispecific antibody, and 36 mg dexamethasone administered via a MPDR prior to administration of a maintenance dose of the CD3×BCMA bispecific antibody), and the total amount of dexamethasone administered in a subsequent cycle is about 10 mg or less, wherein each cycle is about every 4 weeks. In embodiments, the total amount of dexamethasone administered in the first cycle is about 56 mg, and the total amount of dexamethasone administered in a subsequent cycle is about 5 mg, wherein each cycle is about every 4 weeks. In embodiments, the total amount of dexamethasone administered in the first cycle is about 56 mg, the total amount of dexamethasone administered in the second cycle is about 5 mg, and dexamethasone is not administered in subsequent cycles after the second cycle, wherein each cycle is about every 4 weeks. In embodiments, the total amount of dexamethasone administered in the first cycle is about 56 mg, and dexamethasone is not administered in subsequent cycles, wherein each cycle is about every 4 weeks. In an embodiment, the CD3×BCMA bispecific antibody is etentamig. In embodiments, etentamig is administered IV.

In embodiments, the step-up dose of the CD3×BCMA bispecific antibody infusion is given as an IV bolus. In embodiments, the first maintenance dose of the CD3×BCMA bispecific antibody infusion is given over 2 hours (+10 minutes). If no infusion reactions occur during the first maintenance dose of the CD3×BCMA bispecific antibody, the duration of infusion for subsequent doses of the CD3×BCMA bispecific antibody may be shortened. In embodiments, a patient is monitored for 4 hours after each dose of the CD3×BCMA bispecific antibody in the first cycle, and for 2 hours after each subsequent infusion of the CD3×BCMA bispecific antibody. In embodiments, the 4 hours of close observation post-infusion(s) in the first cycle should occur during the patient's hospitalization after each dose of the CD3×BCMA bispecific antibody. In embodiments, a patient may be hospitalized for 24 to 48 hours following each dose of the CD3×BCMA bispecific antibody during the first cycle.

It will be understood that in any of the aspects and embodiments of the various methods provided herein, the CD3×BCMA bispecific antibody is etentamig, a human, monoclonal IgG4 bispecific antibody, comprising: (a) a first binding arm for human CD3, wherein the first binding arm comprises a heavy chain comprising the amino acid sequence of SEQ ID NO:1; and a light chain comprising the amino acid sequence of SEQ ID NO:2; and (b) a second binding arm for human BCMA, wherein the second binding arm comprises: a heavy chain-only variable region in a bivalent configuration and comprising the amino acid sequence of SEQ ID NO:3. In embodiments, etentamig is administered IV. In embodiments, etentamig is administered as a monotherapy.

5.4 Exemplary Subject Screening and Assessments

Certain aspects of the examples and other disclosure provided herein utilize the materials and methods below.

5.4.1 Exemplary Treatment Inclusion and Exclusion Criteria

In some embodiments, patients undergo screening procedures within 28 days prior to initial drug administration. In embodiments, adult subjects who meet the inclusion criteria and who do not meet any of the exclusion criteria are eligible for treatment.

### Exemplary Inclusion Criteria:

1. Subject must be ≥18 years of age.
2. Three or more prior lines of therapy with exposure to a PI, and IMID, and an anti-CD38 antibody (e.g., daratumumab). In order to be eligible for this study subjects must not be candidates for treatment regimens known to provide clinical benefit in MM.
3. Subject has an Eastern Cooperative Oncology Group (ECOG) Performance Status of ≤2.
4. Subject is capable of understanding and complying with parameters as outlined in the protocol and able to sign informed consent.
5. Subject must have adequate bone marrow function, defined as: absolute neutrophil count (ANC) ≥1000/mm³; platelets ≥50,000/mm³; hemoglobin ≥8.0 g/dL. Transfusion and/or growth factor support is permitted prior to assessment, but neutrophils, platelets, and hemoglobin must be stable for at least 72 hours after transfusion and/or growth factor administration prior to screening for the subject to be eligible.
6. Subject must have an eGFR ≥30 mL/min as estimated by the MDRD formula.
7. Subject must have total bilirubin≤1.5 × upper limit of normal (ULN; except if the subject has a known diagnosis of Gilbert's syndrome, in which case bilirubin must be <3 × ULN), aspartate aminotransferase (AST) and alanine aminotransferase (ALT)≤3 × ULN.
8. Serum calcium (corrected for albumin) at or below the ULN range (subject may enroll in the setting of hypercalcemia IF hypercalcemia resolves with standard treatment by Cycle 1, Day 1) prior to study therapy initiation.
9. If female, subject must be either postmenopausal (for at least 12 consecutive months), OR permanently surgically sterile, OR for women of childbearing potential practicing at least 1 protocol specified method of female birth control AND one method of male birth control (vasectomy or condoms), starting at Cycle 1, Day 1 through at least 6 months after the last dose of study drug.
10. Measurable Disease: Subject has a diagnosis of MM and documented prior treatment with a PI, an IMiD (e.g., pomalidomide, lenalidomide), and an anti-CD38 mAb therapy (e.g., daratumumab) as part of 3 or more lines of therapy. There is no maximum number of prior regimens and prior bone marrow transplant is acceptable subject is >12 weeks (autologous) or >1 year (allogeneic) status-post transplantation. In order to be eligible for this study subjects must not be candidates for treatment regimens known to provide clinical benefit in MM.
   Measurable disease is defined as at least 1 of the following:
Serum M-protein ≥0.5 g/dL (≥5 g/L)
Urine M-protein ≥200 mg/24h
Serum free light chain (FLC) assay: Involved FLC level ≥10 mg/dl (≥100 mg/L) and an abnormal serum FLC ratio (<0.26 or >1.65).
11. Subject has confirmed evidence of relapse/progression from the immediately prior MM therapy, or subject is relapsed/refractory to the immediate prior MM therapy. (′Relapsed/refractory′ is defined as subjects with a history of minimal response [MR] or better response to prior therapy, now with disease progression within 60 days of the last therapy. ‘Relapse’ is defined as previously treated myeloma that progresses and requires initiation of salvage therapy, without meeting International Myeloma Working Group [IMWG] uniform response criteria for relapsed/refractory.)
12. Subject has adequate archival tumor bone marrow sample if available or consents to a fresh pre-treatment bone marrow tumor biopsy.

### Exemplary Exclusion Criteria

In embodiments, a subject will not be eligible for treatment if he/she meets any of the following criteria:
1. Subject has been diagnosed or treated for another malignancy within 3 years of enrollment, with the exception of basal cell or squamous cell carcinoma of the skin, in situ malignancy, low-risk prostate carcinoma after curative therapy, or complete resection/curative therapy of an advanced malignancy.
2. Subject has a history of CNS involvement by their myeloma.
3. Subject has a history of >Grade 3 peripheral neuropathy.
4. Subject has a history of plasma cell leukemia, POEMS syndrome, or amyloidosis.
5. Subject has received another investigational drug within 21 days of enrollment.
6. Subject has ever received BCMA-targeted therapy. Subjects who have received targeted therapy against non-BCMA targets will not be excluded.
7. Subject has received a peripheral autologous stem cell transplant within 12 weeks, or an allogeneic transplant within 1 year of the first dose of study drug treatment.
8. Subject has any medical or psychiatric condition which in the opinion of the investigator or Study Medical Monitor places the subject at an unacceptably high risk for toxicities, could interfere with successful or safe delivery of therapy, or could interfere with evaluation of the investigational product or interpretation of subject safety or study results. Examples include history of significant mucosal/internal bleeding, major psychiatric illness, drug abuse (including active alcoholism), or known allergy or hypersensitivity to components of the study drug formulation.
9. Subject has received any therapy to treat cancer (including radiation, chemotherapy, biologics, cellular therapies, and/or steroids at doses >20 mg) or undergone a major surgical procedure within 21 days, or within 5 half-lives of an anticancer drug, prior to the first dose of study treatment, whichever is shorter.
10. Subject has known active infection requiring parenteral anti-infective treatment. Upon completion of antibiotics and resolution of symptoms, the subject is considered eligible for the study from an infection standpoint.
11. Confirmed positive test results for human immunodeficiency virus (HIV), or subjects with chronic or active hepatitis B virus (HBV) or hepatitis C virus (HCV). Subjects who have a history of HBV or HCV who have documented cures (HBV: HBsAg negative; HCV: undetectable CV ribonucleic acid (RNA) 24 weeks after the end of treatment) may be enrolled.
12. Major cardiac abnormalities, such as but not limited to the following: uncontrolled angina or unstable life-threatening arrhythmias, history of myocardial infarction <12 weeks before Screening, Class >3 New York Heart Association congestive heart failure, severe cardiac insufficiency, or persistent QTc prolongation (>480 msec, QTc Fridericia).
13. Subject has unresolved AEs >Grade 2 (National Cancer Institute (NCI) Common Terminology Criteria for Adverse Events (CTCAE] v5.0) from prior anticancer therapy except for:
Alopecia.
Peripheral neuropathy (peripheral neuropathy >Grade 3 will be excluded).
Anemia or thrombocytopenia (thrombocytopenia must be Grade 4 to trigger exclusion, Grade 3 with symptoms or bleeding, respectively, or return within 72 hours despite transfusion support).
Subjects with irreversible toxicity that is not reasonably expected to be exacerbated by any of the investigational products may be included (e.g., hearing loss) after consultation with the Study Medical Monitor.

### 5.4.2 Eastern Cooperative Oncology Group (ECOG) Performance Status

Aspects of the disclosure may include assessing an ECOG performance status of a subject at Screening, Day 1 of each cycle prior to dosing, at the EOT Visit or upon subject discontinuation, and at the 90-day follow-up visit after the last dose of the CD3×BCMA bispecific antibody. The ECOG performance status is documented using the scoring method in Table 2.

TABLE 2
Eastern Cooperative Oncology Group (ECOG) Performance Score

| Grade | ECOG |
|---|---|
| 0 | Fully active, able to carry on all pre-disease performance |
| | without restriction. |
| 1 | Restricted in physically strenuous activity but ambulatory |
| | and able to carry out work of a light or sedentary nature, |
| | e.g., light housework, office work. |
| 2 | Ambulatory and capable of all self-care but unable to carry |
| | out any work activities. Up and about more than 50% of waking |
| | hours. |
| 3 | Capable of only limited self-care, confined to bed or chair |
| | more than 50% of waking hours. |
| 4 | Completely disabled. Cannot carry on any self-care. Totally |
| | confined to bed or chair. |

### 5.4.3 Clinical Laboratory Tests

Aspects of the disclosure may include obtaining samples for the clinical laboratory tests outlined in Table 3, at a minimum at Screening (by a central laboratory), and locally at subsequent visits, the EOT Visit, and the 90-day follow-up visit.

A certified laboratory can be utilized to process and provide results for the clinical laboratory tests. Laboratory reference ranges are obtained prior to the initiation of the study. The baseline laboratory test results for clinical assessment for a particular test are defined as the last measurement prior to the initial dose of the CD3×BCMA bispecific antibody.

TABLE 3
Clinical Laboratory Tests

| Laboratory | Parameter | |
|---|---|---|
| Hematology | Hematocrit | Basophils (if detected; |
| | Hemoglobin | absolute count and %) |
| | RBC count | Eosinophils (if detected; |
| | WBC count | absolute count and %) |
| | Neutrophils (absolute | Platelet count (estimate |
| | count and %) | not acceptable) |
| | Bands (if detected) | MCH |
| | Lymphocytes (absolute | MCV |
| | count and %) | MCHC |
| Coagulation | INR | aPTT |
| Panel | PT | |
| Serum | BUN | Serum pregnancy test |
| Chemistry | Creatinine | (where applicable) |
| | Total bilirubin | Total protein |
| | Albumin | Glucose |
| | AST | Chloride |
| | ALT | Bicarbonate/CO2 |
| | Alkaline phosphatase | LDH |
| | Sodium | Magnesium |
| | Potassium | GGT |
| | Calcium | |
| Cytokines^{a} | IL-2 | IFN-gamma |
| | IL-6 | IL-1 |
| | TNF-alpha | IL-10 |
| T-Cell | T-Cell Subsets by Flow | T-Cell Receptor Sequencing |
| Hepatitis B and | HBsAg | HCV Ab |
| C (Screening | | |
| Urine Tests | 24-hour urine collection for: | Protein |
| | Total protein | Glucose |
| | UPEP with M-protein | Blood |
| | quantitation | Urobilinogen |
| | UIFE | Bilirubin |
| | Specific gravity | Microscopic examination |
| | Ketones | |
| | pH | |
| Mveloma- | Bone marrow aspiration/biopsy | SFLC |
| | with: | Quantitative immuno-globulins |
| | Immunohistochemistry and quantitation | (IgG, IgA, IgM; at baseline) |
| | of clonal plasma cell population | Beta-2-microglobulin (at |
| | BCMA receptor density on | baseline) |
| | myeloma cells by | Exploratory: |
| | standardized flow cytometry | [Soluble BCMA] in serum |
| | Cytogenetics and FISH | by ELISA |
| | analysis | [APRIL] in serum |
| | SIFE | |
| | SPEP with M-protein | |
| | quantitation | |

Abbreviations:
Ab = antibody;
APRIL = a proliferation-inducing ligand;
aPTT = activated partial thromboplastin time;
ALT = alanine aminotransferase;
AST = aspartate aminotransferase;
BCMA = B cell maturation antigen;
BUN = Blood urea nitrogen;
ELISA = enzyme linked immunosorbent assay;
FISH = fluorescence in situ hybridization;
GGT = gamma-glutamyltransferase;
HBsAg = hepatitis B surface antigen;
HCV = hepatitis C virus;
IFN = interferon;
IL = interleukin;
LDH = lactate dehydrogenase;
MCH = mean corpuscular hemoglobin;
PT = prothrombin time;
MCHC = mean corpuscular hemoglobin concentration;
MCV = mean corpuscular volume;
RBC = red blood cell;
SFLC = serum free light chains;
SIFE = serum immunofixation electrophoresis;
SPEP = serum protein electrophoresis;
TNF = tumor necrosis factor;
UIFE = urine immunophoresis electrophoresis;
UPEP = urine protein electrophoresis;
WBC = white blood cell;
^{a}The enumerated cytokines will be the minimally evaluated cytokines; analysis of additional cytokines in the samples submitted for cytokine analysis may be performed.

### 5.4.4 Tumor Assessments

Aspects of the disclosure may involve performing a baseline skeletal survey with positron emission tomography (PET)-CT, contrast enhanced CT, or magnetic resonance imaging (MRI) for each patient within 28 days prior to administration of the first maintenance dose of the CD3×BCMA bispecific antibody; CT and/or MRI may also be performed for extramedullary disease assessment if clinically indicated. Imaging is repeated as clinically indicated. The same modality should be used for a subject at each visit where imaging is required, if possible.

### 5.4.5 Tumor Tissue

Aspects of the disclosure may involve collecting, at screening, adequate archival tumor tissue (collected within the past 6 months prior to screening) and/or newly obtained biopsies for each patient; if no archival tumor tissue is available, a pre-treatment bone marrow biopsy is taken in embodiments. An “adequate” archival biopsy is defined as sufficient formalin-fixed paraffin embedded (FFPE) material (either block or slides) to perform and interpret 8 to 10 hematoxylin and eosin and/or immunohistochemical stains on the patient's tumor and accompanied by a flow-cytometric report including plasma cell markers (at least CD38 and CD138). In embodiments, a bone marrow biopsy/aspirate is performed at cycle 3 day 1 (C3D1), in addition to IMWG mandated biopsies/aspirates at suspected CR, and when possible, at suspected progression.

In embodiments, both pre-treatment and progression biopsies are taken from the same lesion, or at least from the same anatomical location, of a patient. Collection of paired newly obtained tumor samples is used to assess the CD3×BCMA bispecific antibody PD in the tumor microenvironment. In embodiments, the bone marrow biopsies are analyzed by flow cytometry to quantitate BCMA density on the patient's tumor cells. In embodiments, studies such as cytogenetics, fluorescence in situ hybridization (FISH), or sequencing studies of tumor cells, and evaluation of T-cell subsets by flow cytometry are also performed. In addition, the tumor mutation load is examined in embodiments.

### 5.4.6 Activity Variables

In embodiments of the disclosure, activity may be measured by changes in SPEP, UPEP, and/or FLC (Kumar S et al., International Myeloma Working Group consensus criteria for response and minimal residual disease assessment in MM, The Lancet Oncology. 2016; 17 (8): e328-46). In embodiments, the same parameter(s) used to meet eligibility criteria for treatment are used to evaluate response. In embodiments, a laboratory value that indicates clinical activity (Table 4) is verified by a second test, which may be conducted as soon as the results from the first test are available. In embodiments, activity endpoints (determined using IMWG uniform response criteria) include objective response rate (ORR, stringent complete response [sCR]+complete response [CR]+very good partial response [VGPR]+partial response [PR]), clinical benefit rate (CBR; defined as CR+PR+MR for 24 weeks), overall survival (OS), progression-free survival (PFS), time to progression (TTP), time to response (TTR) and duration of objective response (DOR).

TABLE 4
Activity Measurements
Response Criteria
IMWG MRD criteria (requires a complete response as defined below)

| | |
|---|---|
| Sustained MRD-negative | MRD negativity in the marrow (NGF or NGS. or both) and by imaging as |
| | defined below, confirmed minimum of 1 year apart. Subsequent evaluations |
| | can be used to further specify the duration of negativity (e.g., MRD- |
| | negative at 5 years) |
| Flow MRD-negative | Absence of phenotypically aberrant clonal plasma cells by NGF-on bone |
| | marrow aspirates using the EuroFlow standard operation procedure for |
| | MRD detection in multiple myeloma (or validated equivalent method) with |
| | a minimum sensitivity of 1 in 10⁵ nucleated cells or higher |
| Sequencing MRD-negative | Absence of clonal plasma cells by NGS on bone marrow aspirate in which |
| | presence of a clone is defined as less than two identical sequencing reads |
| | obtained after DNA sequencing of bone marrow aspirates using the |
| | LymphoSIGHT ™ platform (or validated equivalent method) with a |
| | minimum sensitivity of 1 in 10⁵ nucleated cells or higher |
| Imaging plus MRD- | MRD negativity as defined by NGF or NGS plus disappearance of every |
| negative | area of increased tracer uptake found at baseline or a preceding PET/CT or |
| | decrease to less mediastinal blood pool SUV or decrease to less than that of |
| | surrounding normal tissue |

Standard IMWG Response Criteria

| | |
|---|---|
| Stringent complete | Complete response as defined below plus normal FLC ratio and absence of |
| response | clonal cells in bone marrow biopsy by immunohistochemistry (K/Λ ratio ≤4:1 |
| | or ≥1:2 for K and Λ patients, respectively, after counting ≥100 plasma |
| | cells) |
| Complete response | Negative immunofixation on the serum and urine and disappearance of any |
| | soft tissue plasmacytomas and <5% plasma cells in bone marrow aspirates |
| Very good partial response | Serum and urine M-protein detectable by immunofixation but not on |
| | electrophoresis¹ or ≥90% reduction in serum M-protein plus urine |
| | M-protein level <100 mg per 24 h |
| Partial response | ≥50% reduction of serum M-protein plus reduction in 24 h urinary |
| | M-protein by ≥90% or to <200 mg per 24 h; |
| | If the serum and urine M-protein are unmeasurable, a ≥50% decrease in the |
| | difference between involved and uninvolved FLC levels is required in place |
| | of the M-protein criteria; |
| | If serum and urine M-protein are unmeasurable, and serum-free light assay |
| | is also unmeasurable, ≥50% reduction in plasma cells is required in place |
| | of M-protein, provided baseline bone marrow plasma-cell percentage was ≥30%. |
| | In addition to these criteria, if present at baseline, a ≥50% |
| | reduction in the size (SPD)¹ of soft tissue plasmacytomas is also required |
| Minimal response | ≥25% but ≤49% reduction of serum M-protein and reduction in 24-h urine |
| | M-protein by 50-89%. In addition to the above listed criteria, if present at |
| | baseline, a ≥50% reduction in the size (SPD of soft tissue plasmacytomas |
| | is also required |
| Stable disease | Not recommended for use as an indicator of response; stability of disease |
| | is best described by providing the time-to-progression estimates. Not |
| | meeting criteria for complete response, very good partial response, partial |
| | response, minimal response, or progressive disease |
| Progressive disease | Any one or more of the following criteria: |
| | Increase of 25% from lowest confirmed response value in one or more of |
| | the following criteria: |
| | Serum M-protein (absolute increase must be ≥0 5 g/dL); |
| | Serum M-protein increase ≥1 g/dL, if the lowest M component was ≥5 |
| | g/dL; |
| | Urine M-protein (absolute increase must be ≥200 mg 24 h); |
| | In patients without measurable serum and urine M-protein levels, the |
| | difference between involved and uninvolved FLC levels (absolute increase |
| | must be ≥10 mg dL); |
| | In patients without measurable serum and urine M-protein levels and |
| | without measurable involved FLC levels, bone marrow plasma-cell |
| | percentage irrespective of baseline status (absolute increase must |
| | be ≥10%); |
| | Appearance of a new lesion(s), ≥50% increase from nadir in SPI¹ ≥ of ≥1 |
| | lesion, or ≥50% increase in the longest diameter of a previous lesion ≥1 |
| | cm in short axis, ≥50% increase in circulating plasma cells (minimum of |
| | 200 cells per μL) if this is the only measure of disease |
| Clinical relapse | Clinical relapse requires one or more of the following criteria: |
| | Direct indicators of increasing disease and/or end organ dysfunction |
| | (CRAB features) related to the underlying clonal plasma-cell proliferative |
| | disorder. It is not used in calculation of time to progression or progression- |
| | free survival but is listed as something that can be reported optionally or for |
| | use in clinical practice; |
| | Development of new soft tissue plasmacytomas or bone lesions |
| | (osteoporotic fractures do not constitute progression); |
| | Definite increase in the size of existing plasmacytomas or bone lesions. A |
| | definite increase is defined as a 50% (and ≥1 cm) increase as measured |
| | serially by the SPD of the measurable lesion. |
| | Hypercalcemia (≥11 mg/L); |
| | Decrease in hemoglobin of ≥2 g/dL not related to therapy or other non- |
| | myeloma-related conditions; |
| | Rise in serum creatinine by 2 mg/dL or more from the start of the therapy |
| | and attributable to myeloma; |
| | Hyperviscosity related to serum paraprotein |
| Relapse from complete | Any one or more of the following criteria: |
| response (to be used only if | Reappearance of serum or urine M-protein by immunofixation or |
| the end point is disease- | electrophoresis. |
| free survival) | Development of ≥5% plasma cells in the bone marrow; |
| | Appearance of any other sign of progression (i.e., new plasmacytoma, |
| | lytic bone lesion, or hypercalcemia see above) |
| Relapse from MRD | Any one or more of the following criteria: |
| negative (to be used only if | Loss of MRD negative state (evidence of clonal plasma cells on NGF or |
| the end point is disease- | NGS, or positive imaging study for recurrence of myeloma); |
| free survival) | Reappearance of serum or urine M-protein by immunofixation or |
| | electrophoresis. |
| | Development of ≥5% clonal plasma cells in the bone marrow; |
| | Appearance of any other sign of progression (i.e., new plasmacytoma, |
| | lytic bone lesion, or hypercalcemia) |

Abbreviations: ASCT = autologous stem cell transplantation; CRAB features = calcium elevation, renal failure, anemia, lytic bone lesions; ¹⁸F-FDG PET = ¹⁸F-fluorodeoxyglucose PET; FCM = flow cytometry; FLC = free light chain; IMWG = International Myeloma Working Group, MFC = multiparameter flow cytometry; M-protein—myeloma protein; MRD = minimal residual disease; NGF = next-generation flow; NGS—next-generation sequencing; SPD = sum of the products of the maximal perpendicular diameters of measured lesions; SUVmax = maximum standardized uptake value.

### 5.4.7 Dose Limiting Toxicity (DLT)

In embodiments of the disclosure, the DLT observation period is the first 21 days after the first dose of the CD3×BCMA bispecific antibody; dose limiting toxicities are determined on events that occur during the first observation period. A drug related event is defined as any adverse reaction that cannot be definitively attributed to the patient's underlying disease, other medical condition, or a concomitant medication or procedure by the investigator or Medical Monitor. The NCI-CTCAE version 5.0 will be used. DLT definitions are provided herein.

### Non-hematologic Dose-limiting Toxicity

A non-hematologic DLT is defined as any of the following TEAEs:
Non-hematological AEs Grade ≥3 with the following exceptions:
   Grade 3 or 4 isolated electrolyte abnormalities (i.e., those occurring without clinical consequence) that resolve, with or without intervention, to Grade <2 within 72 hours.
   Grade 3 hypo/hyperglycemia responsive to optimal medical management within 72 hours.
   Grade 3 nausea/vomiting/diarrhea responsive to optimal medical management within 72 hours.
   Alopecia or vitiligo of any grade.
   Grade 3 fatigue lasting <10 days.
Any AE that requires a delay in initiation of the next scheduled cycle by >21 days.

### Hematologic Dose-limiting Toxicity

A hematologic DLT is defined as any of the following:
≥ Grade 3 CRS.
Grade 4 neutropenia for >5 days.
Grade 3 thrombocytopenia requiring platelet transfusion or Grade 4 thrombocytopenia.
   During the course of the study, subjects with platelet counts <10,000/mm³ should be monitored at least every 72 hours (or more often at investigator's discretion) until recovery to >50,000/mm³. Platelet transfusions should be administered per Principal Investigator discretion and according to institutional guidelines. Recovery to >50,000/mm³ is required to continue treatment.
Grade 3 anemia associated with clinically significant symptoms of hypoxia requiring red blood cell transfusion or Grade 4 anemia, unrelated to underlying disease.
   Red cell transfusion in the absence of clinically significant hypoxic symptoms (i.e., tachypnea, decreased oxygen saturation, etc.) will not be considered a DLT. Moderate anemia is not an exclusion criterion for subjects entering the study and is an anticipated finding in subjects with MM. Nevertheless, subjects with hemoglobin <8 g/dL should be monitored at least every 72 hours (or more often at investigator's discretion) until recovery to >8 g/dL. Red cell transfusions should be administered according to institutional guidelines. Recovery to ≥8 g/dL is required to continue treatment.
Lymphopenia will not be considered a DLT.
Any AE that requires a delay in initiation of the next scheduled cycle by >21 days.

### CRS Dose-limiting Toxicity

Aspects of the disclosure involve monitoring for evidence of CRS in patients. CRS, with or without the presence of neurotoxicity, is the primary toxicity associated with T-cell redirection therapy (CARs and T-BsAbs/BiTEs). CRS occurs due to hyper-activation of the immune system and is mediated predominantly by the secretion of pro-inflammatory cytokines (most importantly IL-6). Signs and symptoms are those of systemic inflammation, like sepsis, anaphylaxis and tumor lysis syndrome, and include the following: high fever/rigors, hypotension, hypoxia, neurologic changes, pain, nausea, and headache. Meta-analyses show that clinical findings, specifically fever, are usually the first indicators of CRS onset (Hay K A et al., Blood, January 1:2017; Wang Z et al., Biomarker Research. 2018; 6 (1): 4.). CRS historically occurs within 14 days of first CAR/T-BsAb administration and does not usually occur in subsequent cycles. If CRS symptoms are suspected, Table 5 may be used to grade the toxicity.

TABLE 5
Guidelines for CRS Grading

| | CRS Grade 1 | CRS Grade 2 | CRS Grade 3 | CRS Grade 4 |
|---|---|---|---|---|
| Temperature > | Yes | Any | Any | Any |
| 38.5 C. | | | | |
| SBP ≤ 90 | No | Responsive to IV | Requiring one | Life-threatening |
| mmHg | | fluids | pressor | |
| Supplement | No | FiO2 < 40% | FiO2 ≥ 40% | Ventilator |
| O2 for SO2 | | | | |
| Organ toxicity | Grade 1 | Grade 2 | Grade 3 | Grade 4 |
| | | | (with increased | |
| | | | AST / ALT | |
| | | | Grade 4) | |

Source: CTCAE v 5.0

### Neurological Dose-limiting Toxicity

Aspects of the disclosure involve monitoring for evidence of neurological toxicity (NT). Neurological toxicity (NT) arises from unclear etiology but has been postulated to stem from endothelial activation/microangiopathy, possibly downstream of IL-1 secretion by monocytes/macrophages (Gust J et al., Cancer Discovery, 2017; 7 (12): 1404-1419; Giavridis T et al., Nature Medicine, 2018 24 (6): 731-738.; Norelli M et al., Nature Medicine, 2018; 24 (6): 739-748.). Onset usually occurs with or after CRS (mostly CRS Grade ≥3). Isolated NT has been described after administration of anti-CD19 T-BsAbs (Velasquez M P et al., Blood. 2017; 131 (1): 30-38.). Symptoms of NT include delirium, headache, agitation, aphasia, CNS bleed, ataxia, confusion, seizure, somnolence, and tremor. Suggested guidelines for management of NTs are provided in Table 6.

TABLE 6
Suggested Guidelines for Management of Neurological Toxicity

| AE Category | Guidelines |
|---|---|
| Neurologic AE after | Consult neurologist |
| dosing with etentamig | Continue neurologic monitoring |
| | Prophylactic anti-epileptic (i.e., levetiracetam) |
| | Consider MRI/CT, EEG, and/or LP |
| | Contact Medical Monitor |
| If the AE is Grade 2+, add | Consider Corticosteroids (e.g., dexamethasone 10 to |
| | 20 mg IV every 12 to 24 hours, higher doses or with |
| | more frequency, if clinically indicated) |
| If the AE is Grade 3+, add | High-dose corticosteroids (methylprednisolone 2 mg/kg |
| | loading dose followed by 2 mg/kg/day divided 4 times |
| | Higher/more frequent doses if indicated. Taper when |
| | Grade 1 or better) |
| | Anti-epileptics for seizures/seizure-like activity |
| | ICU monitoring |
| | Airway protection |
| | Consider continuous EEG monitoring |
| If the AE is Grade 4+, add | If cerebral edema/other Grade 4 toxicity is rapid |
| | onset or the subjects is unresponsive to above: |
| | 1 g/day × 3 days, followed if clinically indicated |
| | by taper. |
| | Consider hyperventilation/hyperosmolar therapy for |
| | cerebral edema. |

Abbreviations: AE = adverse event; CT = computed tomography; EEG = electroencephalogram; ICU = intensive care unit; IV = intravenous; LP = lumbar puncture; MRI = magnetic resonance imaging.

### 5.4.8 Activity Analysis

Aspects of the disclosure may involve evaluating response and disease progression using IMWG uniform response criteria. In embodiments, ORR, DOR, PFS and CBR are determined. In embodiments, Kaplan-Meier estimates for PFS and associated CI of the median PFS, OS, and TTP are determined.

of this study.

### 5.4.9 Objective Response Rate (ORR)

Aspects of the disclosure may involve determining an object response rate (ORR). Objective response rate is defined as the proportion of subjects with a confirmed partial or complete response to treatment. In embodiments, the ORR for each dose cohort is estimated with all testing sites pooled. In embodiments, the 2-sided 80% exact binomial CIs of ORR are also summarized using the Clopper-Pearson method along with the best overall response (CR, PR, SD, PD).

### 5.4.10 Progression-Free Survival (PFS)

Aspects of the disclosure may involve determining progression-free survival (PFS). Progression-free survival time is defined as the time from the first maintenance dose of the CD3×BCMA bispecific antibody to progression or death, whichever occurs first. In embodiments, a patient is censored at the date of last tumor assessment if neither event occurs. In some embodiments, the Kaplan-Meier method will be used to analyze PFS.

### 5.4.11 Duration of Objective Response (DOR)

Aspects of the disclosure may involve determining a duration of objective response (DOR). The duration of objective response for a subject is defined as the time from the initial objective response to disease progression or death, whichever occurs first. In embodiments, if a subject does not progress or die then the subject will be censored at the date of the last tumor assessment, similar to the censoring rules for the PFS analysis. In embodiments, the DOR is analyzed in the same fashion as for the PFS analysis.

### 5.4.12 Clinical Benefit Rate (CBR)

Aspects of the disclosure may involve determining a clinical benefit rate (CBR). Clinical benefit rate is defined as the proportion of subjects with a confirmed complete, partial or minimal response for at least 24 weeks after responding to treatment. In embodiments, the CBR for each arm is estimated with all testing sites pooled. In embodiments, the 2-sided 80% exact binomial CIs of the CBR will also be summarized using the Clopper-Pearson method.

### 5.4.13 Safety Analysis

Aspects of the disclosure may involve conducting one or more safety analyses. In embodiments, at the end of a course of treatment, the safety of the CD3×BCMA bispecific antibody is assessed by evaluating the AEs, SUSARs/SAEs, changes in laboratory determinations, vital sign parameters and all other available relevant data. In embodiments, the methods involve providing descriptive statistics for the continuous variables and the frequencies /percentages for the discrete variables. In embodiments, the safety population allows for detection of SAEs occurring in as little as 21% of subjects with 80% confidence.

### 5.4.14 Adverse Events

Aspects of the disclosure may involve analyzing adverse events, including, but not limited to, treatment emergent adverse events (TEAEs). A treatment emergent adverse event (TEAE) is defined as an event that occurs or worsens on or after the first maintenance dose of the CD3×BCMA bispecific antibody until 90 days following discontinuation of drug administration have elapsed, or until subjects start another anticancer therapy, whichever occurs earlier.

In embodiments, TEAEs are summarized by dose cohort and overall including drug-related AEs, AEs by intensity, deaths, SAEs, and discontinuations due to AEs. In some embodiments, DLTs for the dose cohorts in the Monotherapy Dose Escalation Phase are summarized similarly by cohort and overall. In embodiments, additional summaries and/or listings for AEs of special interest are also provided.

### 5.4.15 Clinical Laboratory Tests

Aspects of the disclosure may involve performing baseline laboratory tests for patients receiving treatment. In embodiments, disease response assessment laboratory tests from subsequent time points are also performed. In embodiments, changes from baseline in clinical laboratory results are analyzed and summarized by dose cohort and time point using descriptive statistics. In embodiments, summaries of shifts from baseline to last available visit are provided. In embodiments, shifts are calculated as the proportion of subjects at baseline with values that are below, within, or above the normal range for a particular lab test, relative to the proportion of subjects at the Final Visit with values that are below, within, or above the normal range. In embodiments, lab abnormalities and treatment-emergent lab abnormalities meeting the NCI-CTCAE version 5.0 are summarized by treatment arm and overall.

### 5.4.16 Key Eligibility Criteria

### Tabulations and Summary Statistics

In embodiments, serum concentrations of the CD3×BCMA bispecific antibody and PK parameter values may be tabulated for each subject and each dose level, and summary statistics are computed for each sampling time and each parameter.

### Dose Proportionality Analysis:

In embodiments, pharmacokinetic parameters of the CD3×BCMA bispecific antibody from a particular dosing regimen may be assessed on Cycle 1 Day 1 are analyzed as follows. An analysis is performed for dose-normalized Cmax and dose-normalized AUC. The model used for the statistical analyses includes the dose level of the CD3×BCMA bispecific antibody as a categorical variable. In embodiments, covariates such as age, ethnicity, gender, and others that might explain some of the variability in the population are included in an initial model. In embodiments, a covariate may be dropped from the model if the regression coefficient is not significant at alpha level 0.10. In embodiments, a natural logarithmic transformation is employed for Cmax and the AUCs unless the data clearly indicate that other transformation or the untransformed variable provides more nearly symmetric probability distributions and/or more nearly homogenous variances across dose levels. When at least three dose levels of the CD3×BCMA bispecific antibody are studied, a test is performed on a contrast in the dose level effects chosen to be sensitive to an approximately linear function of dose or the logarithm of dose.

### Missing Values and Model Violations

In embodiments, all available data can be included in any dose proportionality analyses. In embodiments, one or more data points may be excluded from an analysis, provided an appropriate justification is present. Normally, values of PK variables (Cmax, AUC, etc.) are determined without replacing missing individual concentration values, by simply using the available data. However, if a missing individual concentration results in a PK parameter value that may be too low or too high to a meaningful degree, the value of the PK parameter may tentatively be considered missing. In such cases, a value for the missing individual concentration may be imputed so that an appropriate value of the PK parameter can be included in an analysis.

In embodiments, the imputed value is obtained using appropriate methodology that considers the individual characteristics of the subject.

If an outlier is identified and/or a pronounced non-normal probability distribution is observed (after logarithmic transformation for Cmax and AUC) then a non-parametric analysis may also be performed. Such a model violation may be identified by graphical methods, measures of non-normality (e.g., skewness, kurtosis) or other appropriate methods. If different dose levels have unequal variances to the extent that conclusions might be affected, then approximate methods that allow for unequal variances can be used. In embodiments, the possibility of bias from missing data of subjects who prematurely discontinued treatment due to an adverse event can be addressed.

### 6. EXAMPLES

The examples in this section are offered by way of illustration, and not by way of limitation. The following examples are presented as exemplary embodiments of the disclosure.

### 6.1 EXAMPLE 1-Administration Of Etentamig 60 mg Q4W Is Clinically Efficacious

Etentamig is a CD3×BCMA bispecific antibody incorporating a unique anti-CD3 moiety that preferentially activates effector over regulatory T-cells and uncouples cytokine release from anti-tumor activity, as well as 2 heavy-chain-only anti-BCMA moieties for a 2:1 TAA to CD3 stoichiometry.

This Example demonstrates that etentamig 60 mg every four weeks (Q4W) is efficacious. Efficacy was evaluated in 32 subjects treated with 20 mg of etentamig Q3W, 55 subjects treated with 40 mg of etentamig Q3W, 60 subjects treated with 60 mg of etentamig Q3W, and 21 subjects treated in the 60 mg Q4W.

Patients receiving the 20 mg, 40 mg and 60 mg Q3W doses were pretreated with H2 antagonist, acetaminophen and antihistamine or equivalents, and dexamethasone 10 mg IV or equivalent 15 to 60 minutes before etentamig infusion; after the first cycle, dexamethasone could be tapered if no CRS event occurred in a prior cycle. Patients receiving the 60 mg Q4W dose were pretreated with acetaminophen (every 6 hours for first 24 hours), diphenhydramine and famotidine or equivalents orally/IV 15 to 60 minutes prior to etentamig infusion, plus dexamethasone 8 mg orally 12 hours prior to infusion, 8 mg orally 3-4 hours prior to infusion, and 20 mg IV 15 to 60 minutes prior to infusion (total 36 mg dexamethasone); after the first cycle, dexamethasone could be tapered if no CRS event occurred in a prior cycle.

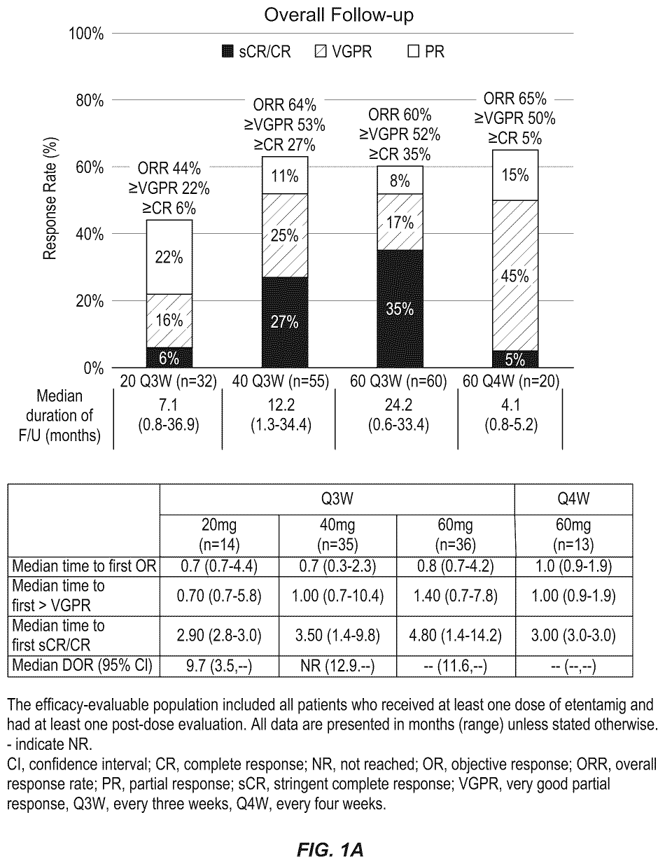

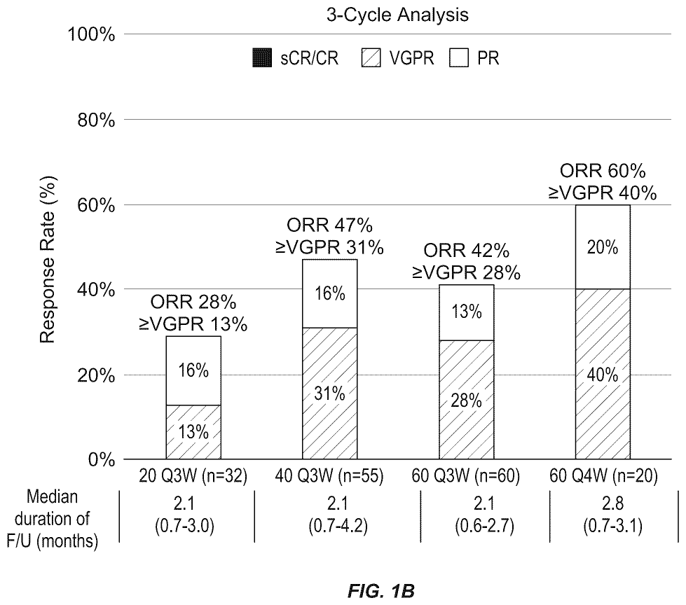

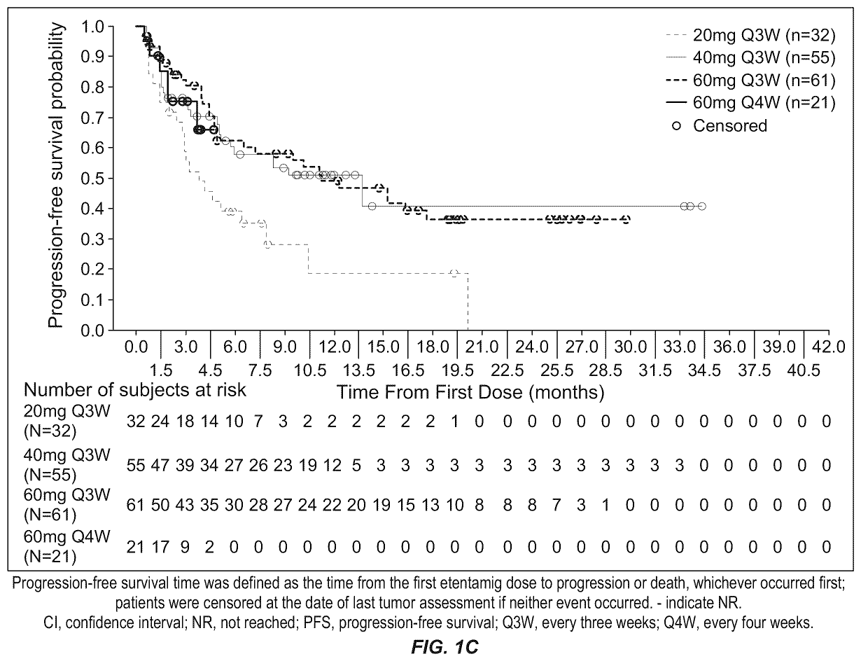

In the overall efficacy-evaluable population, of 218 subjects with RRMM with median of 5 prior lines of therapy, including 80% with triple-class refractory disease, the ORR, ≥ VGPR, and sCR/CR rates were 56%, 44%, and 24%, respectively (Table 7, FIG. 1A). At the 20-mg Q3W dose level (n=32), the ORR, >VGPR, and sCR/CR rates were 44%, 22%, and 6%, respectively. In subjects receiving etentamig at 40 mg Q3W and above, consistent and robust efficacy of etentamig monotherapy was observed as indicated by an ORR of 64% in the 40 mg Q3W, 60% in the 60 mg Q3W, and 65% in the 60 mg Q4W cohorts. Additionally, a similar trend of superior ≥VGPR rates is noted at and above dose level 40 mg Q3W (53% at 40 mg Q3W, 52% at 60 mg Q3W, and 50% at 60 mg Q4W) compared with those at 20 mg Q3W (22%). The observed ORR and >VGPR rates are similar for the 40 mg Q3W, 60 mg Q3W, and 60 mg Q4W dose levels.

TABLE 7
Summary of Response in Efficacy-evaluable
Population for Overall Follow Up

| | 20 Q3W | 40 Q3W | 60 Q3W | 60 Q4W |
|---|---|---|---|---|
| | n (%) | n (%) | n (%) | n (%) |
| Median | 7.1 | 12.2 | 24.2 | 4.1 |
| duration | (0.8-36.9) | (1.3-34.4) | (0.6-33.4) | (0.8-5.2) |
| of follow-up | | | | |
| (months) | | | | |
| ORR | 14 (44) | 35 (64) | 36 (60) | 13 (65) |
| (95% CI) | (26.4-62.3) | (49.6-76.2) | (46.2-72.4) | (40.8-84.6) |
| ≥VGPR | 7 (22) | 29 (53) | 31 (52) | 10 (50) |
| (95% CI) | (9.3-40.0) | (38.8-66.3) | (38.4-64.8) | (27.2-72.8) |
| ≥CR | 2 (6) | 15 (27) | 21 (35) | 1 (5) |
| (95% CI) | (0.8-20.8) | (16.1-41.0) | (23.1-48.4) | (0.1-24.9) |

Due to sequential enrollment, the median duration of follow-up varies across the expansion cohorts. To balance and equalize the comparison of the efficacy profile in these cohorts, the ORR, ≥VGPR, and sCR/CR rates were evaluated using adjusted 3-cycle follow-up for each subject (Table 8, FIG. 1B). At the 20-mg Q3W dose level (n=32), the ORR, ≥ VGPR, and sCR/CR rates were 28%, 13%, and 0%, respectively. At the 40-mg Q3W dose level (n=55), the ORR, ≥VGPR, and sCR/CR rates were 47%, 31%, and 0%, respectively. At the 60-mg Q3W dose level (n=60), the ORR, >VGPR, and sCR/CR rates were 42%, 28%, and 0%, respectively. At the 60-mg Q4W dose level (n=20), the ORR, >VGPR, and sCR/CR rates were 60%, 40%, and 0%, respectively.

TABLE 8
Summary of Response in Efficacy-evaluable
Population for 3-Cycle Analysis

| | 20 Q3W | 40 Q3W | 60 Q3W | 60 Q4W |
|---|---|---|---|---|
| | n (%) | n (%) | n (%) | n (%) |
| Median | 2.1 | 2.1 | 2.1 | 2.8 |
| duration | (0.7-3.0) | (0.7-4.2) | (0.6-2.7) | (0.7-3.1) |
| of follow-up | | | | |
| (months) | | | | |
| ORR | 9 (28) | 26 (47) | 25 (42) | 12 (60) |
| (95% CI) | (13.7-46.7) | (33.7-61.2) | (29.1-55.1) | (36.1-80.9) |
| ≥VGPR | 4 (13) | 17 (31) | 17 (28) | 8 (40) |
| (95% CI) | (3.5-29.0) | (19.1-44.8) | (17.5-41.4) | (19.1-63.9) |
| ≥CR | 0 (0) | 0 (0) | 0 (0) | 0 (0) |
| (95% CI) | (0-13.7) | (0-6.5) | (0-6.0) | (0-1.7) |

Sixteen patients had MRD-negative results (20 mg Q3W: 1 [3%]; 40 mg Q3W: 7 [13%]; 60 mg Q3W: 9 [15%]; 60 mg Q4W: 0), as measured by centralized testing of bone marrow aspirate by next generation sequencing.

In addition, Median PFS (95 CI) in patients receiving Q3W doses was 3.8 months (2.4, 7.9) for 20 mg, 13.7 (5.0,--) for 40 mg, 11.2 (4.8,--) for 60 mg, and not reached (3.7,--) in patients treated with 60 mg Q4W (FIG. 1C).

Etentamig at 20 mg Q3W, 40 mg Q3W, 60 mg Q3W, and 60 mg Q4W led to rapid and transient production of key proinflammatory cytokines (IL-6, IL-8, IFN-γ, and TNF-α), and reduction of soluble BCMA levels over time that were associated with disease response. Etentamig also promoted T-cell activation and proliferation with upregulation of CD69 and KI67 markers on peripheral CD4 and CD8 T cells in evaluable patients.

Taken together the clinical data indicate that dosing at the Q4W interval with 60 mg can maintain robust efficacy.

### 6.2 EXAMPLE 2-Etentamig 60 mg Q4W Dose Is Safe And Tolerable With Low Incidence Of CRS

This Example demonstrates that etentamig 60 mg every four weeks (Q4W) can maintain robust efficacy, with the potential for an improved safety profile.

Briefly, a total of 220 subjects were treated with etentamig (Table 9). The median age for all patients was 68 years (35-92), with a triple-class refractory population of 80% (Table 9).

TABLE 9
Baseline Characteristics

| | | | | | |
|---|---|---|---|---|---|
| Subject | | | | | |
| Demographics, | | | | | |
| Disposition, and | | | | | |
| Disease | 20 mg Q3W | 40 mg Q3W | 60 mg Q3W | 60 mg Q4W | All Subjects^{a} |
| Characteristics | (N = 32) | (N = 55) | (N = 61) | (N = 21) | (N = 220)^{b} |

Demographics

| | | | | | |
|---|---|---|---|---|---|
| Age, median | 68 | 68 | 68 | 67 | 68.0 |
| (range), | (36-89) | (42-84) | (35-92) | (51-87) | (35-92) |
| years | | | | | |

Sex, n (%)

| | | | | | |
|---|---|---|---|---|---|
| Female | 16 | 19 | 29 | 11 | 99 |
| | (50) | (35) | (48) | (52) | (45) |
| Male | 16 | 36 | 32 | 10 | 121 |
| | (50) | (65) | (52) | (48) | (55) |

Race, n (%)

| | | | | | |
|---|---|---|---|---|---|
| White | 25 | 40 | 47 | 16 | 171 |
| | (78) | (73) | (77) | (76) | (78) |
| Black or African | 6 | 11 | 10 | 3 | 37 |
| American | (19) | (20) | (16) | (14) | (17) |
| Asian | 1 | 1 | 1 | 0 | 3 |
| | (3) | (2) | (2) | | (1) |
| Not reported | 0 | 3 | 3 | 2 | 9 |
| | | (5) | (5) | (10) | (4) |

Disease Characteristics
Baseline ECOG Performance Status, n (%)

| | | | | | |
|---|---|---|---|---|---|
| 0 | 5 | 14 | 21 | 3 | 58 |
| | (16) | (25) | (34) | (14) | (26) |
| 1 | 24 | 33 | 37 | 12 | 134 |
| | (75) | (60) | (61) | (57) | (61) |
| 2 | 3 | 7 | 2 (3) | 6 | 25 |
| | (9) | (13) | | (29) | (11) |

R-ISS disease stage (at screening)

| | | | | | |
|---|---|---|---|---|---|
| I | 12 | 11 | 15 | 7 | 63 |
| | (41) | (20) | (25) | (33) | (29) |
| II | 9 | 29 | 28 | 10 | 96 |
| | (28) | (53) | (46) | (48) | (44) |
| III | 10 | 14 | 17 | 4 | 59 |
| | (31) | (25) | (28) | (19) | (27) |
| No. of prior LoT | 5 | 4 | 4 | 4 | 5.0 |
| (median [range]) | (3-10) | (3-11) | (3-12) | (3-23) | (3-23) |

Extramedullary Plasmocytoma

| | | | | | |
|---|---|---|---|---|---|
| Presence | 7 | 15 | 15 | 4 | — |
| | (22) | (27) | (25) | (19) | |
| Absence | 2 | 39 | 29 | 17 | — |
| | (78) | (71) | (48) | (81) | |

Prior Cancer Therapy, n (%)

| | | | | | |
|---|---|---|---|---|---|
| Triple-class exposed | 32 | 55 | 61 | 21 | 220 |
| | (100) | (100) | (100) | (100) | (100) |
| Triple-class | 24 | 42 | 50 | 17 | 175 |
| refractory^{a} | (75) | (76) | (82) | (81) | (80) |
| Penta-drug exposed | 27 | 48 | 56 | 20 | 198 |
| | (84) | (87) | (92) | (95) | (90) |
| Penta-drug | 8 | 18 | 25 | 8 | 74 |
| refractory^{b} | (25) | (33) | (41) | (38) | (34) |

^{a}Refractory to at least one PI, IMiD, and anti-CD38 mAb (Daratumumab or Isatuximab).
^{b}Refractory to at least two PIs, two IMiDs, and one anti-CD38 mAb (Daratumumab or Isatuximab). All data are n (%) unless stated otherwise.
ECOG, Eastern Cooperative Oncology Group; R-ISS, Revised International Staging System; Q3W, every three weeks; Q4W, every four weeks.

Of the 220 subjects treated with etentamig, 216 (98%) experienced a treatment-emergent adverse event (TEAE) during the study (Table 10). The most common any-grade TEAEs (reported in ≥25% of all subjects) were CRS (57%), anemia (42%), fatigue (38%), diarrhea (32%), neutrophil count decreased (29%), nausea (27%), and lymphocyte count decreased (26%).

TABLE 10
Overview of TEAEs and All Deaths

| | | | | | |
|---|---|---|---|---|---|
| | 20 mg | 40 mg | 60 mg | 60 mg | All |
| | Q3W | Q3W | Q3W | Q4W | Subjects |
| AE Category | (N = 32) | (N = 55) | (N = 61) | (N = 21) | (N = 220) |

Subjects with any, n (%):

| | | | | | |
|---|---|---|---|---|---|
| TEAE (any grade) | 31 (97) | 55 (100) | 61 (100) | 19 (90) | 216 (98) |
| TEAE (grade ≥3) | 30 (94) | 51 (93) | 56 (92) | 14 (67) | — |
| TEAE related to study drug | 28 (88) | 51 (93) | 56 (92) | 19 (90) | 192 (87) |
| (per investigator | | | | | |
| assessment) | | | | | |
| Grade ≥3 TEAE | 25 (78) | 46 (84) | 50 (82) | 12 (57) | 171 (78) |
| SAE | 20 (63) | 32 (58) | 43 (70) | 12 (57) | 137 (62) |
| Related SAE | 8 (25) | 22 (40) | 27 (44) | 9 (43) | 73 (33) |
| TEAE leading to study | 2 (6) | 6 (11) | 6 (10) | 2 (10) | 24 (11) |
| drug discontinuation | | | | | |
| TEAE leading to study | 15 (47) | 28 (51) | 29 (48) | 3 (14) | 94 (43) |
| drug interruption, hold, or | | | | | |
| delay | | | | | |
| TEAE leading to study | 0 | 2 (4) | 4 (7) | 1 (5) | 9 (4) |
| drug dose reduction | | | | | |
| Death | 11 (34) | 11 (20) | 18 (30) | 2 (10) | 68 (31) |
| TEAE leading to death | 6 (19) | 5 (9) | 8 (13) | 2 (10) | 32 (15) |
| TEAE related to study drug | 0 | 2 (4) | 0 | 0 | 2 (1) |
| leading to death | | | | | |
| Death occurring ≤30 days | 5 (16) | 1 (2) | 4 (7) | 2 (10) | 13 (6) |
| after last dose of study drug | | | | | |
| Death occurring >30 days | 6 (19) | 10 (18) | 14 (23) | 0 | 55 (25) |
| after last dose of study drug | | | | | |
| Death occurring ≤90 days | 8 (25) | 5 (9) | 9 (15) | 2 (10) | 38 (17) |
| after last dose of study drug | | | | | |
| Death occurring >90 days | 3 (9) | 6 (11) | 9 (15) | 0 | 30 (14) |
| after last dose of study drug | | | | | |

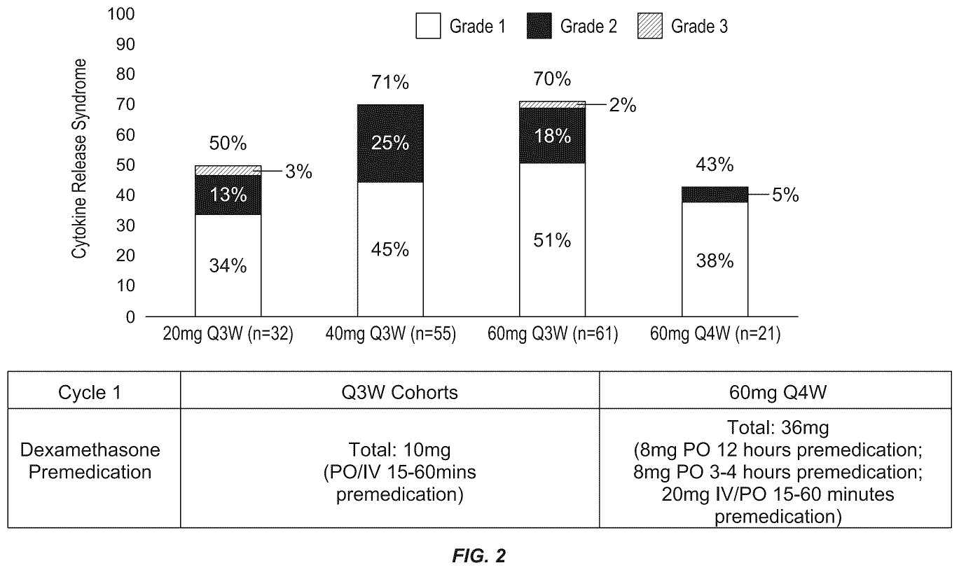

The most frequently reported TEAE was CRS, which was observed at 50%, 71%, 70%, and 43% at 20 mg Q3W, 40 mg Q3W, 60 mg Q3W, and 60 mg Q4W, respectively (FIG. 2, Table 12). Similar incidence of neurotoxicity (including ICANS), infections, and cytopenia was observed for 60 mg Q4W and 40 mg Q3W, compared to 60 mg Q3W. Importantly, lower incidence (43%) and severity (38% Grade 1, 5% Grade 2, 0% Grade ≥3) of CRS was reported at 60 mg Q4W, which included implementation of a modified dexamethasone premedication dose regimen.

TABLE 11
Overview of Additional Hematologic and Nonhematologic Responses

| | | | |
|---|---|---|---|
| 20 mg Q3W | 40 mg Q3W | 60 mg Q3W | 60 mg Q4W |
| (N = 32) | (N = 55) | (N = 61) | (N = 21) |

Hematologic

| | | | | |
|---|---|---|---|---|
| Anemia (any grade) | 16 (50) | 30 (55) | 23 (38) | 10 (48) |
| Anemia (grade ≥3) | 8 (25) | 17 (31) | 8 (13) | 2 (10) |
| Thrombocytopenia (any grade) | 2 (6) | 8 (15) | 9 (15) | 2 (10) |
| Thrombocytopenia (grade ≥3 | 2 (6) | 4 (7) | 5 (8) | 2 (10) |
| Neutropenia (any grade) | 2 (6) | 7(13) | 12 (20) | 1 (5) |
| Neutropenia (grade ≥3) | 1 (3) | 6 (11) | 12 (20) | 1 (5) |
| Lymphopenia (any grade) | 0 (0) | 4 (7) | 2 (3) | 2 (10) |
| Lymphopenia (grade ≥3) | 0 (0) | 3 (5) | 2 (3) | 2 (10) |

Nonhematologic

| | | | | |
|---|---|---|---|---|
| Diarrhea (any grade) | 10 (31) | 17 (31) | 23 (38) | 6 (29) |
| Diarrhea (grade ≥3) | 3 (9) | 0 (0) | 1 (2) | 0 (0) |
| Nausea (any grade) | 6 (19) | 13 (24) | 23 (38) | 3 (14) |
| Nausea (grade ≥3) | 0 (0) | 1 (2) | 0 (0) | 0 (0) |
| Vomiting (any grade) | 4 (13) | 9 (16) | 18 (30) | 0 (0) |
| Vomiting (grade ≥3) | 0 (0) | 1 (2) | 1 (2) | 0 (0) |
| Fatigue (any grade) | 14 (44) | 23 (42) | 23 (38) | 6 (29) |
| Fatigue (grade >3) | 0 (0) | 0 (0) | 0 (0) | 0 (0) |
| Pyrexia (any grade) | 6 (19) | 13 (24) | 15 (25) | 2 (10) |
| Pyrexia (grade ≥3) | 0 (0) | 0 (0) | 1 (2) | 0 (0) |

Grade ≥3 infection TEAEs occurred in 66 (30%) patients. Most common grade 3/4 infections were pneumonia (12%), sepsis (5%), COVID-19 (3%), and urinary tract infection (3%). Grade 3/4 infection occurred in 22%, 24%, and 34% of patients receiving etentamig 20 mg, 40 mg, and 60 mg Q3W dose, respectively, and 10% of those treated with 60 mg Q4W. Most common grade 3/4 infection was pneumonia (12%; Q3W 20 mg 9%, 40 mg 16%; 60 mg 18%; Q4W 60 mg 5%) and sepsis (5%; Q3W 40 mg 4%; Q3W 60 mg 8%)

Importantly, lower incidence (43%) and severity (38% Grade 1, 5% Grade 2, 0% Grade ≥3) of CRS was reported at 60 mg Q4W, which included implementation of a modified dexamethasone premedication dose regimen (FIG. 2, Table 12).

TABLE 12
Overview of CRS

| | 20 mg Q3W | 40 mg Q3W | 60 mg Q3W | 60 mg Q4W |
|---|---|---|---|---|
| CRS | (N = 32) | (N = 55) | (N = 61) | (N = 21) |
| Any grade, n (%) | 16 (50) | 39 (71) | 43 (70) | 9 (43) |
| Grade 1 | 11 (34) | 25 (45) | 31 (51) | 8 (38) |
| Grade 2 | 4 (13) | 14 (25) | 11 (18) | 1 (5) |
| Grade 3 | 1 (3) | 0 (0) | 1 (2) | 0 (0) |
| Serious AE | 5 (16) | 11 (20) | 16 (26) | 6 (29) |
| Median time to onset, | 1.0 (1.0-1.0) | 1.0 (1.0-16.0) | 1.0 (1.0-2.0) | 1.0 (1.0-1.0) |
| days (range) | | | | |
| Median time to | 1.5 (1.0-9.0) | 1.0 (1.0-16.0) | 1.5 (1.0-10.0) | 2.0 (1.0-18.0) |
| resolution, days | | | | |
| (range) | | | | |
| Recurrent in ≥2 | 0(0) | 2(4) | 1(2) | 0(0) |
| cycles | | | | |

Comparison of additional key safety parameters indicated that 60 mg Q4W yielded similar safety profiles compared to 40 mg Q3W and 60 mg Q3W after 3-cycles (Table 13). Overall follow-up results indicated that 60 mg Q4W yielded lower overall infection (56%, 71%, 57%, and 43% at 20 mg Q3W, 40 mg Q3W, 60 mg Q3W, and 60 mg Q4W, respective) and had lower severity of infections (22%, 24%, 34%, and 10% Grade 3-4 infections at 20 mg Q3W, 40 mg Q3W, 60 mg Q3W, and 60 mg Q4W, respectively) (Table 13).

TABLE 13
Incidences for Cytopenias and Infections Overall and at 3-Month Follow-up

| | 20 mg | 40 mg | 60 mg | 60 mg |
|---|---|---|---|---|
| | Q3W | Q3W | Q3W | Q4W |
| | (N = 32) | (N = 55) | (N = 61) | (N= 21) |
| | Overall | Overall | Overall | Overall |
| | Follow-up | Follow-up | Follow-up | Follow-up |
| | (7.1 months) | (12.2 months) | (24.1 months) | (4.1 months) |
| Cytopenia | | | | |
| (Grade 3 or 4) | | | | |
| Neutropenia | 8 (25) | 17 (31) | 21 (34) | 3 (14) |
| Thrombocytopenia | 9 (28) | 9 (16) | 8 (13) | 2 (10) |
| Anemia | 8 (25) | 17 (31) | 8 (13) | 5 (24) |
| Infections | | | | |
| Overall (any grade) | 18 (56) | 39 (71) | 35 (57) | 9 (43) |
| Grade 3 or 4 | 7 (22) | 13 (24) | 21 (34) | 2 (10) |
| Serious | 5 (16) | 14 (25) | 20 (33) | 2 (10) |

To balance and equalize the comparison of the safety profile in the different cohorts, the incidence of adverse events (AEs) was evaluated using adjusted 3-cycle follow-up for each subject. When adjusted for duration of follow-up with 3-cycle analysis, the rate of Grade 3 or 4 neutropenia and thrombocytopenia is lower at 60 mg Q4W compared with 40 mg Q3W and 60 mg Q3W (Table 14). Additionally, incidence of Grade 3 or 4 infections and serious infections (all grades) in the 60 mg Q4W cohort is lower than the 40 mg Q3W and 60 mg Q3W cohorts, even when adjusted with 3-cycle analyses (Table 14).

TABLE 14
Incidences for Cytopenias and Infections at 3-Month Follow-up

| | 20 mg Q3W | 40 mg Q3W | 60 mg Q3W | 60 mg Q4W |
|---|---|---|---|---|
| | (N = 32) | (N = 55) | (N = 61) | (N= 21) |
| | 3-Cycle | 3-Cycle | 3-Cycle | 3-Cycle |
| | Analysis | Analysis | Analysis | Analysis |
| | (2.1 months) | (2.1 months) | (2.1 months) | (2.1 months) |
| Cytopenia | | | | |
| (Grade 3 or 4) | | | | |
| Neutropenia | 7 (22) | 10 (18) | 11 (18) | 3 (14) |
| Thrombocytopenia | 8 (25) | 7 (13) | 6 (10) | 1 (5) |
| Anemia | 7 (22) | 12 (22) | 8 (13) | 3 (14) |
| Infections | | | | |
| Overall (any grade) | 13 (41) | 21 (38) | 13 (21) | 7 (33) |
| Grade 3 or 4 | 3 (9) | 1 (2) | 5 (8) | 0 |
| Serious | 0 | 2 (4) | 5 (10) | 0 |

Incidence of immune-mediated toxicities, including CRS and ICANS, typically occurred on day 1 of the first cycle following the first maintenance dose of etentamig. Recurrence of CRS with subsequent infusion was rare with 0% at 60 mg Q4W, 4% at 40 mg Q3W (n=2), and 2% at 60 mg Q3W (n=1). In the 60 mg Q4W cohort, which implemented a modified premedication dose regimen, the overall incidence and severity of CRS were lower compared with the 40 mg Q3W and 60 mg Q3W cohorts. Notably, in the 60 mg Q4W cohort, the overall incidence was 43% with events that were Grade 1 or 2, with a total of 8 subjects (38%) experiencing Grade 1, 1 subject (5%) experiencing a Grade 2, and no subjects experiencing Grade ≥3 event (FIG. 2).

Therefore, the molecular structure of etentamig with low CD3 affinity coupled with the implementation of a modified premedication dose regimen in the 60 mg Q4W cohort effectively minimized the overall incidence of CRS (including Grade 1 and 2) and further mitigated Grade ≥3 events. 6.3 EXAMPLE 3-Administration of Etentamig 60 mg Q4W Is Safer And More Tolerable Than Other CD3×BCMA Bispecific Antibodies

This example demonstrates that etentamig 60 mg Q4W coupled with either implementation of a modified premedication dose regimen resulted in lower incidence and severity of CRS, as compared to other CD3×BCMA bispecific antibodies (e.g., Teclistamab, Elranatamab, Linvoseltamab).

In comparison to the CD3×BCMA bispecific antibodies teclistamab and elranatamab, the CRS incidence and severity observed with etentamig 60 mg Q4W is notably lower (Table 15). Of note, both teclistamab and elranatamab have implemented dosing regimens with two step-up doses each as well as a higher total amount of dexamethasone premedication in the first cycle in comparison to etentamig's 60 mg Q4W dosing regimen (Table 15). Additionally, few events of Grade 3 CRS were also observed with etentamig, as compared to both teclistamab and elranatamab, despite the 2 step-up dosing regimens and higher total amount of dexamethasone premedication in the first cycles for each of teclistamab and elranatamab. With etentamig, there were no Grade 3 events of CRS observed in the 60 mg Q4W cohort, with Grade 2 incidences lower comparatively at 5% without implementation of step-up dosing and with a modified premedication dose regimen that was lower than that with teclistamab or elranatamab.

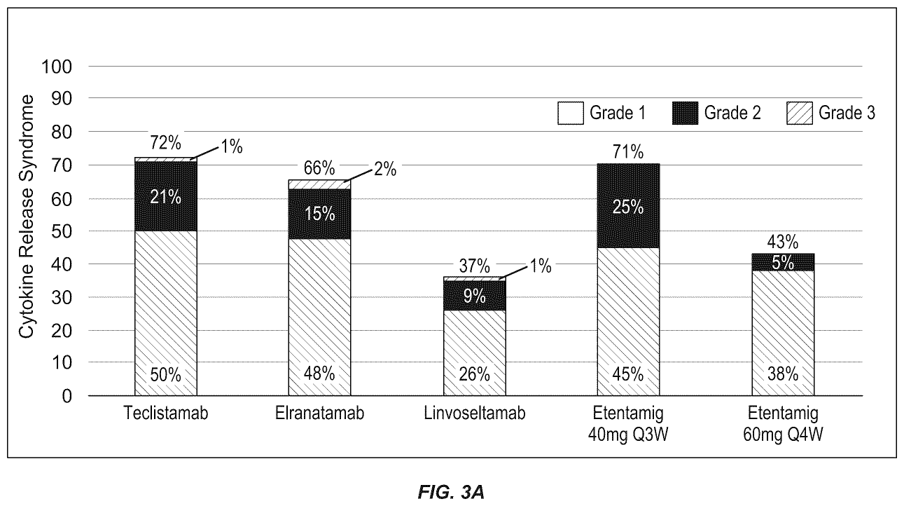

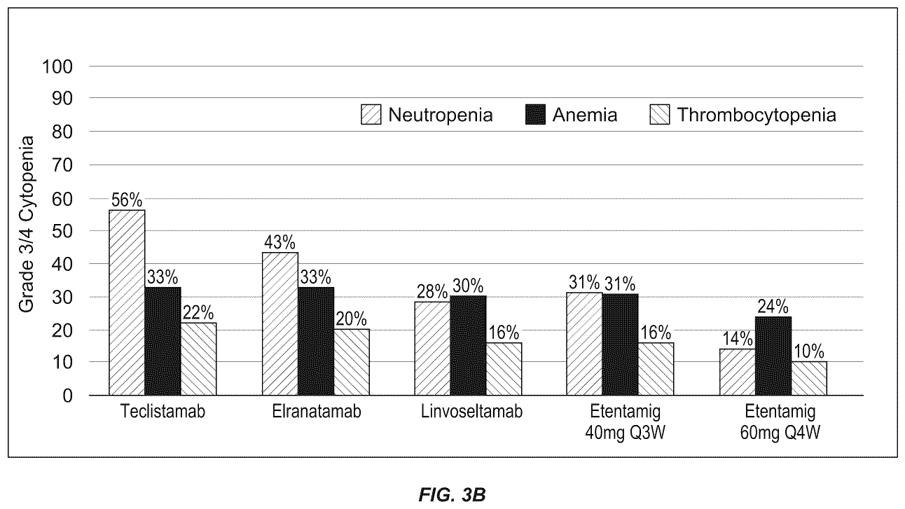

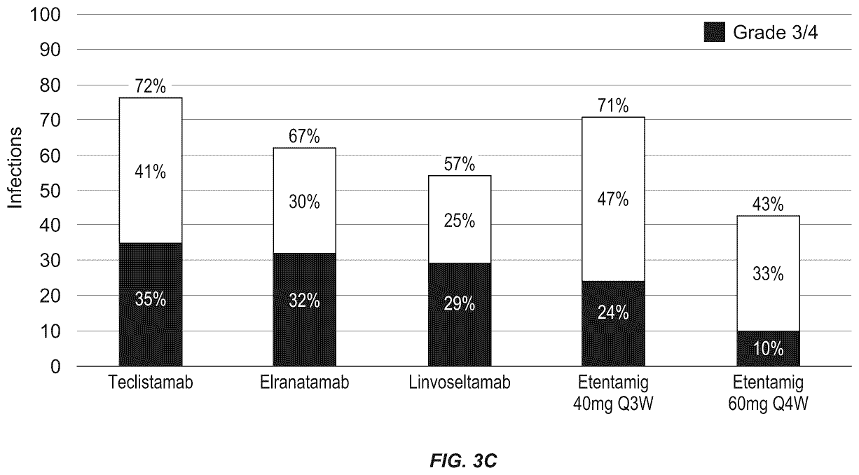

Furthermore, the safety profile for teclistamab and elranatamab involved a higher incidence (72%, 66%, respectively) and severity (50% Grade 1, 21% Grade 2, 1% Grade 3; 48% Grade 1, 15% Grade 2, 2% Grade 3, respectively) of CRS. (FIG. 3A). Grade 3/4 cytopenia (neutropenia, anemia, and thrombocytopenia) and infections for etentamig were each lower than teclistamab, elranatamab, and linoseltamab. (FIG. 3B and FIG. 3C).

The dosing regimens for teclistamab and elranatamab require that two step-up doses be given in the week prior to the first maintenance dose with weekly to weekly (for elranatamab, after 24 weeks based on response) dosing thereafter. Patients receiving teclistamab are required to remain within proximity of a healthcare facility (in the EU) or hospitalized (in the US) for 48 hours after administration of all doses within the first weekly dosing regimen (each of two step-up doses and one maintenance dose). Patients receiving elranatamab are required to be hospitalized for 48 hours after the first step-up dose, and 24 hours after the second step-up dose in the first cycle.

Thus, this example demonstrates that etentamig 60 mg Q4W could provide an efficacious, more convenient, more accessible, and potentially safer approach to selectively destroy BCMA-expressing MM cells, as compared to other CD3×BCMA bispecific antibodies.

TABLE 15
Comparison of Etentamig 60-mg Q4W Dosing and Administration with Teclistamab
and Elranatamab Dosing and Administration Labeling Information

| | Etentamig | Teclistamab | Elranatamab |
|---|---|---|---|
| Regimen | Q4W | Q1W | Q1W |
| | | | (may extend to Q2W |
| | | | after 24 weeks per |
| | | | response) |
| Route of | IV | SC | SC |
| Administration | | | |
| CD3xBCMA | Day 1: Maintenance | Day 1: Step-up dose 1 | Day 1: Step-up dose 1 |
| bispecific antibody | dose 60 mg (no step- | 0.06 mg/kg | 12 mg |
| Dosing in Cycle 1 | up dose) | Day 4: Step-up dose 2 | Day 4: Step-up dose 2 |
| | | 0.3 mg/kg | 32 mg |
| | | Day 7: Maintenance | Day 8: Maintenance |
| | | dose | dose 76 mg |
| | | 1.5 mg/kg | |
| Dexamethasone | Total 36 mg | Total 48 mg | Total 60 mg |
| Premedication | (8 mg PO 12 | (16 mg prior to each | (20 mg prior to each |
| in Cycle 1 | hours pre-dose; | dose including step- | dose including step- |
| | 8 mg PO/IV 3-4 | up dose) | up dose) |
| | hours pre-dose; | | |
| | 20 mg IV/PO 15-60 | | |
| | minutes pre-dose) | | |
| Rate of CRS | Overall incidence: 43% | Overall incidence: 72% | Overall incidence: 58% |
| | Grade 1: 38% | Grade 1: 50% | Grade 1: 44% |
| | Grade 2: 5% | Grade 2: 21% | Grade 2: 14% |
| | Grade 3: 0% | Grade 3: 1% | Grade 3: 1% |

### 6.4 EXAMPLE 4-Pharmacokinetic And Exposure-Response Analysis Of Etentamig Support 60 mg Q4W Maintenance Dose

This example demonstrates that the extended dosing interval of Q4W for the maintenance dose of 60 mg (without a step-up dose) is supported by the long half-life of etentamig of 17-27 days from population pharmacokinetic (popPK) models and exposure-response (safety and efficacy) analyses.

To determine the pharmacokinetic characteristics of etentamig, serial blood samples were collected in cycles 1, 3, and 6, along with sparse samples throughout the study from dose escalation (0.025-120 mg Q3W) and dose expansion phases (20 mg Q3W, 40 mg Q3W, 60 mg Q3W, and 60 mg Q4W-all without a step-up dose). Etentamig serum total (unbound and bound to soluble BCMA) and free (unbound and partially bound to soluble BCMA) concentrations were determined using the validated LC-MS method (LLOQ of 99 ng/mL) and ligand binding assay (LLOQ of 20 ng/mL), respectively. Data for antidrug antibodies (ADA) were available from 204 evaluable subjects. The serum concentrations for 0.025 mg (n=3) and 0.075 mg (n=3) were below the LLOQ at all pharmacokinetic (PK) time points for noncompartmental analysis (NCA). Intrasubject dose escalations (ISDEs) were allowed in the dose-escalation cohorts and the 20 mg expansion cohort. For subjects with ISDEs, the PK data collected after first intrasubject dose escalation (ISDE) were excluded from NCA.

Preliminary PK results suggested that total and free etentamig exhibited approximately dose-proportional PK following single doses of 5.4 mg to 120 mg. The PK profile and results from the available PK data (total etentamig, n=19 and free etentamig, n=20) for 60 mg Q4W are summarized in Table 16. The estimated terminal phase half-life for total and free etentamig was 13.5 days and 12 days, respectively. Steady state was achieved by Cycle 4 after dose administration at Q3W frequency in the expansion cohorts. Estimated clearance (CL) and volume of distribution at steady state (Vss) were consistent with those of a typical IgG4 antibody in humans. Accumulation (cycle 3 vs cycle 1) was 48% to 102% and 59% to 76% for total and free etentamig exposures (Cmax and Ctrough), respectively, following multiple doses of 60 mg Q4W dosing regimen. Etentamig total AUCtau was 46% higher than that representing free etentamig with no/minimal observed differences in Cmax and Ctrough (≤13%). Preliminary assessment of immunogenicity indicated an ADA incidence of 3.9% (8 of 204), with low titers ranging between 11.7 and 187 titer units and no impact on PK (Table 16).

TABLE 16
PK Parameters (Geometric Mean [Mean, % CV])
of Etentamig After 60 mg Q4W Dosing in Cycle 1

| | Free etentamig | Total etentamig |
|---|---|---|
| Parameter | (N = 20) | (N = 19) |
| Dose proportionality | Approximately dose-proportional | |
| | PK (5.4 mg-120 mg Q3W) | |
| Cₘₐₓ | 14.9 | 16.9 |
| (μg/mL) | (16.2, 49) | (17.4, 25) |
| AUCₜₐᵤ | 102.5 | 150 |
| (μg · day/mL)^{a} | (124.3, 53) | (160, 32) |
| C_{trough} (μg/mL)^{b} | 2.54 | 2.55 |
| | (2.64, 56) | (3.05, 37) |
| t½ (day)^{c} | 12.0 ± 10.4 | 13.5 ± 8.0 |
| CL (L/day) | 0.51 | 0.40 |
| | (0.61, 81) | (0.44, 52) |
| V_{SS} (L) | 10.0 | 8.27 |
| | (11.2, 47) | (8.96, 43) |
| Steady State | Appears to reach by Cycle 4 (Q3W) | |
| Accumulation in Cₘₐₓ | 59%-76% ↑ | 48%-102% ↑ |
| and C_{trough} (Cycle 3 | | |
| v. Cycle 1 Q4W) | | |

| | |
|---|---|
| Free vs total | Total > Free |
| (Cycle 1) | (46% higher AUC; Similar Cₘₐₓ and C_{trough} |
| | [≤13% higher]) |
| Immunogenicity | Low ADA incidence of 3.9% (8/204) |

The geometric mean total and free Ctrough values after first treatment dose with 60 mg Q4W (Table 16) were approximately 2-fold higher than the maximum EC90 (1.29 μg/mL) identified in ex vivo cytotoxic assays. Given the observed accumulation following multiple doses during treatment, the Ctrough values with 60 mg Q4W are expected to be maintained multifold higher than the maximum cytotoxic assay EC90 throughout the treatment.

Two separate popPK models for etentamig were developed using available total and free PK data representing 215 subjects from the Phase 1 Study. The popPK models were built using a nonlinear mixed-effects modeling approach by employing NONMEM 7.5 software. Both the total and free PK of etentamig were characterized by 2-compartment models. A mean terminal half-life (t1/2,β) of etentamig was derived using clearance (CL and Q) and volume (Vc and Vp) population parameter estimates from both total and free popPK models. The mean t1/2,β was approximately 27 days for total etentamig and 17 days for free etentamig in the RRMM patients. A steady-state volume of distribution was calculated as a sum of the central (Vc) and peripheral (Vp) volumes of distribution and was found to be approximately 7.7 L and 7.9 L for total etentamig and free etentamig, respectively. Post hoc parameter estimates (empirical Bayes estimates) from the final models, individual dosing histories, and covariate effects were used to derive the following model based on total and free PK exposures for ER analyses: (1) Average concentration over the concentration-time curve (Cavg) up to the first best overall response or up to the first safety endpoint of interest; and (2) Average Ctrough (avgCtrough) up to the first best overall response or up to the first safety endpoint of interest.

Exposure-response (ER) analyses for efficacy and safety endpoints were conducted separately for total and free PK exposures. The efficacy (ORR [PR+VGPR+sCR/CR], >VGPR [VGPR+sCRCR], and sCR/CR) and safety (Grade ≥3 TEAEs, TE-SAEs, serious infections, Grade ≥3 infections, overall infections, any-grade CRS, Grade 1 only CRS, Grade 2 only CRS, and Grade ≥2 CRS) endpoints using observed Cycle 1 total and free PK exposures (AUCinf, Cmax, and Ctrough [except for all safety endpoints]) and the above model-derived exposures (Cavg [except for CRS endpoints] and avgCtrough [except for all safety endpoints]) were modeled using logistic regression methodology (using R Version 4.2.2).

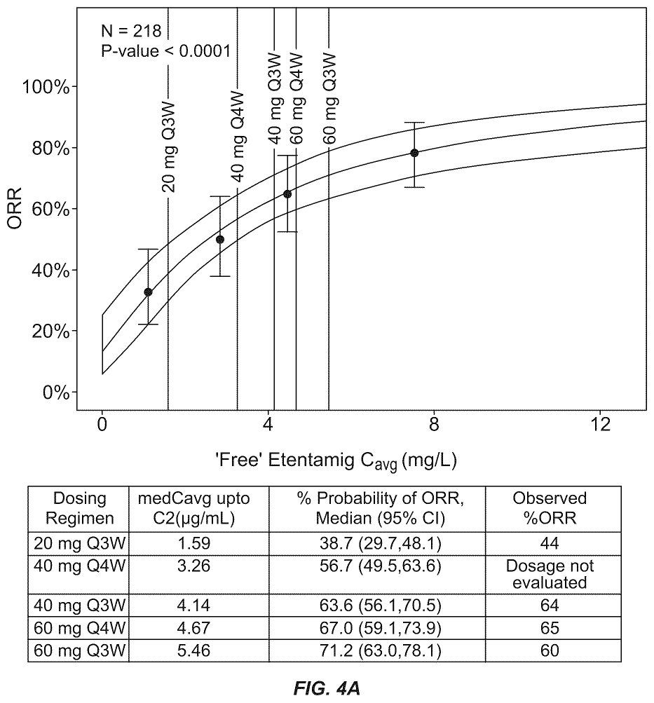

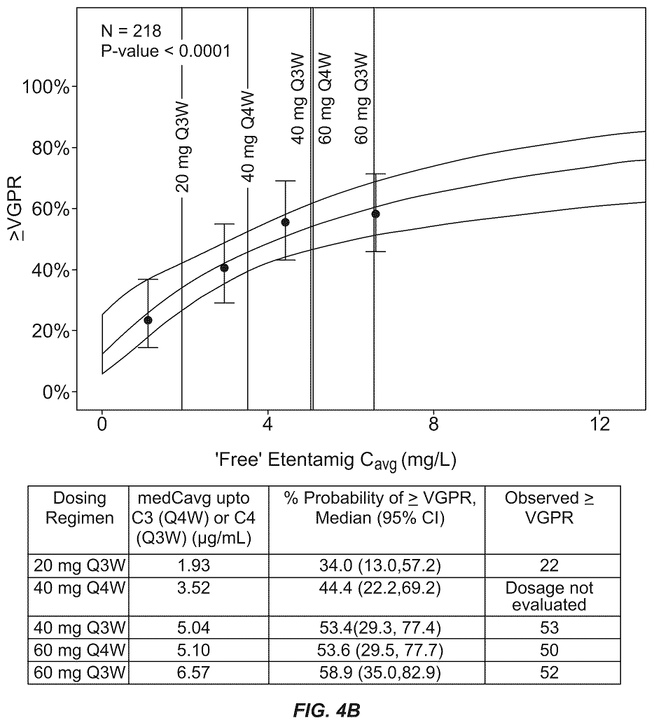

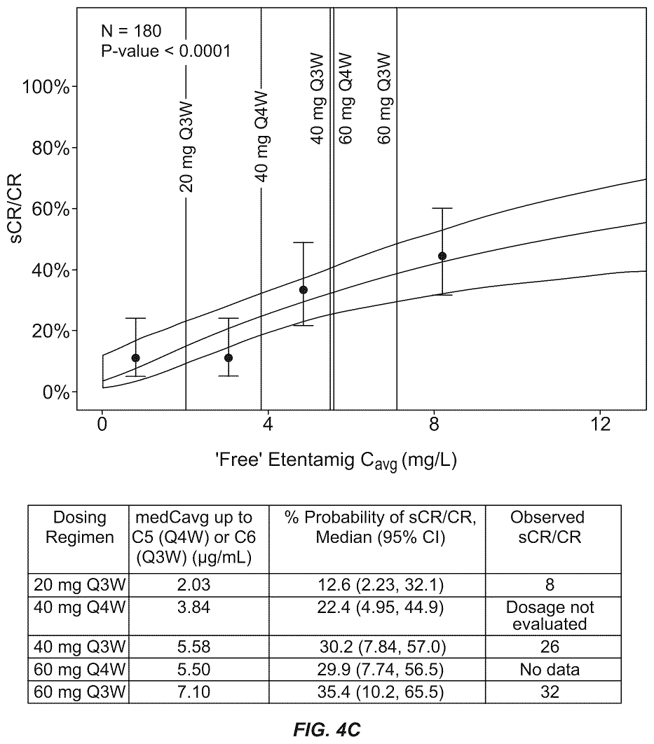

Based on overall assessment of all models with different PK exposures (total or free) and different PK metrics, it was observed that the efficacy endpoints (ORR, >VGPR, and sCR/CR) had relatively strong correlation with free etentamig PK exposure metrics. This observation is consistent with the understanding and relevance of free PK exposure (pharmacologically active component) with respect to response. The ER-efficacy relationships with free etentamig Cavg PK metric are used to justify the dose. Exposure-efficacy relationships of ORR (n/N=123/218), >VGPR (n/N=97/218), and sCR/CR (n/N=45/180) with free etentamig Cavg exposure metric suggest that the probability of achieving response increases with increasing Cavg (FIG. 4A, FIG. 4B, and FIG. 4C). In FIGS. 4A-4C, data in solid symbols, the binomial 95% CI of the observed data as error bars, prediction from the logistic regression model as a solid black line, and 95% CI of the prediction in shaded region. The vertical light blue lines indicate the median simulated exposure for the respective dose level. No covariate was selected in covariate search for ORR ER model. Only IgG MM disease type was selected for >VGPR and sCR/CR ER models. None of the other tested covariates were selected for >VGPR and sCR/CR ER models.

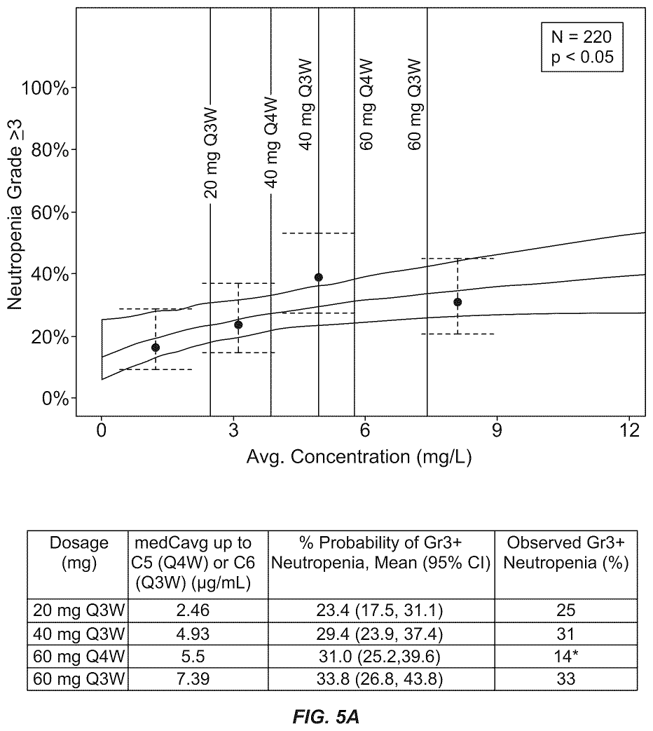

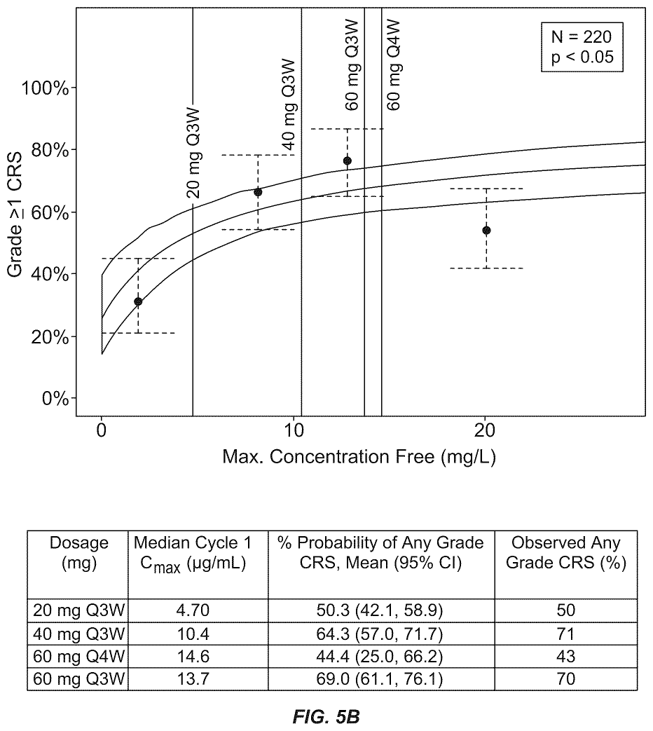

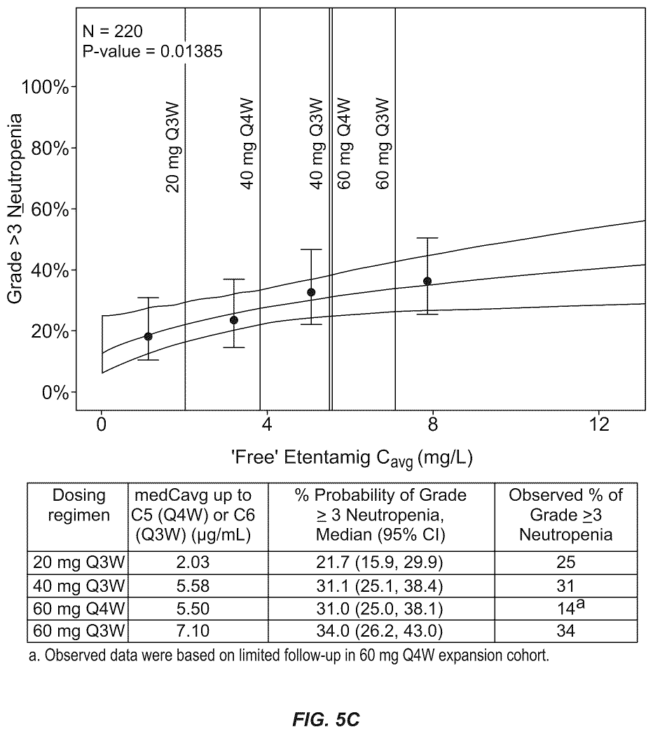

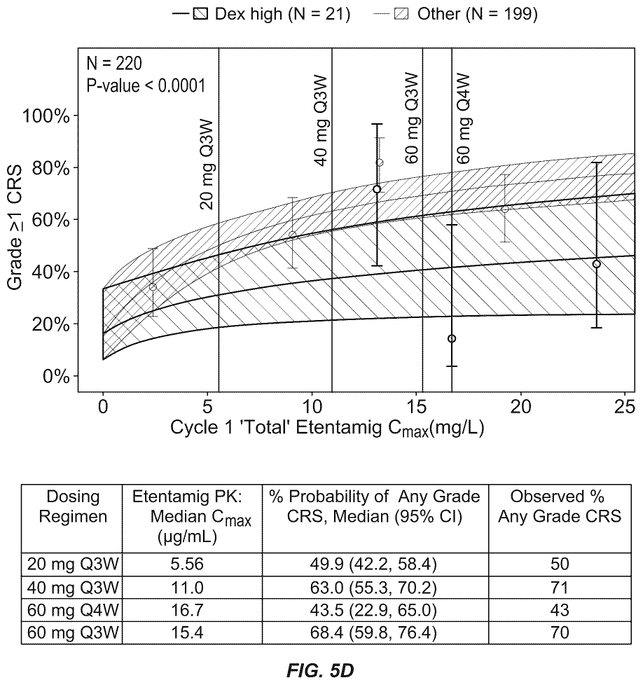

Based on overall assessment of all models with different PK exposures (total or free) and various PK metrics, it was observed that any-grade CRS and Grade 1 only CRS had relatively strong correlation with cycle 1 total etentamig Cmax, which is also a most relevant PK exposure metric given that the CRS events primarily occurred after first administration on day 1 of cycle 1. Overall, the free etentamig Cavg appeared to consistently be one of the relatively strongly correlated exposure metrics for Grade ≥3 neutropenia using a first and second PK simulation (FIG. 5A and FIG. 5C, respectively).

Exposure-safety relationships of any-grade CRS (n/N=125/220), Grade 1 only CRS (n/N=85/220), and Grade ≥3 neutropenia (n/N=61/220) with their respective exposure metric (Cycle 1 total Cmax or free Cavg) suggest that the probability of occurrence of the safety event increases with increasing exposure (FIG. 5A-FIG. 5D). These results were consistent using a first PK simulation (FIG. 5A and FIG. 5B) and second PK simulation (FIG. 5C and FIG. 5D). None of the tested covariates were selected in these safety ER models. The subjects in the 60 mg Q4W expansion cohort received modified dexamethasone premedication dose regimen (a total dose of 36 mg) and had relatively lower incidence of CRS events (43%, any-grade CRS) compared with those subjects in the 40 mg Q3W and 60 mg Q3W expansion cohorts (˜70% any-grade CRS) who received standard dexamethasone premedication (a total dose of 10 mg) in this Study. The dexamethasone premedication dose (modified vs standard) was hence included as a predictor in the CRS ER models.

In summary, the exposure-response (safety and efficacy) analyses suggest that the 60 mg Q4W dose with modified premedication dose regimen provides a better CRS profile and similar Grade ≥3 neutropenia profile compared with the 40 mg Q3W and 60 mg Q3W doses, while maintaining efficacy, with 67% ORR, 54%>VGPR, and 30% sCR/CR. Additionally, the estimated half-life (t1/2,β) of 17 to 27 days from population pharmacokinetic(s) models and the expected Ctrough values above maximum EC90 from ex vivo cytotoxic assays support the Q4W dosing frequency, an extended dosing interval that offers convenience to patients. Thus, the PK and ER analyses support 60 mg Q4W with modified premedication dose regimen as the optimal etentamig monotherapy maintenance dose.

Taken together, the above data provided in the Examples indicate that etentamig showed a manageable safety profile at dosages of 20, 40, and 60 mg IV Q3W and 60 mg Q4W, with low incidence of treatment-emergent adverse events (TEAEs) leading to discontinuation. Lower incidence (43%) and severity (Grade [G]1 38%/G2 5%) of CRS that was reported at 60 mg Q4W may indicate contribution from the modified premedication dose regimen. At 40 mg and 60 mg, etentamig monotherapy yielded deep and durable responses with median progression-free survival (PFS) of 13.7 and 11.2 months, and 12-month duration of response (DOR) of 70% and 66% (median DOR not reached in patients with >complete response [CR]), respectively.

### 6.5 EXAMPLE 5-Population Pharmacokinetic (PK) Models Describe the Total and Free PK Data of Etentamig

This example demonstrates that two compartment popPK models adequately described total and free etentamig PK data. In particular, separate population pharmacokinetic models were developed using total PK data, which represents etentamig concentrations unbound and bound to soluble BCMA, and free PK data, which represents unbound and partially bound to soluble BCMA. Patient-specific covariates affecting total and free PK exposures were also identified.

Two separate popPK models for etentamig were developed using available total and free PK data representing 215 subjects from the Phase 1 Study, as described in Example 4, for the maintenance dose of 60 mg (without a step-up dose). Model development and evaluation included diagnostic plots, likelihood ratio tests (p<0.01; x2 distribution) and prediction-corrected visual predictive checks (VPCs). Briefly, etentamig serum total and free PK data (n=215) from a Phase 1 dose escalation and expansion study were used to build two separate nonlinear mixed-effects models in NONMEM 7.5. The dataset consisted of 6093 total and 6085 free concentrations from dosing cohorts of 0.025 mg Q3W to 120 mg Q3W and 60 mg Q4W-all without a step-up dose. Patient-specific covariates (demographics, baseline albumin, baseline sBCMA, time varying sBCMA, renal/hepatic functions, baseline disease/performance characteristics, and number of prior therapies) were tested on relevant clearance and volume parameters.

The results indicated that etentamig total and free PK were adequately described by two compartment models with linear elimination. The clearance (CL) estimates of total and free etentamig were 0.205 L/day and 0.346 L/day with inter-individual variabilities (% CV) of 49% and 78%, respectively. The mean terminal half-lives (t1/2,β) and steady-state volumes of distribution (Vss) derived using the popPK model estimated clearance (CL and Q) and volume (Vc and Vp) were approximately 27 days and 7.7 L for total etentamig and 17 days and 7.9 L for free etentamig, respectively. The clearance and volume parameters were allometrically scaled using body weight. Parameter estimates for total and free etentamig population pharmacokinetic models are provided in Table 17.

TABLE 17
Parameter Estimates for Total and Free Etentamig
Population Pharmacokinetic Models

| | | |
|---|---|---|
| Parameter | Free etentamig | Total etentamig |

Pop. Est. (95% CI)

| | | |
|---|---|---|
| CL (L/day) | 0.205 | 0.346 |
| | (0.180, 0.234) | (0.314, 0.380) |
| Vc (L) | 3.67 | 3.94 |
| | (3.53, 3.81) | (3.75, 4.14) |
| Q (L/day) | 1.33 | 1.39 |
| | (1.17, 1.51) | (1.28, 1.151) |
| Vp (L) | 4.03 | 3.94 |
| | (3.71, 4.36) | (3.79, 4.11) |
| BW on CL and Q | 0.492 | 0.750 |
| | (0223, 0.761) | (fixed) |
| BW on Vc and Vp | 0.501 | 1.00 |
| | (0.406, 0.596) | (fixed) |
| Baseline sBCMA on CL | 0.129 | 0.343 |
| | (0.0704, 0.187) | (0.263, 0.423) |
| Baseline sBCMA on Vc | 0.0647 | 0.256 |
| | (0.0449, 0.0845) | (0.214, 0.297) |
| Sex (Female) on CL | 0.781 | — |
| | (0.675, 0.905) | |
| Sex (Female) on Vc | 0.852 | — |
| | (0.810, 0.897) | |
| Myeloma Type IgG | 1.31 | — |
| on CL | (1.15, 1.48) | |

Pop. Est. (% CV)

| | | |
|---|---|---|
| IIV on CL | 0.215 | 0.476 |
| | (49) | (78) |
| IIV on Vc | 0.0229 | 0.124 |
| | (15) | (36) |
| Covariance of CL | 0.033 | — |
| and Vc | (18) | |
| IIV on Q | 0.382 | — |
| | (68) | |
| IIV on Vp | 0.240 | — |
| | (52) | |
| Covariance of Q | 0.030 | — |
| and Vp | (17) | |

Pop. Est. (% RSE)

| | | |
|---|---|---|
| Proportional Error | 0.0292 | 0.0518 |
| | (2.0) | (2.0) |
| Additive Error | 0.00274 | 0.00162 |
| | (17) | (15) |

BW = body weight;
CI = confidence interval;
CL = clearance;
IgG = immunoglobulin G;
IIV = inter-individual variability;
Q = intercompartmental clearance;
sBCMA = soluble B-cell maturation antigen;
Vc = Volume of central compartment;
Vp = volume of peripheral compartment
Note:
% Relative standard error (% RSE) was calculated as the standard error divided by the estimate multiplied by 100. % Coefficient of Variation (% CV) = SQRT(exp(ω2)−1)*100; 95% CI was approximated as the point estimate ± 1.96* standard error of estimate.

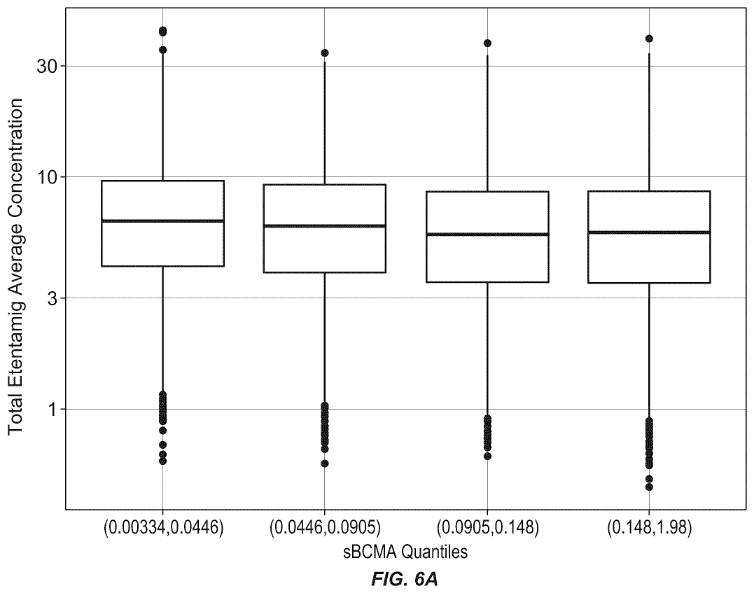

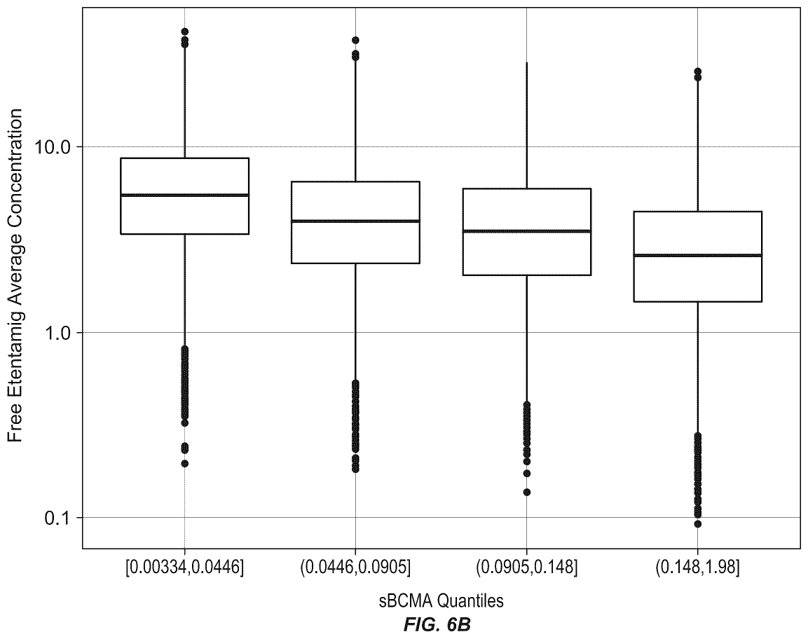

Sex, baseline soluble BCMA, and IgG multiple myeloma disease sub-type were significant covariates in total PK model. Only baseline soluble BCMA was a significant covariate in free PK model. Although covariates influenced total PK, the effects on free PK were considered most relevant given the pharmacological relevance of free etentamig. As shown in FIG. 6A and FIG. 6B, the baseline soluble BCMA covariate effects in total (FIG. 6A) and free (FIG. 6B) population PK models suggest that higher baseline soluble BCMA levels reduce free PK exposures only and do not impact total PK exposures.

Taken together, the data provided in the Examples indicate that the 2-compartment models adequately described the total and free PK data of etentamig. Higher baseline soluble BCMA levels reduce free PK exposures only and do not impact total PK exposures.

### 6.6 EXAMPLE 6-Correlative Biomarker Analysis for Therapeutic Dose Determination of Etentamig

This example demonstrates that correlative biomarker results of baseline and longitudinal soluble B-cell maturation antigen (sBCMA) levels and immune profiles support 60 mg Q4W etentamig monotherapy maintenance dose (without a step-up dose).

Peripheral blood and serum samples from patients with RRMM who received etentamig at 20, 40, or 60 mg Q3W or 60 mg Q4W were collected at baseline (post-dexamethasone, pre-etentamig), on treatment, and at disease progression. Samples were analyzed by flow cytometry for immune cell populations, Luminex for cytokines, and electrochemiluminescence ligand binding for sBCMA.

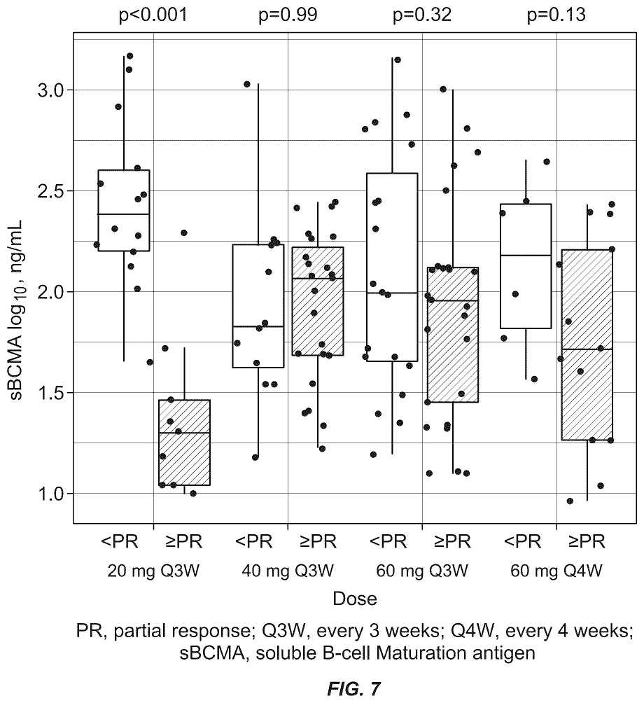

Median sBCMA levels at baseline (post-dexamethasone, pre-etentamig) were highly variable among patients but did not associate with clinical response (>partial response [PR]) to etentamig at the selected dose level of 60 mg Q4W. As shown in FIG. 7, median baseline sBCMA levels in the 60 mg Q4W etentamig cohort (rightmost pair of box plots) were 244.7 ng/mL for nonresponders (<PR) and 52.2 ng/ml for responders (≥ PR) (p=0.13).

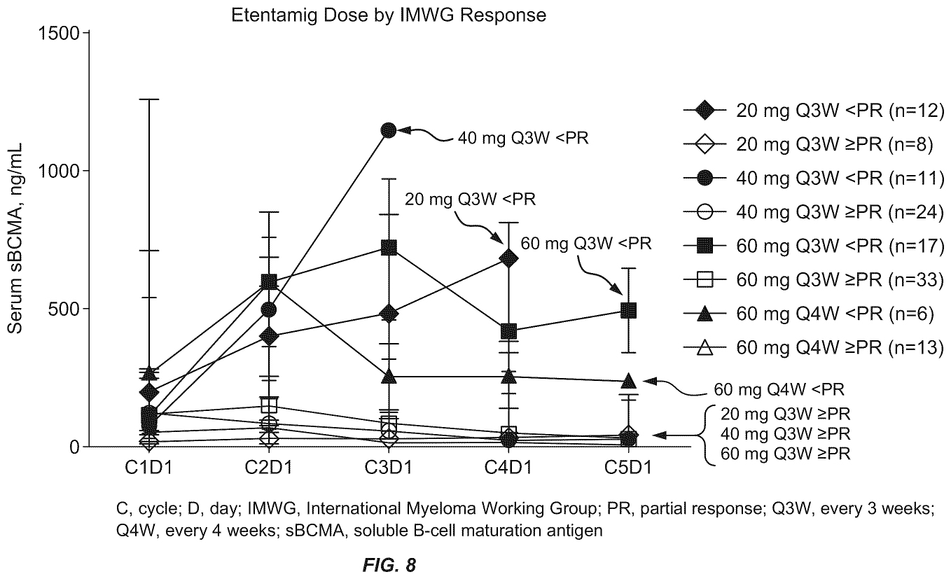

Reduction in sBCMA levels over time was associated with clinical response. As shown in FIG. 8, at 60 mg Q4W etentamig, the minimum serum concentrations of sBCMA at cycle 5 were 244.7 ng/ml for nonresponders (<PR; uppermost four lines) and 4.7 ng/ml for responders (>PR; lowermost four lines) (p=0.04).

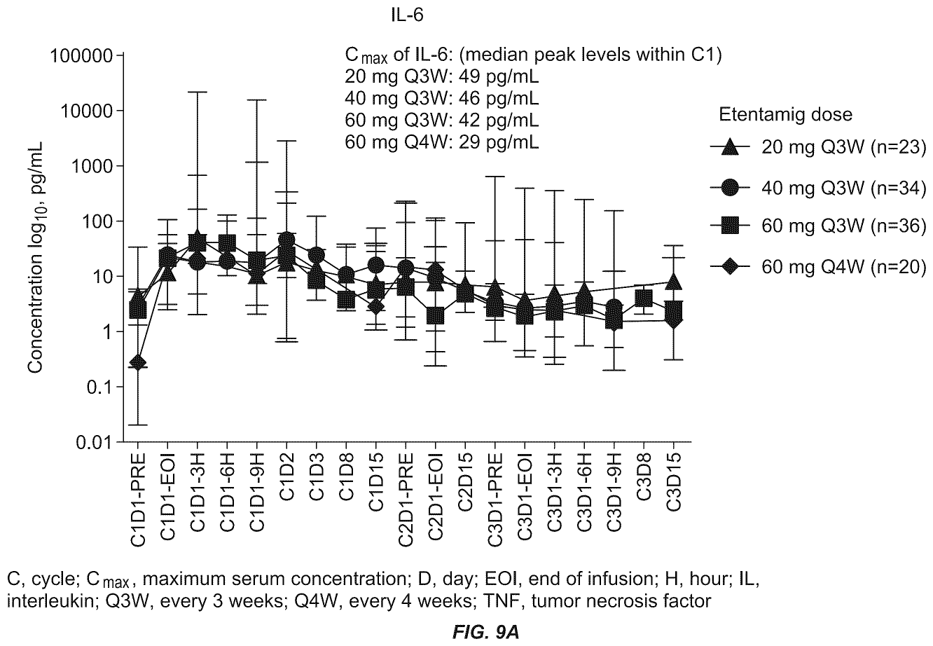

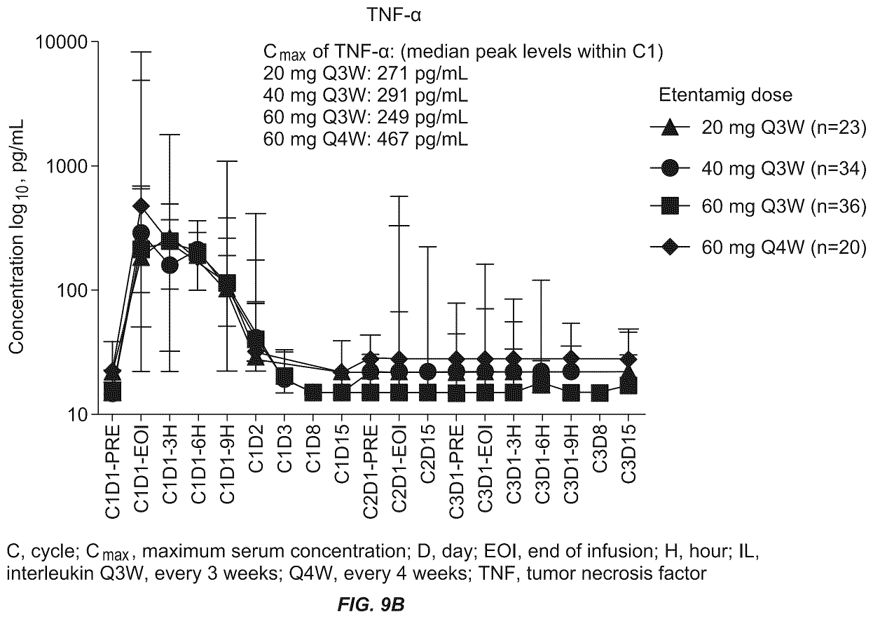

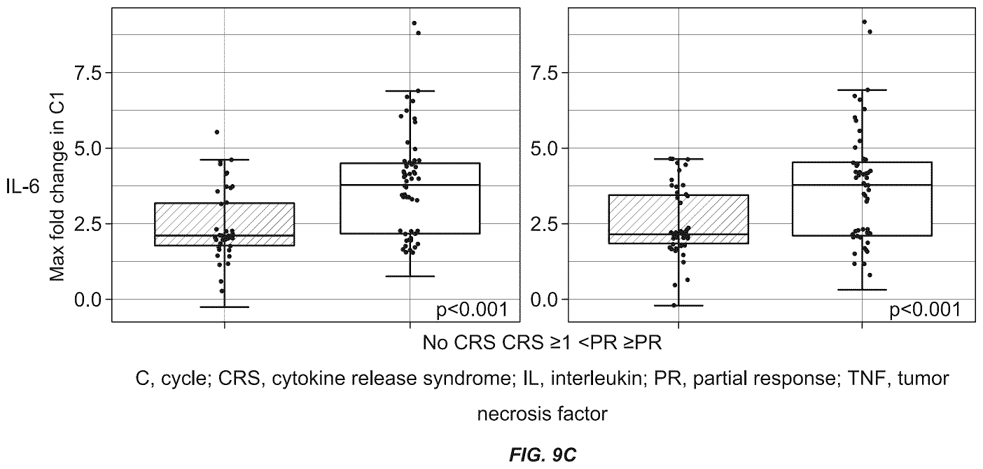

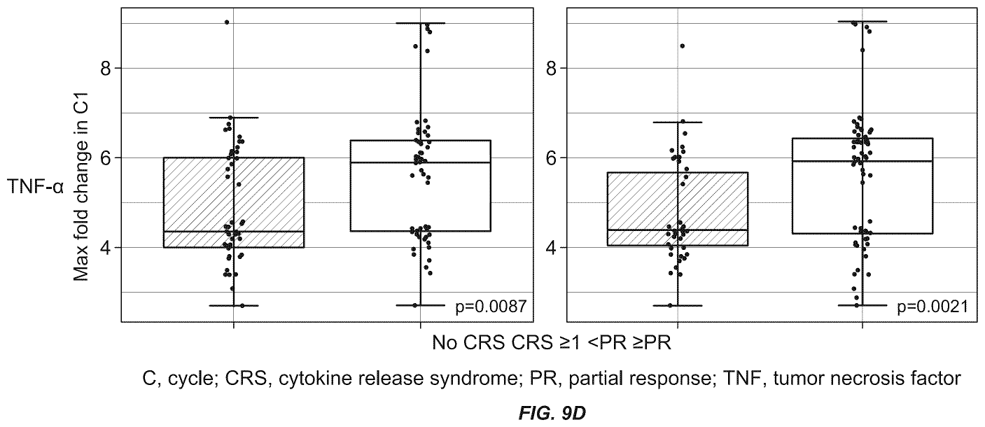

Treatment with etentamig resulted in a rapid and transient increase of proinflammatory cytokines. As shown in FIG. 9A, following treatment with 60 mg Q4W of etentamig, proinflammatory cytokine maximum serum concentrations within cycle 1 peaked at 29 μg/mL for IL-6 (FIG. 9A); 456.5 pg/mL for TNF-α (FIG. 9B); and 349.5 pg/mL for IL-8 and 1375 μg/mL for IL-10 (analyzed but not pictured). Further, peak induction levels of the proinflammatory cytokines IL-6 (FIG. 9C) and TNFα (FIG. 9D) during cycle 1 correlated with the occurrence of any grade of CRS (>G1) (FIG. 9C and FIG. 9D, left) and with clinical response across dose levels (FIG. 9C and FIG. 9D, right).

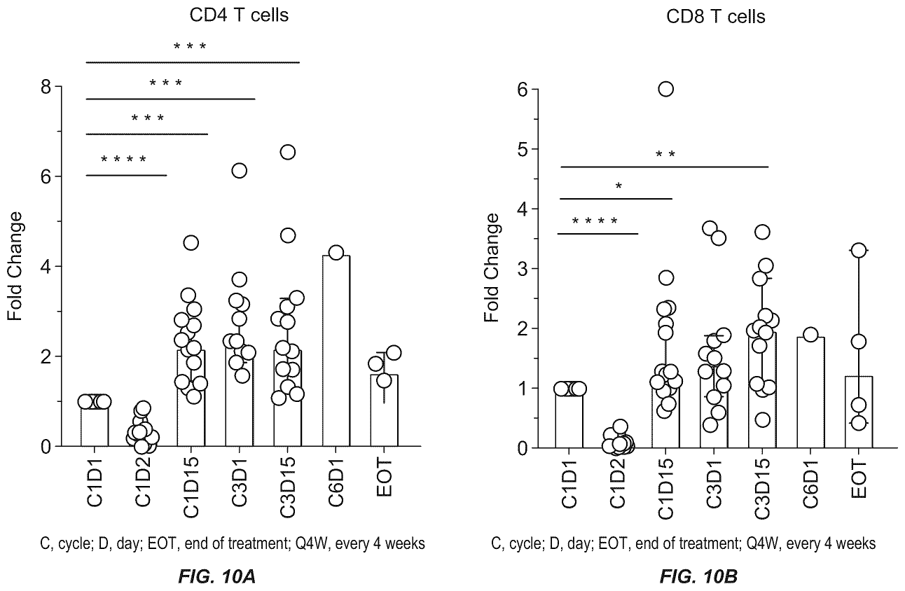

Treatment with etentamig at 60 mg Q4W promoted T-cell redistribution and expansion. As shown in FIG. 10, both CD4 T-cells (left) and CD8 T-cells (right) demonstrated redistribution, with an 89% reduction of peripheral CD8+ T-cells at 60 mg Q4W observed in cycle 1 day 2 (FIG. 10 right). T-cell expansion was only observed with etentamig at 60 mg Q4W. Further, treatment with etentamig at 60 mg Q4W resulted in T-cell activation (a 1.9-fold increase in CD69+CD8+ T cells) and proliferation (a 2-fold increase in Ki67+CD8+ T cells) within cycle 1.

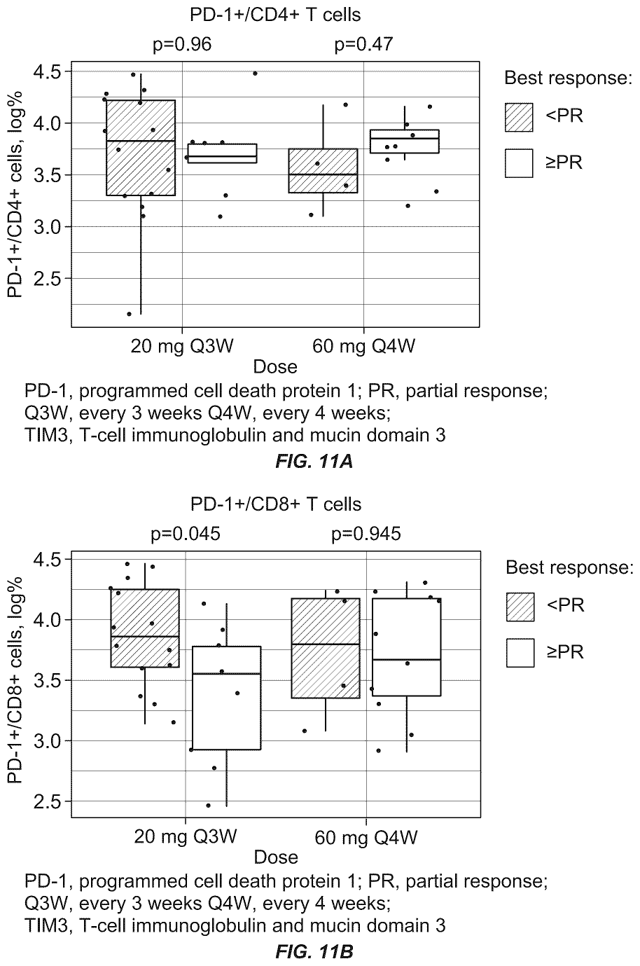

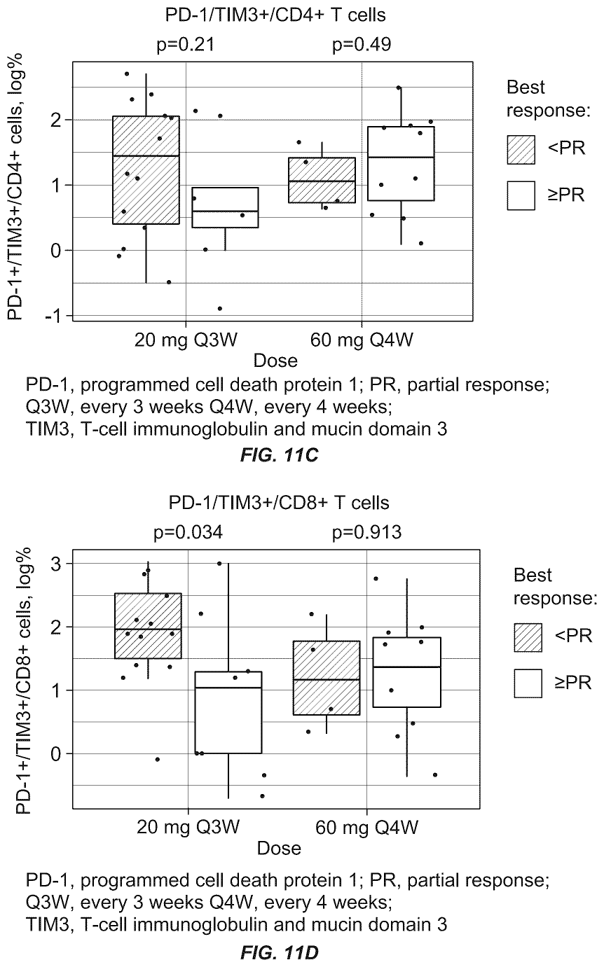

The frequency of baseline T-cell exhaustion did not impact clinical response for etentamig at 60 mg Q4W. As shown in FIG. 11, the frequency of CD8+ T-cell exhaustion, as measured by PD-1/TIM3 ratio (FIG. 11, right column) did not impact the clinical response at the 60 mg Q4W etentamig treatment condition (FIG. 11 lower right) (<PR: 3.5%; ≥PR: 3.9%; P=0.9).

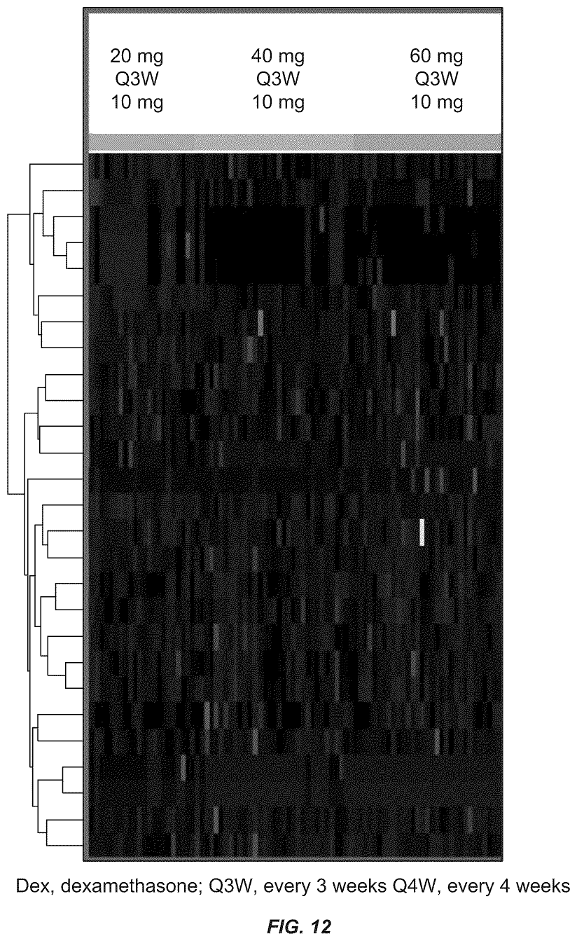

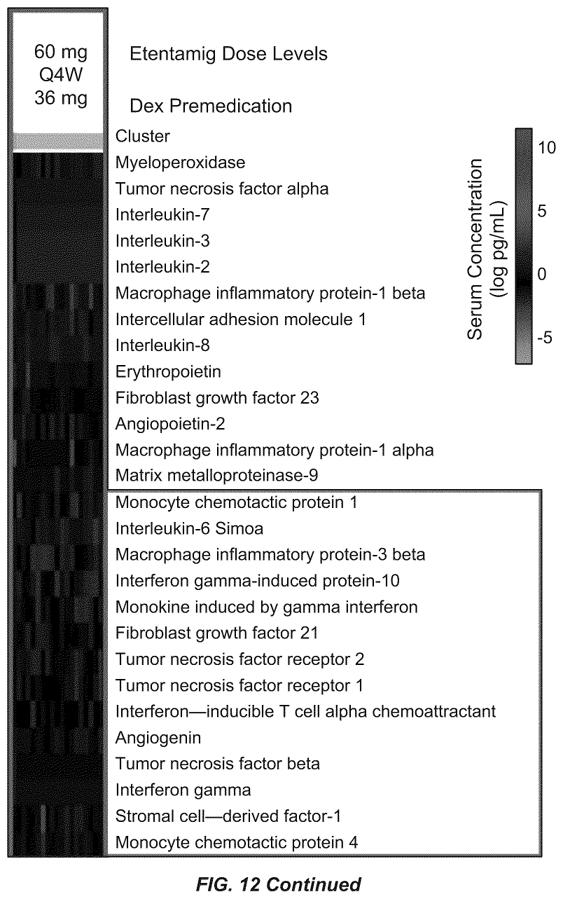

Including a modified premedication dose regimen with a total 36 mg dexamethasone in the 60-mg Q4W cohort reduced the baseline serum levels of cytokines and chemokines pre-etentamig administration. As shown in FIG. 12, serum concentration measurements indicate that utilizing the modified premedication dose regimen with a total 36 mg dexamethasone (rightmost vertical rectangle) resulted in lower expression of myeloid-derived inflammatory markers (lower right box).

Taken together, the results provided in this example demonstrate that correlative biomarker results of baseline and longitudinal sBCMA levels and immune profiles supports 60 mg Q4W etentamig monotherapy. High-avidity BCMA binding coupled to low-affinity T-cell engagement distinguishes the resulting mechanism of action for etentamig. Responses to 60 mg Q4W etentamig were independent of baseline sBCMA levels and resulted in rapid and transient proinflammatory cytokine production that promoted robust T-cell redistribution, activation, and proliferation, indicating that etentamig at 60 mg Q4W maximizes its clinical potential as a convenient, safe, and effective therapy for MM.

### 6.7 EXAMPLE 7-Severity and Incidences of CRS are lowered by introducing Step-up Dosing of Etentamig

This Example demonstrates that administering a step-up dose of etentamig before a maintenance dose of etentamig results in lower instances and severity of CRS and similar instances of ICANS, as compared to (1) administering etentamig without a step-up dose, or (2) administering etentamig without a a modified premedication dose regimen.

Patients with relapsed/refractory multiple myeloma (RRMM) who have received at least 3 prior lines of therapy, including exposure to a proteasome inhibitor (PI), an immunomodulatory imide drug (IMiD), and an anti-CD38 monoclonal antibody were enrolled to evaluate the safety and efficacy of optimization (Part 1-1 SUD+1 maintenance dose) and expansion (Part 2-See Table 18). Median patient age was 69 years (range 40-84) and 41 (59%) of patients were male. The majority of patients were White (n=56, 81%); 10 (14%) were Black or African American, three (4%) were Asian and one (1%) patient's race was not reported. The majority of patients were treated in North America (n=41, 59%), with 20 (29%) patients being treated in Asia and the remaining nine (13%) patients were treated elsewhere. Baseline Eastern Cooperative Oncology Group Performance Status (ECOG PS) was 0 for 24 (34%) patients, 1 for 44 (63%) patients, and 2 for two (3%) patients. Revised-International Staging System (R-ISS) score at study entry was I in 16 (23%) patients, II in 38 (54%) patients, III in 12 VAI-1541987118 73 (17%) patients, and unknown in four (6%) patients. Patients had received a median of four (range 3-10) prior lines of therapy. Thirty-nine (56%) patients were relapsed to prior cancer therapy and 31 (44%) were refractory. Fifty-three (76%) patients were refractory to prior triple-class cancer therapy (refractory to PI, IMID and anti-CD38 antibody) and 22 (31%) were refractory to penta-drug (refractory to at least two PIs, at least two IMIDs, and at least one anti-CD38 antibody) prior cancer therapy.

In Part 1, patients received a single step-up dose of etentamig (2 mg or 4 mg) on cycle 1 day 1 (C1D1) (intravenously), followed by a maintenance dose of 60 mg on day 4 (D4). In subsequent cycles (>cycle 2), subjects received a 60 mg dose of etentamig administered IV on day 1 of each cycle Q4W. In Part 1, patients were premedicated with 10 mg dexamethasone orally, 15 to 60 minutes prior to the step-up dose of etentamig infusion on cycle 1 day 1 (C1D1), and patients received a 10 mg oral dexamethasone 15 to 60 minutes prior to the maintenance dose of etentamig infusion on cycle 1 day 4 (C1D4).

In Part 2, patients received 2 mg etentamig on CID1 as a step-up dose, followed by 60 mg on C1D4. Patients were premedicated with 10 mg dexamethasone orally, 15 to 60 minutes prior to the step-up dose of etentamig infusion on C1D1. During Part 2, on C1D3 and C1D4 patients were premedicated with 36 mg of dexamethasone according to the following the timepoints: 8 mg PO dexamethasone approximately 12 to 16 hours prior to administration of the maintenance dose of etentamig; 8 mg PO dexamethasone 2-5 hours (+/−1 hour) prior to administration of the maintenance dose of etentamig, and 20 mg IV dexamethasone 15 to 60 minutes prior to administration of the maintenance dose of etentamig. The maintenance dose of etentamig was delivered via IV infusion.

TABLE 18
Step-Up Dose (SUD) Experimental Design (Total Enrolled n = 70)

| Key Eligibility | | | | | Primary |
|---|---|---|---|---|---|
| Criteria: | Study Design | | | | Endpoint |
| ≥18 years | Part 1 | | Part 2 | | Incidence of |
| ≥3 prior lies of | A1: Dose level 1 (n = 26) | | A2: No expansion | | grade ≥2 CRS |
| therapy including | | | | | |
| exposure to PI, | 2 mg | 60 mg | | | |
| IMiD and CD38- | etentamig | etentamig | | | |
| AB | D1 (step- | D4 | | | |
| R/R to last therapy | up dose) | (maintenance | | | |
| No exposure to | | dose) | | | |
| BCMA-targeted | 10 mg | 10 mg | | | |
| therapy | DEX (15 | DEX (15 | | | |
| | to 60 | to 60 | | | |
| | minutes | minutes | | | |
| | before | before | | | |
| | step-up | maintenance | | | |
| | dose) | dose) | | | |
| | A2: Dose level 2 (n = 21) | | A1: Expansion (n = 23) | | |
| | 4 mg | 60 mg | 2 mg | 60 mg | |
| | etentamig | etentamig | etentamig | etentamig | |
| | D1 (step- | D4 | D1 (step- | D4 | |
| | up dose) | (maintenance | up dose) | (maintenance | |
| | | dose) | | dose) | |
| | 10 mg | 10 mg | 10 mg | 8 mg DEX | |
| | DEX (15 | DEX (15 | DEX (15 | (12-16 hours before | |
| | to 60 | to 60 | to 60 | maintenance dose) | |
| | minutes | minutes | minutes | 8 mg DEX (2-5 | |
| | before | before | before | hours ±1 hour | |
| | step-up | maintenance | step-up | before maintenance | |
| | dose) | dose) | dose) | dose) | |
| | | | | 20 mg DEX | |
| | | | | (15 to 60 | |
| | | | | minutes before | |
| | | | | maintenance | |
| | | | | dose) | |

Abbreviations: PI (Proteasome inhibitor); IMiD (Immunomodulatory imide); CD38-AB (anti-CD38 antibody); R/R = Relapsed or refractory; D1 = day 1; D4 = day 4.

As shown in Table 19, any grade CRS was reported in 10 (39%) and 11 (52%) patients during Part 1 at 2 mg and 4 mg step-up doses, respectively. Specifically, CRS grade ≥2 occurred in 3 (12%) of patients that received the 2 mg step-up dose (SUD) and in 6 (29%) of patients that received the 4 mg step-up dose, leading to selection of 2 mg as the step-up dose for Part 2.

TABLE 19
CRS Incident and Severity in Dose Escalation

| | 2 mg/60 mg | 4 mg/60 mg |
|---|---|---|
| | (ESC: Cohort A1) | (ESC: Cohort A2) |
| | N = 26 | N = 21 |
| Dexamethasone premedication | CID1: 10 mg | CID1: 10 mg |
| | CID4: 10 mg | CID4: 10 mg |
| Overall CRS, n (%) | 10 (39%) | 11 (52%) |
| Grade 1 CRS, n (%) | 7 (31%) | 5 (24%) |
| | After SUD: 1 (4%) | After SUD: 3 (14%) |
| | After Maintenance dose: | After Maintenance dose: |
| | 7 (27%) | 3 (14%) |
| Grade 2 CRS, n (%) | 3 (12%) | 5 (24%) |
| | After SUD: 0 (0%) | After SUD: 4 (19%) |
| | After Maintenance dose: | After Maintenance dose: |
| | 3 (12%) | 1 (5%) |
| Grade ≥ 3 CRS, n (%) | 0 | 1 (5%) |
| | After SUD: 0 | After SUD: 0 |
| | After Maintenance dose: 0 | After Maintenance dose: |
| | | 1 (5%) |

Specifically, in Part 1, a step-up dose of 2 mg etentamig IV on CID1 followed by the maintenance dose of 60 mg etentamig IV on C1D4 (Cohort A1) demonstrated a considerable reduction in the overall incidence and incidence of Grade ≥2 CRS compared to the step-up cohort with 4 mg (Cohort A2). Additionally, numerically higher incidence of overall (52% vs 39%), Grade 2 CRS (24% vs 12%), and Grade 3 CRS (5% vs 0%) was observed with 4 mg/60 mg cohort compared to 2 mg/60 mg cohort.

Table 20 illustrates a summary of all CRS events in cycle 1 (Part 1 and Part 2). In Part 2, (with a modified premedication dose regimen) 7 (30.4%) patients experienced CRS: 1 (4.3%) patient experienced grade 2 CRS, no patients experienced grade ≥3 CRS events, and only 2 (9%) patients received tocilizumab to treat CRS after the maintenance dose, and no patients received tocilizumab to treat CRS after the step-up dose. Overall, median time to CRS onset was 14.3 hours and median time to CRS resolution was 8.7 hours after the maintenance dose. No patients had recurrent CRS after cycle 1.

TABLE 20
Summary of CRS Events in Cycle 1

| | Part 1 | | | | Part 2 | | | |
|---|---|---|---|---|---|---|---|---|
| | Escalation | | Escalation | | Expansion | | | |
| | 2 mg/60 mg | | 4 mg/60 mg | | 2 mg/60 mg | | All Subjects | |
| | (N = 26) | | (N = 21) | | (N = 23) | | (N = 70) | |
| Summary of | Step-up | Maintenance | Step-up | Maintenance | Step-up | Maintenance | Step-up | Maintenance |
| CRS | Dose | Dose | Dose | Dose | Dose | Dose | Dose | Dose |
| Any CRS, n | 10 | | 11 | | 7 | | 28 | |
| (%) | (38.5) | | (52.4) | | (30.4) | | (40.0) | |
| Subjects with | 1 | | 1 | | 1 | | 3 | |
| recurrent | (3.8) | | (4.8) | | (5.3) | | (4.5) | |
| CRS in step- | | | | | | | | |
| up dose | | | | | | | | |
| interval and | | | | | | | | |
| maintenance | | | | | | | | |
| dose interval, | | | | | | | | |
| n (%) | | | | | | | | |
| Subjects with | 0 | 3 | 4 | 2 | 0 | 1 | 4 | 6 |
| Grade ≥2 | | (11.5) | (19.0) | (9.5) | | (4.3) | (5.7) | (8.6) |
| CRS, n (%) | | | | | | | | |
| Maximum | | | | | | | | |
| grade, n (%) | | | | | | | | |
| Grade 1 | 1 | 7 | 3 | 3 | 1 | 6 | 5 | 16 |
| | (3.8) | (26.9) | (14.3) | (14.3) | (4.3) | (26.1) | (7.1) | (22.9) |
| Grade 2 | 0 | 3 | 4 | 1 | 0 | 1 | 4 | 5 |
| | | (11.5) | (19.0) | (4.8) | | (4.3) | (5.7) | (7.1) |
| Grade 3 | 0 | 0 | 0 | 1 | 0 | 0 | 0 | 1 |
| | | | | (4.8) | | | | (1.4) |
| Grade 4 | 0 | 0 | 0 | 0 | 0 | 0 | 0 | 0 |
| Grade 5 | 0 | 0 | 0 | 0 | 0 | 0 | 0 | 0 |
| Median time | NA | 8.3 | 11.3 | 13.3 | NA | 22.9 | 17.9 | 14.3 |
| to onset^{a} | | (5.8, 25.0) | (8.5, 28.3) | (5.6, 39.4) | | (5.5, 29.6) | (8.5, 28.3) | (5.5, 39.4) |
| (range), | | | | | | | | |
| hours | | | | | | | | |
| Median time | NA | 9.3 | 9.7 | 6.2 | NA | 25.2 | 9.7 | 8.7 |
| to | | (3.8, 32.4) | (1.4, 40.3) | (2.9, 28.0) | | (1.8, 107.7) | (1.4, 40.3) | (1.8, 107.7) |
| resolution^{b}, | | | | | | | | |
| hours (range) | | | | | | | | |
| Tocilizumab | 1 | 3 | 5 | 2 | 0 | 2 | 6 | 7 |
| use for CRS | (3.8) | (11.5) | (23.8) | (9.5) | | (8.7) | (8.6) | (10.0) |
| management, | | | | | | | | |
| n (%) | | | | | | | | |

^{a}Time to onset of CRS is calculated by (Start Date/Time of CRS − Date/Time of Last Previous Dose).
^{b}Time to resolution of CRS is calculated by (End Date/Time of CRS − Start Date/Time of CRS). When two or more CRS events occurred in one dose interval for a given subject, the events were combined as one CRS event in calculation of time to resolution.

ICANS occurred in 7 (10%) patients; 3 (4%) experienced grade 3 ICANS events. Most common any-grade treatment-emergent adverse events (TEAEs) were CRS (40%, representing total CRS events across Parts 1 and 2), neutropenia (37%), diarrhea (29%), anemia (24%), and fatigue (20%). Most frequent grade 3/4 TEAEs were neutropenia (31%), anemia (19%), thrombocytopenia (13%), and leukopenia (11%). Grade 5 TEAEs occurred in 5 patients; 1 had a TEAE deemed as possibly related to etentamig (COVID-19 pneumonia).

In the efficacy-evaluable population shown in Table 21 (n=68), the objective response rate was 62% (95% CI: 49.2, 73.3); 35 (52%) patients achieved a VGPR or better. Median time to response was 1.1 mo (range: 1-4) and median duration of follow-up was 5.8 mo (range: 1-12).

TABLE 21
Efficacy Evaluation Summary of Response

| | All Subjects |
|---|---|
| Response | (N = 68) |
| Median duration of follow-up (months) (Min, Max) | 5.8 (1, 12) |
| ORR, n (%)^{a} | 42 (61.8) |
| 95% CI | 49.18, 73.29 |
| Best overall response, n (%)^{b} | |
| SCR | 6 (8.8) |
| CR | 3 (4.4) |
| VGPR | 26 (38.2) |
| PR | 7 (10.3) |
| Minimal response | 0 |
| Stable disease | 16 (23.5) |
| Progressive disease | 10 (14.7) |
| Median time to first objective response | 1.1(1, 4) |
| (months) (Min, Max)^{h} | |

^{a}ORR was calculated as the percentage of subjects with a confirmed stringent complete response, complete response, very good partial response, or partial response. The 2-sided 95% exact binomial CI of ORR was summarized using the Clopper-Pearson method.
^{b}Best overall response was determined by the Investigator using IMWG 2016 criteria.

An expanded patient population is summarized in Table 22 (n=70). Median duration to follow up was 7.2 months (range 1-14 months), and treatment was ongoing in 40 (57%) of patients at the time of reporting. The primary reason for treatment discontinuation was disease progression in 22 (31%) of patients. The median duration of etentamig was 6.7 months (range 1-15 months).

TABLE 22
Follow-up Characteristics

| | Escalation | Escalation | Expansion | |
|---|---|---|---|---|
| | 2 mg /60 mg | 4 mg/60 mg | 2 mg/60 mg | Total |
| | n = 26 | n = 21 | n = 23 | N = 70 |
| Median duration of follow-up, | 8.6 (1-14) | 9.7 (1-11) | 5.3 (1-7) | 7.2 (1-14) |
| months (range) | | | | |
| Treatment ongoing, n (%) | 13 (50) | 11 (52) | 16 (70) | 40 (57) |
| Primary reason for treatment | | | | |
| discontinuation, n (%) | | | | |
| Disease progression | 8 (31) | 9 (43) | 5 (22) | 22 (31) |
| Adverse events | 3 (12) | 0 | 1 (4) | 4 (6) |
| Withdrawal | 1 (4) | 0 | 0 | 1 (1) |
| Other | 1 (4) | 1 (5) | 1 (4) | 3 (4) |
| Median duration of etentamig, | 9.1 (1-15) | 9.5 (1-12) | 4.9 (1-7) | 6.7 (1-15) |
| months (range) | | | | |
| Median relative dose | 100 (93-150) | 100 (83-117) | 100 (80-200) | 100 (80-200) |
| intensity^{a} of | | | | |
| etentamig, % (range) | | | | |

^{a}relative dose intensity is calculated as total actual dose (mg) divided by (60 mg × Int [date of last dose − date of first dose + 28]/28) × 100

As shown in Table 23, the expanded patient population (n=70) demonstrated etentamig with SUD and a modified premedication dose regimen resulted in a manageable safety profile in patients with RRMM. Immune effector cell-associated neurotoxicity syndrome was reported in 7 (10%) patients (grade 1: n=2 [3%]; grade 2: n=2 [3%]; grade 3: n=3 [4%]). Median time to onset was 17.1 (range 4.9-68.1) hours and median time to resolution was 24.0 (range 7.8-61.2) hours. Six treatment-emergent adverse events (TEAE) leading to death occurred during the study. Two deaths were due to sepsis, two were due to disease progression, and one was due to unknown reason. None of these events were deemed related to etentamig. One death due to worsening of COVID-19 pneumonia was deemed possibly related to etentamig.

TABLE 23
Treatment-Emergent Adverse Events (TEAE) Characteristics

Total (n = 70)

| Characteristic | Any Grade | Grade 3-4 |
|---|---|---|
| Any TEAE | 69 (99) | 51 (73) |
| Hematologic^{a} | | |
| Neutropenia | 37 (53) | 31 (44) |
| Anemia | 19 (27) | 14 (20) |
| Thrombocytopenia | 12 (17) | 9 (13) |
| Nonhematologic^{a} | | |
| CRS | 28 (40) | 1 (1) |
| Diarrhea | 22 (31) | 1 (1) |
| Pneumonia | 17 (24) | 12 (17) |
| Fatigue | 16 (23) | 0 |
| Cough | 14 (20) | 0 |
| Upper respiratory infection | 11 (16) | 1 (1) |
| TEAE leading to: | | |

| | |
|---|---|
| Etentamig discontinuation | 3 (4) |
| Etentamig interruption | 36 (51) |
| Serious AEs | 36 (51) |

^{a}Cutoff: any grade frequence of ≥15%; CRS, cytokine reselse syndrome; TEAE, treatment-emergent adverse event.

Comparison between different dosing regimens of etentamig indicated that including a single step-up dose of etentamig and a modified premedication dose regimen of dexamethasone (DEX) prior to the maintenance dose of etentamig reduced the frequency and severity of CRS. Briefly, the following cohorts were compared: (1) 60 mg etentamig Q3W with 10 mg DEX premedication (“60 mg Q3W”); (2) 60 mg etentamig Q4W with 36 mg total DEX premedication (8 mg DEX 12-16 hours before maintenance dose, 8 mg DEX 3-4 hours+1 hour before maintenance dose, and 20 mg DEX 15 to 60 minutes before maintenance dose) (“60 mg Q4W+Dex”); (3) 2 mg etentamig step-up dose with 10 mg DEX premedication, and 60 mg etentamig Q4W maintenance dose with 10 mg DEX premedication (“2 mg/60 mg Q4W+20 mg Dex”); and (4) 2 mg etentamig step-up dose with 10 mg DEX premedication, and 60 mg etentamig maintenance dose with 36 mg DEX premedication (8 mg DEX 12-16 hours before maintenance dose, 8 mg DEX 3-4 hours+1 hour before maintenance dose, and 20 mg DEX 15 to 60 minutes before maintenance dose) (“2 mg/60 mg Q4W+46 mg Dex”). A comparison of the four treatment cohorts indicates that cohorts with one step-up dose (Cohorts 3 and 4) in combination with DEX expansion resulted in lower instances of CRS in patients. CRS severity was graded according to the American Society for Transplantation and Cellular Therapy (ASTCT) 2019 guidelines.

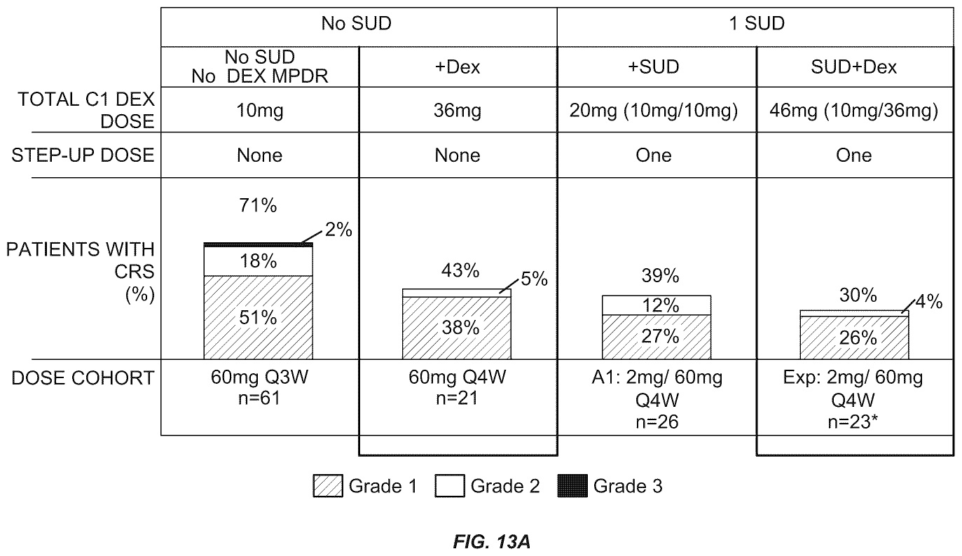

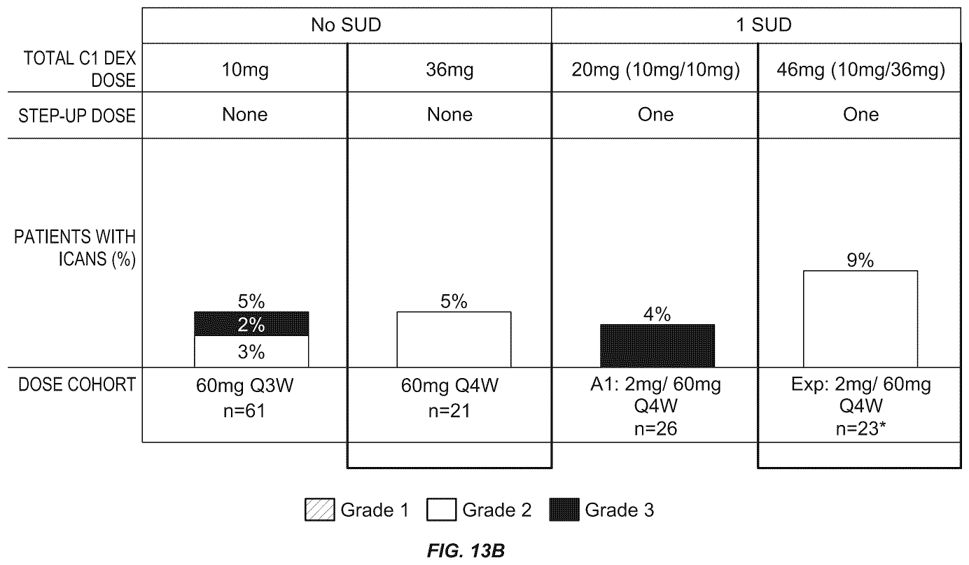

For the 60 mg Q3W cohort (n=61), 71% of patients exhibited CRS symptoms (FIG. 13A, leftmost column). Of the 71% experiencing CRS, 2% were classified as Grade 3, 18% as Grade 2, and the remaining 51% as Grade 1. For the 60 mg Q4W+Dex cohort (n=21), 43% of patients exhibited CRS symptoms (FIG. 13A, left center column). Of the 43% of patients experiencing CRS, 5% were classified as Grade 2 and 28% were classified as Grade 1. In contrast, for the 2 mg/60 mg Q4W+20 mg DEX cohort (n=26), 39% of patients experienced CRS symptoms (FIG. 13A, right center column). Of the 39% of patients experiencing CRS, 12% were classified as Grade 2 and 27% as Grade 1. Finally, for the 2 mg/60 mg Q4W+46 mg DEX cohort (n=23*), 30% of patients exhibited CRS (FIG. 13A, rightmost column) [*The three subjects received 8 mg DEX 12-16 hours before maintenance dose, 8 mg DEX 3-4 hours+1 hour before maintenance dose, and 10 mg DEX 15 to 60 minutes before maintenance dose]. Of the 30% of patients experiencing CRS, 4% were classified as Grade 2 and 26% as Grade 1.

Comparison of the overall instances of ICANS for each of the cohorts indicated that they were similar across treatment conditions. ICANS severity was graded according to the American Society for Transplantation and Cellular Therapy (ASTCT) 2019 guidelines. The results indicated that 5% of patients in the 60 mg Q3W cohort exhibited ICANS symptoms (FIG. 13B, leftmost column). Of the 5% experiencing ICANS, 2% were classified as Grade 3 and 3% as Grade 2. Similarly, 5% of patients in the 60 mg Q4W cohort exhibited ICANS symptoms, all of which were Grade 2 (FIG. 13B, left center column). For the 2 mg/60 mg Q4W+20 mg DEX cohort, 4% of patients experienced ICANS symptoms, all of which were Grade 3 (FIG. 13B, right center column). For the 2 mg/60 mg Q4W+46 mg DEX cohort, 9% of patients exhibited ICANS, all of which were Grade 2 (FIG. 13B, rightmost column).

Further, response data indicates that adding a SUD before a maintenance dose, in combination with DEX, does not compromise the overall effectiveness of etentamig as measured by overall response rate (ORR) and VGPR percent (Table 24).

TABLE 24
Higher dose DEX and Addition of SUD Does
Not Compromise Efficacy of Etentamig

| | NO SUD | | Addition of SUD |
|---|---|---|---|
| | 60 mg Q3W | 60 mg Q4W | 2 mg/60 mg |
| | (n = 61) | (n = 21) | (n = 26) |
| Total C1 Dex volume | 10 mg | 36 mg | 20 mg |
| | | | (10 mg/10 mg) |
| Step-up dose | None | None | One |
| Median duration F/U | 24.2 | 4.1 | ~3-5 months |
| (months) | (0.6-33.4) | (0.8-5.2) | |
| ORR (%) | 60% | 65% | 62% |
| ≥VGPR (%) | 52% | 55% | 58% |

The expanded cohort (n=70) confirmed that higher dose DEX and the addition of SUD does not compromise the efficacy of etentamig. As shown in Table 25 total ORR was 69% with 56% of patients experiencing a >VGPR.

TABLE 25
Overall Response Rate is Similar Across Cohorts that include SUD

| | Escalation | Escalation 4 | Expansion 2 | |
|---|---|---|---|---|
| | 2 mg /60 mg | mg/60 mg | mg/60 mg | Total |
| | n = 26 | n = 21 | n = 23 | N = 70 |
| ORR (%) | 73% | 67% | 65% | 69% |
| ≥VGPR (%) | 73% | 53% | 39% | 56% |
| SCR/CR | 27% | 24% | 9% | 20% |
| VGPR | 46% | 29% | 30% | 36% |
| PR | 0% | 14% | 26% | 13% |
| Responders | n = 19 | n = 14 | n = 15 | n = 48 |
| Median time to | 8 (6-25) | 1.1 (1-4) | 1.2 (1-3) | 1.1 (1-4) |
| first response, | | | | |
| months^{a} (range) | | | | |

^{a}The time to objective response is defined as the time of first dose of etentamig to first response of the confirmed objective response (sCR, CR, VGPR, or PR). CR, complete response; ORR, overall response rate, PR, partial response; sCR stringent complete response; VGPR, very good partial response.

Taken together, the clinical data indicate that a step-up dosing regimen involving a step-up dose of etentamig before a maintenance dose of etentamig in combination with DEX premedication before each dose of etentamig is able to further reduce frequency and severity of CRS in patients with RRMM, without increasing the instances of ICANS or decreasing the efficacy of etentamig.

### 6.8 EXAMPLE 8-Addition of SUD in combination with Modified Premedication Dose Regimen (MPDR) reduces serum indicators of adverse events associated with CRS

This Example demonstrates that 2 mg step-up dose etentamig+60 mg maintenance dose of etentamig in combination with a modified premedication dose regimen results in lower instances of serum indicators of adverse events related to CRS, as compared to etentamig without a SUD or a SUD without MPDR.

Briefly, four cohorts were treated:
(1) 60 mg etentamig Q3W without a step-up dose of etentamig and without MPDR (“60 mg Q3W”);
(2) 60 mg etentamig Q4W with a MPDR (36 mg total DEX) and without a step-up dose (“60 mg Q4W+MPD”);
(3) 2 mg etentamig step-up dose with 10 mg DEX premedication and 60 mg etentamig maintenance dose without MPDR (only 10 mg DEX premedication administered prior to the 60 mg of etentamig; “2 mg/60 mg Q4W+20 mg Dex”); and
(4) 2 mg etentamig step-up dose with 10 mg DEX premedication and 60 mg etentamig maintenance dose with MPDR (36 mg total DEX premedication administered prior to the 60 mg etentamig; “2 mg/60 mg Q4W+46 mg DEX”).
   All pharmacokinetic measurements were taken from peripheral blood and serum samples from patients in each cohort and were collected at baseline (post-dexamethasone, pre-etentamig), on treatment, and at disease progression.

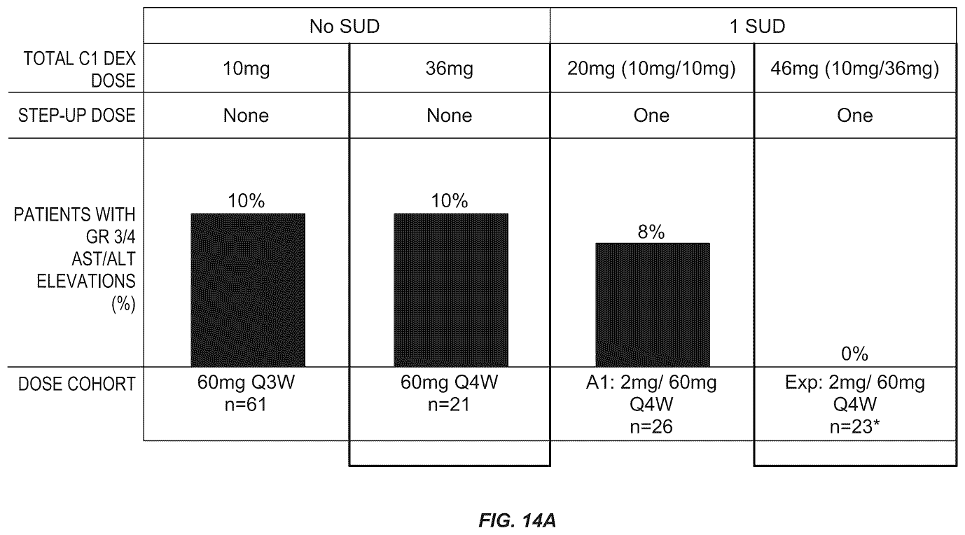

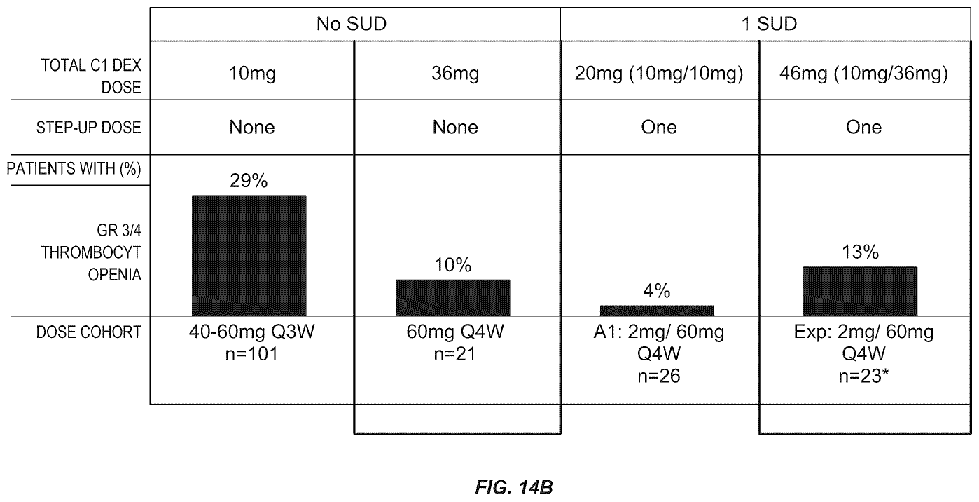

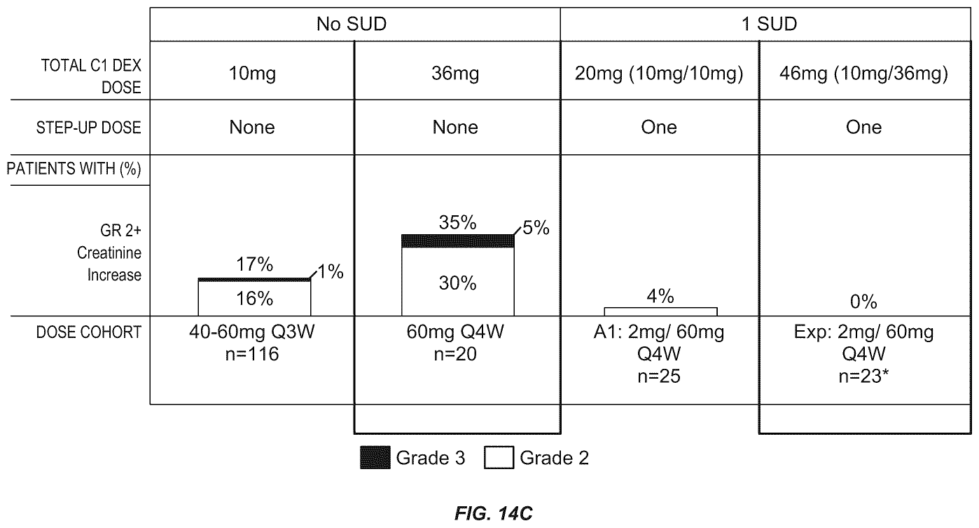

The results demonstrated that 2 mg step-up dose etentamig+60 mg maintenance dose of etentamig in combination with MPDR resulted in lower instances of liver abnormalities as measured by percentage of patients with grade 3/4 AST/ALT elevation. Specifically, 10% of patients in both cohorts without a SUD experienced grade 3/4 AST/ALT elevation, independent of total cycle 1 DEX (FIG. 14A, leftmost column compared to left center column; n=61 and n=21, respectively). In contrast, 8% of the patients in the 2 mg/60 mg Q4W+20 mg DEX cohort exhibited grade 3/4 elevated AST/ALT levels (FIG. 14A center column). Among the 2 mg/60 mg Q4W+46 mg DEX cohort, none exhibited grade 3/4 AST/ALT elevation (FIG. 14A, rightmost column).

Grade 3/4 thrombocytopenia was also lower for the 2 mg/60 mg Q4W+20 mg DEX cohort compared to other treatment conditions. Specifically, 29% of patients receiving either 40 or 60 mg etentamig combined with 10 mg DEX on a Q3W dosing regimen (n=101) experienced grade 3/4 thrombocytopenia (FIG. 14B, leftmost column). The incidence of grade 3/4 thrombocytopenia was 10% for the 60 mg Q4W+Dex cohort (FIG. 14B, left middle column). The incidence of grade 3/4 thrombocytopenia was decreased to 4% in the 2 mg/60 mg Q4W+20 mg DEX cohort (FIG. 14B, right middle column). Finally, 13% of patients in the 2 mg/60 mg Q4W+46 mg DEX cohort experienced grade 3/4 thrombocytopenia (FIG. 14B, rightmost column).

2 mg step-up dose etentamig+60 mg maintenance dose of etentamig in combination with MPDR resulted in lower instances of grade 2+creatinine increase compared to cohorts without SUD. 17% of patients receiving either 40 or 60 mg etentamig combined with 10 mg DEX on a Q3W dosing regimen (n=116) experienced grade 2+creatinine increase, 1% of which was grade 3 (FIG. 14C, leftmost column). 35% of patients in the 60 mg Q4W+DEX cohort experienced grade 2+creatinine increase, 5% of which was grade 3 (FIG. 14C, left middle column). 4% of patients in the 2 mg/60 mg Q4W+20 mg DEX cohort experienced grade 2 creatinine increase (FIG. 14B, right middle column). Finally, no patients in the 2 mg/60 mg Q4W+46 mg DEX cohort experienced grade 2+creatinine increase (FIG. 14B, rightmost column).

Taken together, these results indicate that 2 mg step-up dose etentamig+60 mg maintenance dose of etentamig in combination with MPDR results in lower instances of serum indicators of adverse events related to CRS, relative to etentamig without a SUD or a SUD without MPDR.

### 6.9 EXAMPLE 9-Reduced Peak Levels of Pro-inflammatory Cytokines Support Lower Overall CRS Rates Observed with Addition of SUD

This example demonstrates that the lower overall CRS rates observed with cohorts that include SUD are supported by reduced peak levels of pro-inflammatory cytokines.

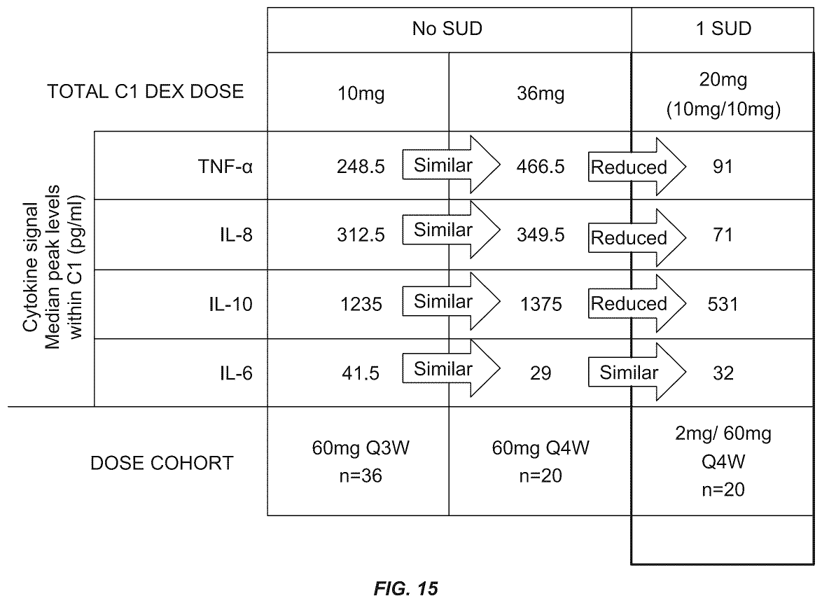

Briefly, cytokine signal median peak levels (pg/ml) were measured within cycle 1 for three cohorts: (1) 60 mg etentamig Q3W with 10 mg DEX premedication and without a step-up dose (“60 mg Q3W”) (n=36; FIG. 15, left column); (2) 60 mg etentamig Q4W with a MPDR (36 mg total DEX) and without a step-up dose (“60 mg Q4W+DEX”) (n=20; FIG. 15, middle column); and (3) 2 mg etentamig step-up dose with 10 mg DEX premedication and 60 mg etentamig maintenance dose without MPDR (only 10 mg DEX premedication administered prior to the 60 mg etentamig maintenance dose; “2 mg/60 mg Q4W+20 mg DEX”) (n=20; FIG. 15, right column). Peak cytokine levels of TNF-α, IL-8, IL-10, and IL-6 were not impacted by increased total DEX administration. In contrast, the addition of SUD resulted in steep reduction (60-80%) in peak levels of IL-8, IL-10, and TNF-α.

Evaluation of peak pro-inflammatory cytokine levels within the cohorts with step-up dosing of etentamig indicated that reduction in peak levels of CRS-related cytokines was most pronounced in the 2 mg/60 mg Q4W+20 mg DEX Expansion cohort. Table 26 illustrates peak cytokine levels across three different cohorts that include SUD.

TABLE 26
Reduction in CRS-related Cytokines is Largest in 2 mg/60 mg Expansion Cohort

| | | 60 mg Q4W | Escalation | Escalation 4 | Expansion 2 |
|---|---|---|---|---|---|
| | | Without SUD | 2 mg /60 mg | mg/60 mg | mg/60 mg |
| | | n = 20 | n = 26 | n = 21 | n = 22 |
| Peak Levels | IL-6 | 29.0 | 17.0 | 16.0 | 10.2 |
| (median | IL-8 | 349.5 | 71.0 | 114.0 | 52.0 |
| pg/mL) | TNF-α | 466.5 | 91.0 | 162.0 | 61.0 |

CRS, cytokine release syndrome;
IL, interleukin;
TNR, tumor necrosis factor

Taken together the clinical data indicates that peak levels of pro-inflammatory cytokines (e.g., IL-8, IL-10 and TNF-α) support reduction in all grades of CRS with SUD.

### 6.10 EXAMPLE 10-Correlative Biomarker Analysis Indicates SUD Results in Lower Cmax Levels of Pro-inflammatory Cytokines and Biphasic Release Pattern

This Example demonstrates that incorporating a SUD of etentamig in the regimen results in lower concentrations and biphasic release pattern of pro-inflammatory cytokine markers associated with CRS, including IL-8, IL-10, and TNF-α.

Peripheral blood and serum samples from patients with RRMM who received etentamig at 20 mg Q3W, 40 mg Q3W, 60 mg Q3W, 60 mg Q4W, or 60 mg Q4W following a 2 mg step-up dose (SUD cohort) were collected at baseline (post-10 mg or 36 mg dexamethasone, pre-etentamig), on treatment, and at disease progression. Samples were analyzed by Luminex for cytokines.

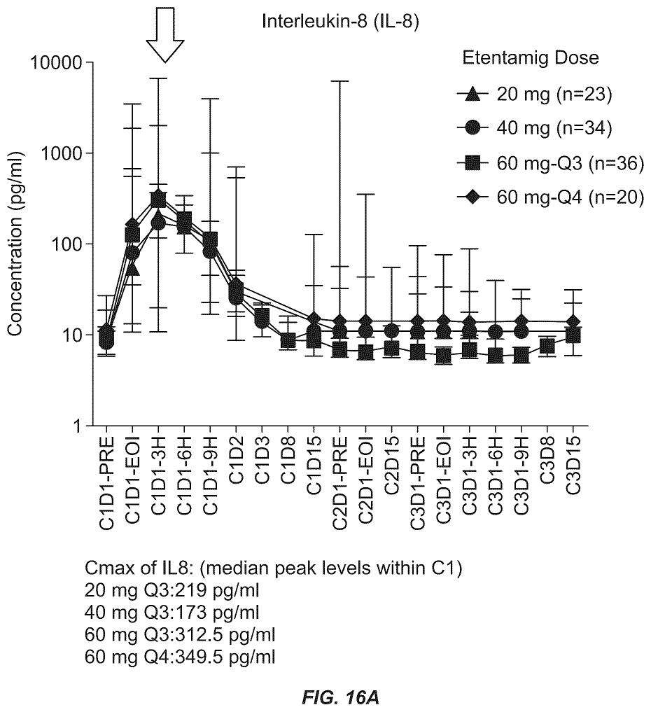

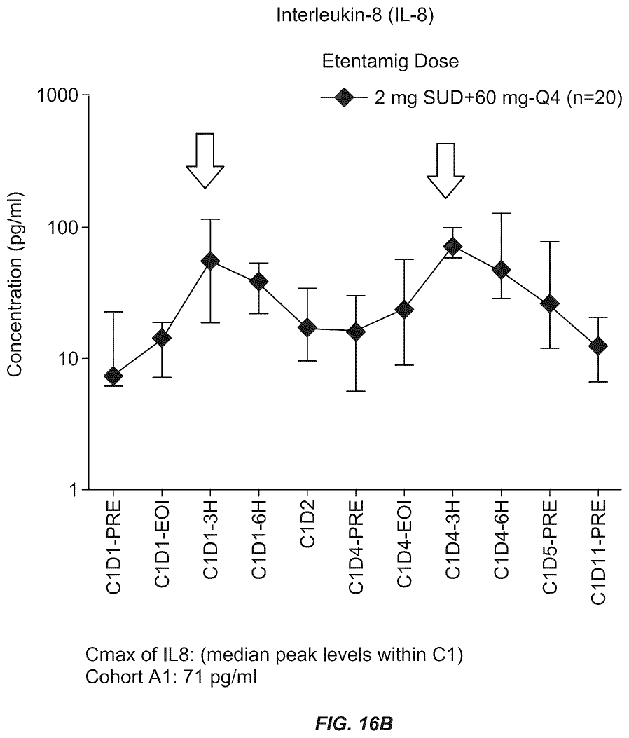

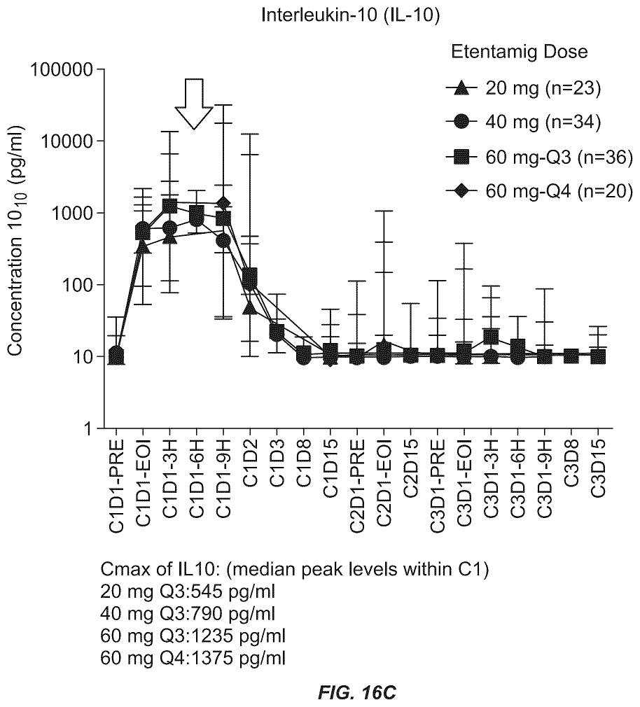

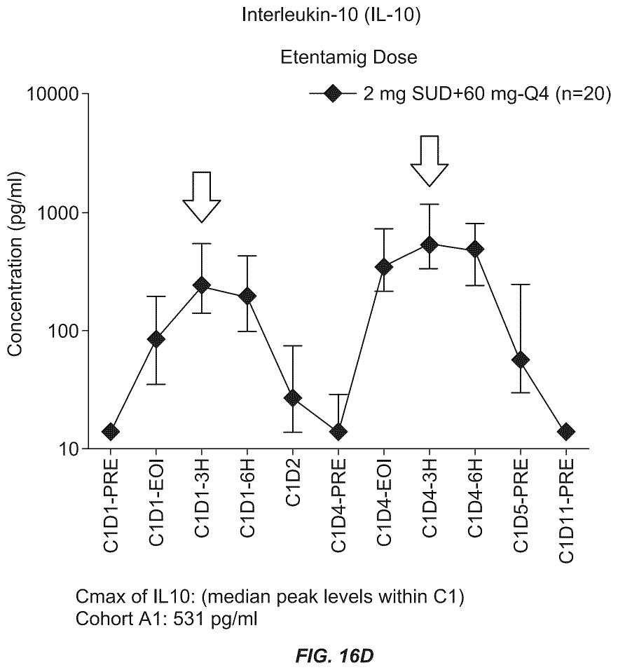

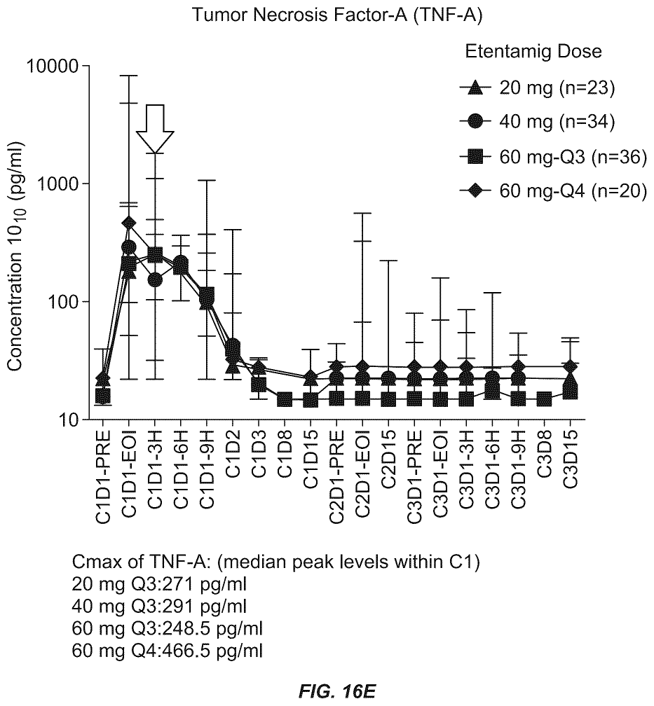

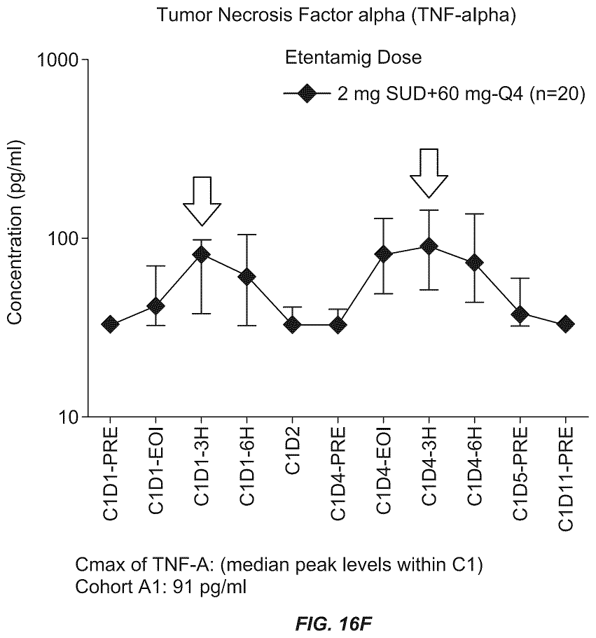

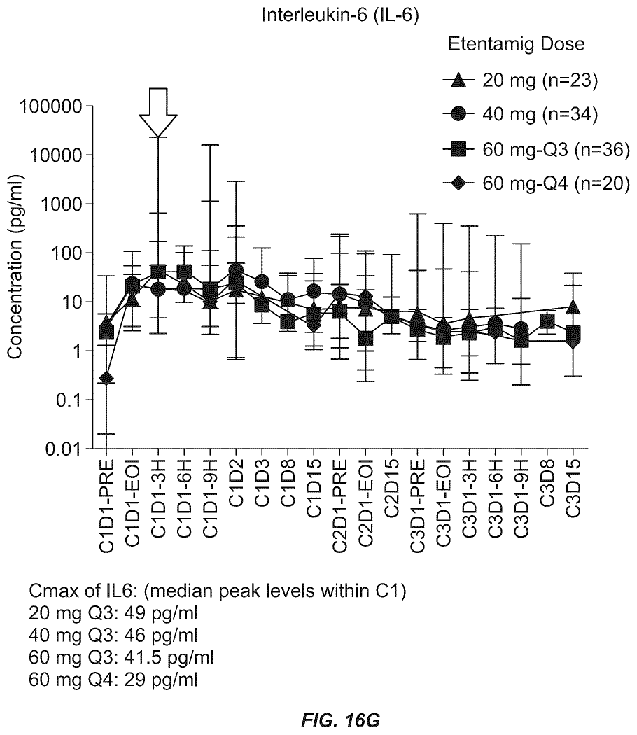

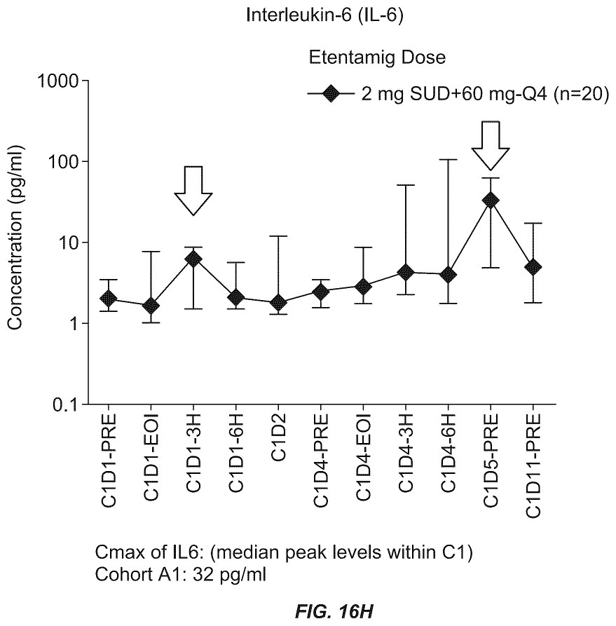

Having a 2 mg step-up dose of etentamig followed by 60 mg Q4W resulted in lower Cmax concentrations of pro-inflammatory cytokine markers IL-8, IL-10, and TNF-α than all other treatment cohorts (FIG. 16A-FIG. 16H). Following treatment with 60 mg Q4W of etentamig, proinflammatory cytokine maximum serum concentrations within cycle 1 peaked at 349.5 pg/mL for IL8 (FIG. 16A), and at 71 μg/ml with a 2 mg step-up dose of etentamig (FIG. 16B). Likewise treatment with 60 mg Q4W of etentamig resulted in maximum serum concentrations within cycle 1 of 1375 μg/mL for IL-10 (FIG. 16C), and 531 μg/ml with a 2 mg step-up dose of etentamig (FIG. 16D). Similarly, TNF-α serum concentrations peaked at 466.5 pg/ml following 60 mg Q4W of etentamig (FIG. 16E) and just 91 μg/ml with a 2 mg step-up dose of etentamig (FIG. 16F). In contrast, a 2 mg step-up dose of etentamig did not result in a reduction in maximum serum concentrations of IL-6, with maximum serum concentration peaking at 29 μg/mL following 60 mg Q4W of etentamig (FIG. 16G) and 32 μg/ml with SUD (FIG. 16H).

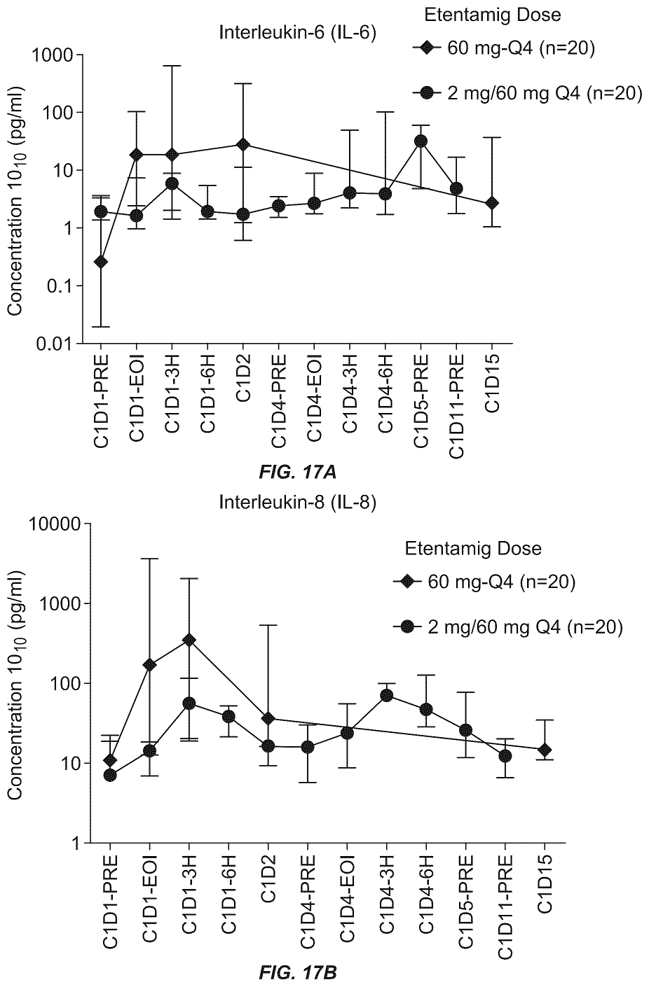

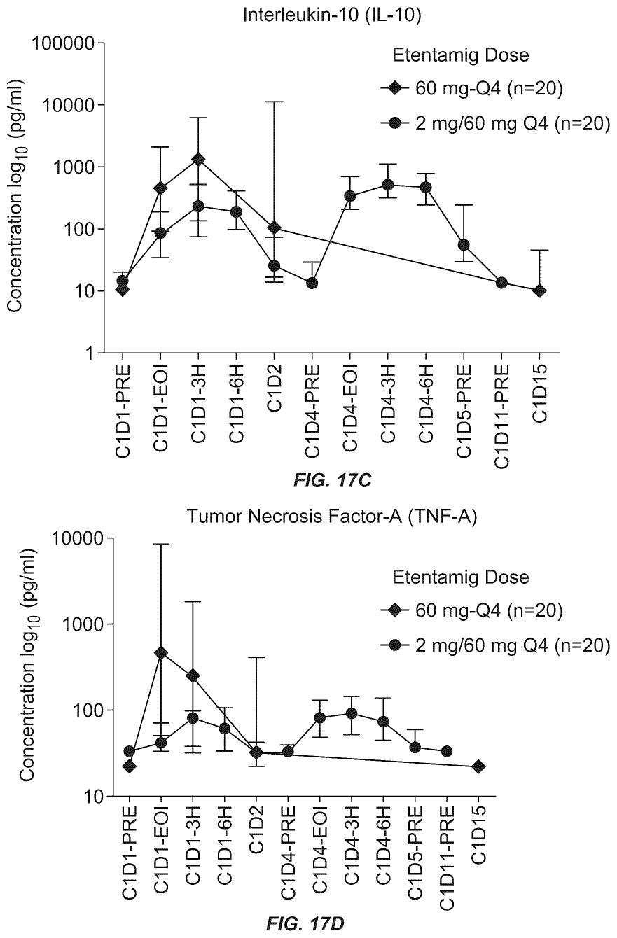

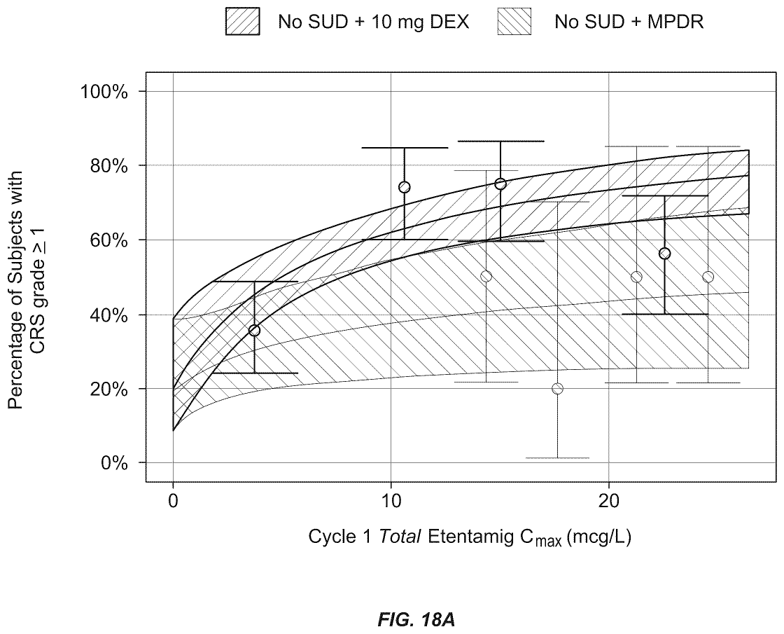

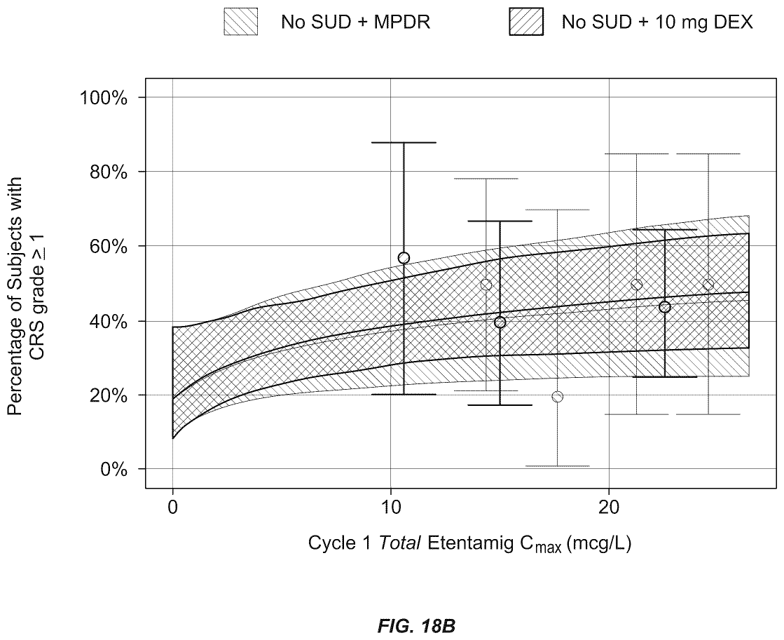

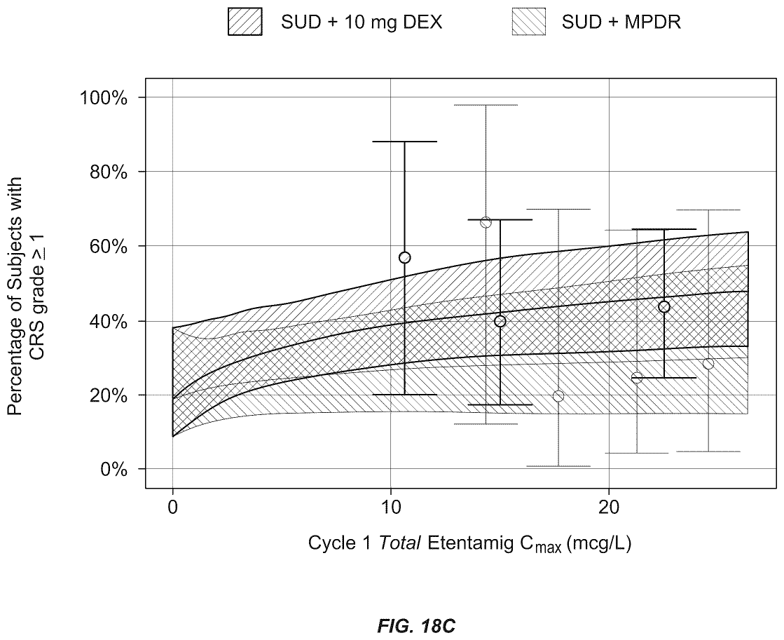

A 2 mg step-up dose of etentamig also resulted in a delayed peak and biphasic increase in pro-inflammatory cytokines in comparison to 60 mg Q4W release. As shown in FIGS. 17A-17D, administering both the step-up dose and maintenance dose of etentamig enhanced the production of IL-6 (FIG. 17A), IL-8 (FIG. 17B), IL-10 (FIG. 17C) and TNF-α cytokines (FIG. 17D). However, collectively, peak cytokine levels were observed at day 4 in the SUD cohort compared to day 1 in the 60 mg Q4W release cohort (FIGS. 17A-17D).

In sum, this example demonstrates that including a 2 mg step-up dose of etentamig in the dosing regimen results in lower concentrations and biphasic release pattern of pro-inflammatory cytokine markers associated with CRS, including IL-8, IL-10, and TNF-α.

### 6.11 EXAMPLE 11-Peak Activation/Proliferation of CD8 T Cells is Comparable in SUD and 60 mg Q4W Treated Patients

This example demonstrates that peak activation and proliferation of CD8 T-cells is comparable across the three SUD cohorts and patients treated with etentamig Q4W. As shown in Table 27 CD8+ T-cell activation and proliferation were measured by fold change in CD69+ and Ki67+expression levels (respectively). Fold change was comparable across SUD cohorts and Q4W treated patients.

TABLE 27
CD8+ T Cell Activation and Proliferation
is Comparable Across SUD and Q4W Cohorts

| | | 60 mg Q4W | Escalation | Escalation 4 | Expansion 2 |
|---|---|---|---|---|---|
| | | without SUD | 2 mg /60 mg | mg/60 mg | mg/60 mg |
| | | n = 20 | n = 26 | n = 21 | n = 22 |
| CD8+ T Cells | CD69+ | 1.9 | 1.9 | 1.6 | 1.9 |
| (median FC) | Ki67+ | 2.0 | 3.6 | 3.8 | 2.2 |

FC, Fold change;
Q4W, once every 4 weeks;
SUD, step-up dose

In sum, this example demonstrates that expansion and proliferation of CD8+ T cells, as measured by fold change of CD69+ and Ki67+was not affected by differences between SUD treatment cohorts and patients treated with etentamig Q4W.

### 6.12 EXAMPLE 12—The Dosing Regimen of Etentamig is Safer and More Convenient Than Other CD3×BCMA Bispecific Antibodies

Comparison of the convenience and safety profile of the etentamig dosing regimen relative to other CD3×BCMA bispecific antibodies (e.g., teclistamab, elranatamab and linvoseltamab) was performed. As shown in Table 28, in contrast to teclistamab, elranatamab and linvoseltamab, etentamig only requires one step-up dose and can be administered Q4W. Moreover, the incidence of CRS for the etentamig dosing regimen is only 30% (26% Grade 1, 4% Grade 2, and 0% Grade 3), whereas the incidence of CRS was 72% (50% Grade 1, 21% Grade 2, and 1% Grade 3) for teclistamab; 59% (44% Grade 1, 14% Grade 2, and 1% Grade 3) for elranatamab; and 45% (35% Grade 1, 9% Grade 2, and 1% Grade 3) for linvoseltamab.

TABLE 28
Comparison of Currently available RRMM Treatment Options

| | Etentamig | Etentamig | | |
|---|---|---|---|---|
| | Expansion | 60 mg Q4W Dose | | |
| | 2 mg/60 mg | (N = 21) from | | |
| | (N = 23) from | Study TNB383B.0001 | | |
| | Study M24-108 | (+high Dex, | | |
| | (SUD + high Dex) | without SUD) | Teclistamab | Elranatamab |
| Regimen | Q4W | Q4W | Q1W | Q1W (may |
| | | | | extend to Q2W |
| | | | | after 24 weeks |
| | | | | per response) |
| Route of | IV | IV | SC | SC |
| Administration | | | | |
| SUD | Day 1: 2 mg | None | Day 1: SUD 1 | Day 1: SUD 1 |
| | Day 4: | (Day 1: | 0.06 mg/kg | 12 mg |
| | Maintenance | Maintenance | Day 4: SUD 2 | Day 4: SUD 2 |
| | dose | dose 60 mg) | 0.3 mg/kg | 32 mg |
| | | | Day 7: | Day 8: |
| | | | Maintenance | Maintenance |
| | | | dose 1.5 mg/kg | dose 76 mg |
| Dexamethasone | Total 46 mg | Total 36 mg | Total 48 mg | Total 60 mg |
| Premedication for | | | | |
| SUD and | 10 mg prior to | (8 mg PO 12 hours | (16 mg prior to | (20 mg prior to |
| Maintenance dose | SUD; 36 mg | predose; 8 mg PO/IV | each dose | each dose |
| in Cycle 1 | prior to | 3-4 hours predose; | including step- | including step- |
| | Maintenance | 20 mg IV/PO | up dose) | up dose) |
| | dose | 15-60 minutes | | |
| | | predose) | | |
| Rate of CRS | Overall | Overall incidence: | Overall | Overall |
| | incidence: 30% | 43% | incidence: 72% | incidence: 58% |
| | Grade 1: 26% | Grade 1: 38% | Grade 1: 50% | Grade 1: 44% |
| | Grade 2: 4% | Grade 2: 5% | Grade 2: 21% | Grade 2: 14% |
| | Grade 3: 0% | Grade 3: 0% | Grade 3: 1% | Grade 3: 1% |

Therefore, etentamig's dosing regimen offers improved safety and convenience relative to other CD3×BCMA bispecific T-cell redirecting antibodies.

### 6.13 EXAMPLE 13-Exposure-Response (ER) and Pharmacokinetic/Pharmacodynamic (PK/PD) Analyses for Etentamig's Dosing Regimen

This Example demonstrates PK/PD analyses for a 2 mg step-up dose with 60 mg maintenance dose Q4W and a MPDR (comprising 36 mg total dexamethasone administered prior to the maintenance dose).

Briefly, exposure-response (ER)-efficacy (N=286) and-safety (N=290) analyses were conducted using logistic regression methodology combining data from escalation (0.025-120 mg Q3W) and expansion (20, 40, 60 mg etentamig Q3W and 60 mg etentamig Q4W) cohorts of a phase 1 study involving an Arm A of a separate phase 1b study evaluating step-up dose escalation (2 or 4 mg on day 1) with a maintenance dose (60 mg on day 4) in patients with RRMM. In two of the cohorts, patients received a modified premedication dose regimen (comprising 36 mg total dexamethasone administered prior to the maintenance dose of 60 mg etentamig Q4W): (1) before receiving either 60 mg etentamig Q4W, or (2) before 60 mg etentamig Q4W that was preceded with a 2 mg etentamig step-up dose. In all other cohorts with or without 2 mg etentamig, patients received only 10 mg DEX. Safety endpoints (CRS: any-grade and ≥G2) were modeled with cycle 1 total (unbound+bound to soluble BCMA) Cmax, while ORR was modeled with free (unbound+partially bound to soluble BCMA) Cavg (logistic regression; R version 4.3.2). CRS ER models included a priori selected DEX (low vs. high) and SUD (yes vs. no) as predictors. Relevant patient-specific covariates were tested in all models.

CRS PK/PD analyses included total PK, IL-6, and CRS data (N=199) from the first 3 cycles of the above phase 1/1b studies. A semi-mechanistic indirect response PK/IL-6 model was developed to correlate total PK and IL-6 (NONMEM 7.5). Maximal IL-6 concentrations were derived using post hoc estimates from the PK/IL-6 model and correlated with CRS events (logistic regression; R version 4.3.2). The models included effects of time-dependent DEX (unmodified vs. modified) and SUD (yes vs. no). The overall modeling approach was assessed by comparing observed and predicted CRS rates. Simulations were conducted for various dosing regimens, including different one or two SUD and dexamethasone premedication scenarios to support selection of the optimal SUD and dexamethasone levels, according to the dosing regimens described below in Table 29.

TABLE 29
CRS PK/PD Modeling

| | Median of |
|---|---|
| | Simulated |
| | Peak IL-6 |
| | (5%, 95%), |
| Dosing Regimen | pg/mL |
| 60 mg etentamig + 10 mg DEX | 40.0 |
| | (9.42, 51800) |
| 60 mg etentamig + MPDR | 35.2 |
| | (8.52, 51800) |
| 2 mg SUD etentamig/60 mg etentamig + 10 mg DEX | 12.7 |
| | (2.56, 11800) |
| 2 mg SUD etentamig/60 mg etentamig + MPDR | 11.3 |
| | (1.96, 11800) |
| 2 mg SUD etentamig/2 mg SUD2 etentamig/60 mg | 8.90 |
| etentamig + 10 mg DEX | (1.46, 3760) |
| 2 mg SUD etentamig/3 mg SUD2 etentamig/60 mg | 8.64 |
| etentamig + 10 mg DEX | (1.40, 3390) |
| 2 mg SUD etentamig/4 mg SUD2 etentamig/60 mg | 8.46 |
| etentamig + 10 mg DEX | (1.37, 3100) |

Consistent with the observations described in the Examples above, any-grade CRS model predicted probabilities suggested two key outcomes. Table 30 shows these outcomes in a summary of exposure safety predictions for probability and observed any grade CRS events at various dosing regimens.

TABLE 30
Summary of Exposure-Safety (Any-grade CRS) Predictions

| | | | Etentamig | | |
|---|---|---|---|---|---|
| | | | PK | % Probability | |
| | Step-up | Dexamethasone | Median | of Any-grade | Observed % |
| | Dose | Premedication | Cₘₐₓ | CRS, Median | Any-grade |
| Dosing Regimen | (SUD) | Regimen | (μg/mL) | (95% CI) | CRS |
| 60 mg Q3W | No | Unmodified | 15.3 | 68.4 | 70 |
| | | | | (59.8, 76.4) | |
| 60 mg Q4W | No | Modified | 17.2 | 42.0 | 43 |
| | | | | (23.2, 62.3) | |
| 2 mg/60 mg Q4W | Yes | Unmodified | 17.3 | 43.3 | 39 |
| | | | | (29.9, 59.0) | |
| 2 mg/60 mg Q4W | Yes | Modified | 19.6 | 29.3 | 30 |
| | | | | (14.5, 50.8) | |

First, premedication with modified DEX (36 mg) and without a 2 mg step-up dose improves the CRS safety profile compared to the unmodified DEX (10 mg) also without a 2 mg step-up dose (42% vs 68%, respectively). Second, a modified premedication dosing regimen (36 mg) with 2 mg SUD further improves the CRS safety profile of etentamig (29% vs 43%, r). No ER relationship was observed for ≥G2 CRS (p >0.05). The ORR model resulted in a predicted probability of 68% ORR (60.5, 74.8, 95% confidence interval-closely matching with overall observed 64% ORR) with no impact of SUD and MPDR. Only sBCMA was selected as covariate in the ORR ER model.

The final semi-mechanistic PK/IL-6 model captured the observed IL-6 data, and the CRS rates (predicted by the logistic regression models) closely matched with the observed data. The CRS PK/PD analyses findings were consistent with the clinical observations and ER analyses key outcomes described above. The CRS PK/PD analyses comparing the 1-SUD vs 2-SUD (modeled) regimens with MPDR suggested that among the 2-SUD regimens with MPDR dexamethasone, the 2 mg step-up dose/3 mg step-up dose/60 mg maintenance dose regimen was predicted to have the maximum reduction of 4% points in any-grade CRS compared to the 2 mg step-up dose/60 mg maintenance dose 1-SUD regimen, however, with substantial overlap of confidence intervals and with no decrease in ≥G2 CRS (<0.5%), as shown in Table 31.

TABLE 31
Summary of CRS PK/PD Predictions - 1-SUD vs 2-SUD

| | Median of | | |
|---|---|---|---|
| | Simulated Peak | | |
| | IL-6 (5%, 95%), | Predicted % Probability (95% CI) | |
| Dosing Regimen | pg/mL | Any -grade CRS | Grade ≥ 2 CRS |
| 2/60 mg modified Dex | 11.3 (1.96, 11800) | 25.2 (13.7, 41.6) | 3.86 (1.16, 12.1) |
| 2/2/60 mg modified Dex | 8.90 (1.46, 3760) | 21.5 (11.6, 36.4) | 3.46 (1.06, 10.7) |
| 2/3/60 mg modified Dex | 8.64 (1.40, 3390) | 21.0 (11.3, 35.8) | 3.42 (1.05, 10.5) |
| 2/4/60 mg modified Dex | 8.46 (1.37, 3100) | 34.4 (24.7, 48.0) | 6.54 (4.20, 9.69) |

In addition, the CRS PK/PD analyses suggested that a 4 mg step-up dose/60 mg maintenance dose was likely to increase any-grade and >G2 CRS rates, compared to the 2 mg step-up dose/60 mg maintenance dose regimen.

In sum, the ER and CRS PK/PD analyses suggested that MPDR (36 mg) and 1-SUD improve the CRS safety profile of etentamig with no impact on efficacy (ORR) in patients with RRMM. Moreover, the findings suggested that implementation of 2-SUDs provides minimal additional improvement in CRS safety profile compared to 1-SUD. Hence, the evaluated 1-SUD dosing regimen of a 2 mg step-up dose and a modified premedication dose regimen prior to the maintenance dose of 60 mg Q4W is considered to be the optimal dosing regimen of etentamig.

1. A method for safely and effectively treating a human patient with multiple myeloma (MM) comprising administering to the patient during a first cycle comprising:
i) a first premedication dose of a corticosteroid, followed by a step-up dose of about 2 mg of etentamig, and
ii) a modified premedication dose regimen of a corticosteroid, followed by a maintenance dose of about 60 mg of etentamig,
wherein the first cycle has a duration of about four weeks, and
wherein said method reduces the incidence of one or more adverse events as compared to the administration of the maintenance dose without administration of a step-up dose.

2. (canceled)

3. The method of claim 1, wherein the first premedication dose of a corticosteroid comprises about 10 mg of dexamethasone.

4. The method of claim 3, wherein the about 10 mg of dexamethasone is orally administered to the patient about 15 to 60 minutes prior to administering the step-up dose of the etentamig.

5. The method of claim 1, wherein the corticosteroid is selected from the group consisting of: dexamethasone, hydrocortisone, prednisolone, and methylprednisolone.

6. The method of claim 5, wherein the first premedication dose of a corticosteroid comprises one of about 10 mg of dexamethasone, about 250 mg of hydrocortisone, about 62 mg of prednisolone, or about 50 mg of methylprednisolone.

7. The method of claim 6, wherein the about 10 mg of dexamethasone, about 250 mg of hydrocortisone, about 62 mg of prednisolone, or about 50 mg of methylprednisolone, is orally administered to the patient about 15 to 60 minutes prior to administering the step-up dose of the etentamig.

8. The method of claim 1, wherein the modified premedication dose regimen of a corticosteroid comprises about 36 mg of dexamethasone.

9. The method of claim 8, wherein the about 36 mg of dexamethasone comprises three separate administrations of dexamethasone.

10. The method of claim 9, wherein one administration of the dexamethasone comprises oral administration of about 8 mg of dexamethasone about 12-16 hours prior to administration of the maintenance dose of the etentamig.

11. The method of claim 8, wherein one administration of the dexamethasone comprises oral administration of about 8 mg of dexamethasone about 2-5 hours prior to administration of the maintenance dose of the etentamig.

12. The method of claim 8, wherein one administration of the dexamethasone comprises oral or intravenous administration of about 20 mg of dexamethasone about 15-60 minutes prior to administration of the maintenance dose of the etentamig.

13. The method of claim 1, wherein the modified premedication dose regimen of a corticosteroid comprises one of about 36 mg of dexamethasone, about 900 mg of hydrocortisone, about 225 mg of prednisolone, or about 180 mg of methylprednisolone.

14. The method of claim 13, wherein the about 36 mg of dexamethasone, the about 900 mg of hydrocortisone, the about 225 mg of prednisolone, or the about 180 mg of methylprednisolone, comprises three separate administrations.

15. The method of claim 14, wherein one administration of the dexamethasone,
hydrocortisone, prednisolone, or methylprednisolone comprises oral administration of one of about 8 mg of dexamethasone, about 200 mg of hydrocortisone, about 50 mg of prednisolone, or about 40 mg of methylprednisolone, about 12-16 hours prior to administration of the maintenance dose of the etentamig.

16. The method of claim 14, wherein one administration of the dexamethasone, hydrocortisone, prednisolone, or methylprednisolone comprises oral administration of one of about 8 mg of dexamethasone, about 200 mg of hydrocortisone, about 50 mg of prednisolone, or about 40 mg of methylprednisolone, about 2-5 hours prior to administration of the maintenance dose of the etentamig.

17. The method of claim 14, wherein one administration of the dexamethasone, hydrocortisone, prednisolone, or methylprednisolone comprises oral or intravenous administration of one of about 20 mg of dexamethasone, about 500 mg of hydrocortisone, about 125 mg of prednisolone, or about 100 mg of methylprednisolone, about 15-60 minutes prior to administration of the maintenance dose of the etentamig.

18.-20. (canceled)

21. The method of claim 1, wherein the method further comprising one or more subsequent cycles after the first cycle.

22.-35. (canceled)

36. A method of therapeutically effectively treating MM in a human patient in need thereof, wherein the method comprises:
(A) during a first cycle, administering to the patient about 62 mg of etentamig; and
(B) during one or more subsequent cycles, administering to the patient about 60 mg of the etentamig,
wherein each cycle has a duration of about four weeks, thereby treating said MM.

37. (canceled)

38. The method of claim 36, wherein the patient is an individual within a population of subjects receiving said treatment, and wherein the proportion of subjects within said population that achieves an objective response rate (ORR) is at least about 60%.

39. The method of claim 36, wherein the patient is an individual within a population of subjects receiving said treatment, and wherein the proportion of subjects within said population that achieves a very good partial response (VGPR) is at least about 50%.

40. The method of claim 36, wherein the patient is an individual within a population of subjects receiving said treatment, and wherein the proportion of subjects within said population that achieves a complete response (CR) is at least about 5%.

41. A method for the safe and effective treatment of MM in a patient in need thereof, wherein the method comprises:
(A) during a first cycle,
i) administering to the patient a first premedication dose of a corticosteroid,
ii) subsequently administering to the patient a step-up dose of about 2 mg of etentamig,
iii) subsequently administering to the patient one or more additional premedication doses of a corticosteroid,
iv) subsequently administering to the patient a maintenance dose of about 60 mg of the etentamig; and
(B) during one or more subsequent cycles, administering to the patient the maintenance dose of the etentamig;
wherein each of the first and subsequent cycles have a duration of approximately four weeks; and
wherein the method results in a reduced incidence of one or more adverse events as compared to a second method comprising administering etentamig without one or more doses of a corticosteroid.

42. The method of claim 41, wherein the corticosteroid is dexamethasone.

43. The method of claim 41, wherein the first premedication dose comprises 10 mg of dexamethasone administered orally.

44. The method of claim 41, wherein the one or more additional premedication doses comprises two oral doses of 8 mg dexamethasone, and one oral or intravenous dose of 20 mg dexamethasone.

45. The method of claim 44, wherein:
the first 8 mg dexamethasone dose is administered orally about 12-16 hours prior to the administration of the etentamig maintenance dose;
the second 8 mg dexamethasone dose is administered orally about 2-5 hours prior to administration of the etentamig maintenance dose; and
the 20 mg dexamethasone dose is administered orally or intravenously less than one hour prior to the administration of the etentamig maintenance dose.

46. The method of claim 41, wherein the patient is administered a total of about 20 to about 60 mg dexamethasone during the first cycle.

47. The method of claim 41, wherein the one or more adverse events are selected from the group consisting of cytokine release syndrome (CRS), neutropenia, anemia, thrombocytopenia, fatigue, diarrhea, decreased neutrophil count, nausea, and decreased lymphocyte count.

48. The method of claim 47, wherein the adverse event is CRS.

49.-66. (canceled)

67. A method for the safe and effective treatment of MM in a patient in need thereof, comprising administering to the patient
(A) during a first cycle:
i) on day 1 (but prior to step ii), an oral premedication dose of about 10 mg of dexamethasone,
ii) on day 1, a step-up dose of about 2 mg of etentamig,
iii) on day 3 (or up to day 13 but prior to step v), an oral premedication dose of about 8 mg of dexamethasone,
iv) on day 3 (or up to day 13 but prior to step v), an oral premedication dose of about 8 mg of dexamethasone,
v) on day 4 (or up to day 14), an oral or intravenous premedication dose of about 20 mg of dexamethasone,
vi) on day 4 (or up to day 14), a maintenance dose of about 60 mg of the etentamig; and
(B) during a subsequent cycle:
i) on day 1, the maintenance dose of the etentamig,
wherein each cycle comprises about 28 days, and
wherein the method results in a greater reduction in CRS without reduction of overall response rate in treating said MM as compared to administration of the maintenance dose of the etentamig without the step-up dose of the etentamig during the first cycle.
